(19) United States
(12) Patent Application Publication
(10) Pub. No.: US 2025/0270445 A1
(43) Pub. Date: Aug. 28, 2025

(54) COMPOUNDS AND USES THEREOF IN FIELD OF OPTOELECTRONICS

(71) Applicant: Zhejiang Brilliant Optoelectronic Technology Co.,Ltd., Taizhou (CN)

(72) Inventors: Junyou PAN, Taizhou (CN); Shuang HE, Taizhou (CN); Juye ZHU, Taizhou (CN); Markus MUEHLINGHAUS, Taizhou (CN); Klaus MUELLEN, Taizhou (CN)

(73) Assignee: Zhejiang Brilliant Optoelectronic Technology Co.,Ltd., Taizhou (CN)

(21) Appl. No.: 19/209,587

(22) Filed: May 15, 2025

Related U.S. Application Data
(63) Continuation of application No. PCT/CN2023/132406, filed on Nov. 17, 2023.

(30) Foreign Application Priority Data
Nov. 17, 2022 (CN) 202211441864.8

(51) Int. Cl. C09K 11/06; C07C 211/61; C07D 213/74; C07D 215/38; C07D 215/40; C07D 215/44; C07D 233/88; C07D 239/42; C07D 239/72; C07D 241/20; C07D 241/42; C07D 263/48; C07D 277/42; C07D 307/79; C07D 307/82; C07D 333/36; C07D 333/54; C07D 333/66; C07D 519/00; C08F 212/08; H10K 59/38

(52) U.S. Cl. CPC C09K 11/06; C07C 211/61; C07D 213/74; C07D 215/38; C07D 215/40; C07D 215/44; C07D 233/88; C07D 239/42; C07D 239/72; C07D 241/20; C07D 241/42; C07D 263/48; C07D 277/42; C07D 307/79; C07D 307/82; C07D 333/36; C07D 333/54; C07D 333/66; C07D 519/00; C08F 212/08; H10K 59/38; C07C 2603/50; C08F 2800/10; C09K 2211/1007; C09K 2211/1011; C09K 2211/1014; C09K 2211/1416; C09K 2211/1425; C09K 2211/1433

(57) ABSTRACT

Disclosed are compounds including a structural unit of formula (I). Also provided are organic functional films containing the compounds. Further provided are optoelectronic devices containing the compounds and the organic functional films. The compound according to the present disclosure has a large molar extinction coefficient and fluorescence emission efficiency, and a color conversion layer made of the compound can effectively absorb incident light, facilitating preparation of a thin color conversion layer; moreover, the absorption spectrum of the compound can be adjusted by modifying the molecular structure of the compound, different types of color conversion layers can be prepared by using compounds having different chemical structures to absorb light of different colors, and display devices having high color gamut can be manufactured by using the color conversion layers in combination with light-emitting materials having narrow full widths at half maximum and different colors.

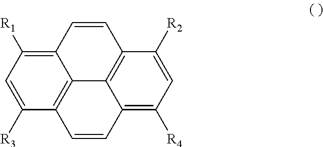

### CROSS-REFERENCE TO RELATED APPLICATIONS

The present application is a continuation of International Application No. PCT/CN2023/132406, filed on Nov. 17, 2023, which claims priority to Chinese Patent Application No. 202211441864.8, filed on Nov. 17, 2022. All of the aforementioned applications are incorporated herein by reference in their entireties.

### TECHNICAL FIELD

The present disclosure relates to the field of organic optoelectronic material and device technology, and in particular to a compound, an organic functional film, an optoelectronic device, and the applications thereof in the optoelectronic field.

### BACKGROUND

According to the principles of colorimetry, the narrower the full width at half maximum (FWHM) of the lights perceived by the human eyes is, the higher the color purity, and the more vivid the color display would be. Display devices with narrow-FWHM red, green and blue primary light are able to show vivid views with high color gamut and high visual quality.

The current mainstream full-color displays are achieved mainly in two ways. The first method is to actively emit red, green and blue lights, typically such as red-green-blue-organic light-emitting diode (RGB-OLED) display. The current mature technology is to fabricate light-emitting devices with three colors by vacuum evaporation with fine metal masks, which is complex, at high cost and difficult to achieve high-resolution display over 600 ppi. The second method is using color converters to convert the single-color light from the light-emitting devices into different colors, thereby achieving a full-color display. For example, Samsung combines blue organic light-emitting diodes (OLEDs) with red and green quantum dots (QD) films as the color converters. In this case, the fabrication of the light-emitting devices is much simpler, and thus higher yield. Furthermore, the manufacture of the color converters can be achieved by different technologies, such as vacuum evaporation, ink-jet printing, transfer printing and photolithography, etc., appliable to a variety of display products with very different resolution requirements from low resolution large-size television (TV) (around only 50 ppi) to high resolution silicon-based micro-display (over 3000 ppi).

Currently, there are mainly two types of color conversion materials used in mainstream color converters. The first one is an inorganic nanocrystal, commonly known as a quantum dot, which is a nanoparticle (especially is a quantum dot) of an inorganic semiconductor material (InP, CdSe, CdS, ZnSe, etc.) with a diameter of 2 nm to 8 nm. Limited by the current synthesis and separation technology of quantum dots, the FWHMs of cadmium (CD)-containing quantum dots typically range from 25 nm to 40 nm, which meet the display requirements of NTSC for color purity. Meanwhile, Cd-free quantum dots generally come with larger FWHMs of 35 nm to 75 nm. In addition, the extinction coefficient is generally low, requiring thicker films, the typical 10 μm or more is needed to achieve complete absorption of blue light, which is a great challenge for mass production processes, especially for Samsung's technology of combing blue OLED with red-green quantum dots. The second one is an organic dye, comprising various organic conjugated small molecules with chromophores. This organic dye generally has high extinction coefficient, but the intra-molecular thermal relaxation and the large vibration energy are always non-negligible, leading to the large FWHM (typically over 60 nm) of its emission spectrum. In WO Publication No. 2022213993, the present inventor proposes the host-dopant concept for the color conversion layer, and the organic materials having a high molar extinction coefficient are chosen as the host, which could absorb the light from the light emitting unit and transfer it to the narrow emissive dopant, thereby realizing a thin color conversion layer. However, the organic materials used as the hosts tend to have insufficient stability, including light stability and thermal stability still need to be greatly improved.

Therefore, it is necessary to further develop the new host materials with high extinction coefficient, high light stability and high thermal stability as color conversion materials, so that to realize high color gamut in display devices.

### SUMMARY

In one aspect, the present disclosure provides a compound comprising a structural unit of formula (I),

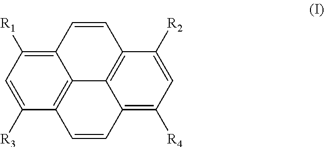

each of R₁ to R₄ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof;

Where at least three of R₁-R₄ are each independently selected from one of formulas (I-1)-(I-4):

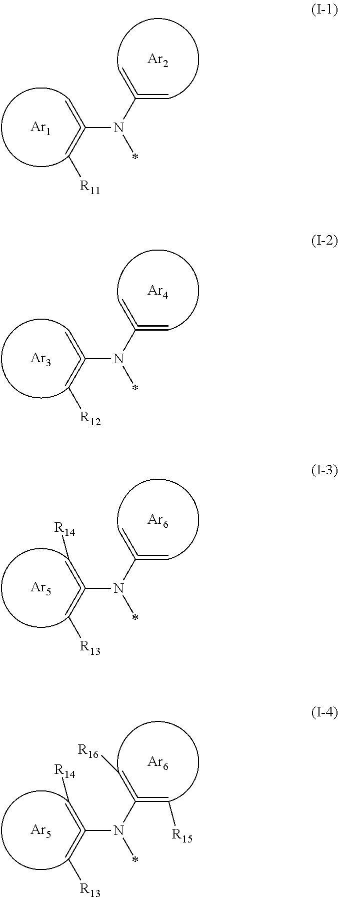

Where each * independently represents an attachment site connecting a pyrene; Ar₁ is a substituted/unsubstituted aromatic or heteroaromatic group containing 8 to 24 ring atoms; each of Ar₂ to Ar₆ is a substituted/unsubstituted aromatic or heteroaromatic group containing 5 to 24 ring atoms, and formula (I-2) comprises an electron-withdrawing group;

each of R₁₁ to R₁₆ is a substituent and independently selected from a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, or any combination thereof.

In another aspect, the present disclosure also provides a polymer comprising at least one repeating unit, where the at least one repeating unit comprises a structure corresponding to a compound as described herein.

In yet another aspect, the present disclosure further provides a mixture comprising at least one compound or polymer as described herein, and another functional material, the another functional material is an organic functional material, which may be selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye.

In yet another aspect, the present disclosure further provides a formulation comprising at least one compound or polymer or mixture as described herein, at least one organic solvent, and/or an organic resin.

In yet another aspect, the present disclosure further provides an organic functional film comprising a compound or a polymer or a mixture as described herein, or formed by using a formulation as described herein.

In yet another aspect, the present disclosure further provides an optoelectronic device comprising a compound or a polymer or a mixture or an organic functional film as described herein.

In addition or alternatively, the optoelectronic device is an organic light-emitting device comprising a substrate, a first electrode, an organic light-emitting layer, a second electrode, a color conversion layer, and an encapsulation layer in sequence from bottom to top, the second electrode is at least partially transparent, where 1) the color conversion layer comprises a compound or a polymer as described herein, and an emitter E; 2) the color conversion layer at least partially absorbs the light emitted by the organic light-emitting layer through the second electrode; 3) the emission spectrum of the compound or the polymer is on the short wavelength side of the absorption spectrum of the emitter E, and at least partially overlaps with the absorption spectrum of the emitter E; 4) the FWHM of the emission spectrum of the emitter E≤55 nm.

Beneficial effect: the compound as described herein has a large solubility, which enable the preparation of green inks for printing or coating processes; the absorption and emission spectrums thereof are less red-shifted or basically non-red-shifted after film formation; meanwhile, the compound has high stability, in particular has high light stability. Furthermore, the compound with a high extinction coefficient, and the resulting thin color converter, facilitate to achieve a high color gamut display.

### BRIEF DESCRIPTION OF THE DRAWINGS

FIG. 1 shows a schematic view of a red, green and blue (RGB) three-color display device.

FIG. 2 shows the absorption and emission spectrum of the compound 1 in the toluene.

FIG. 3 shows the absorption and emission spectrum of the compound 1 in the film.

FIG. 4 shows the absorption and emission spectrum of the compound 3 in the toluene.

FIG. 5 shows the absorption and emission spectrum of the compound 3 in the film.

FIG. 6 shows the absorption and emission spectrum of the compound 4 in the toluene.

FIG. 7 shows the absorption and emission spectrum of the compound 4 in the film.

FIG. 8 shows the absorption and emission spectrum of the compound 5 in the toluene.

FIG. 9 shows the absorption and emission spectrum of the compound 5 in the film.

FIG. 10 shows the absorption and emission spectrum of the compound 8 in the toluene.

FIG. 11 shows the absorption and emission spectrum of the compound 8 in the film.

FIG. 12 shows the absorption and emission spectrum of the compound 9 in the toluene.

FIG. 13 shows the absorption and emission spectrum of the compound 9 in the film.

FIG. 14 shows the absorption and emission spectrum of the compound 13 in the toluene.

FIG. 15 shows the absorption and emission spectrum of the compound 13 in the film.

FIG. 16 shows the absorption and emission spectrum of the compound 14 in the toluene.

FIG. 17 shows the absorption and emission spectrum of the compound 14 in the film.

FIG. 18 shows the absorption and emission spectrum of the compound 15 in the toluene.

FIG. 19 shows the absorption and emission spectrum of the compound 15 in the film.

FIG. 20 shows the absorption and emission spectrum of the compound 16 in the toluene.

FIG. 21 shows the absorption and emission spectrum of the compound 16 in the film.

FIG. 22 shows the absorption and emission spectrum of the compound 17 in the toluene.

FIG. 23 shows the absorption and emission spectrum of the compound 17 in the film.

FIG. 24 shows the absorption and emission spectrum of the compound 18 in the toluene.

FIG. 25 shows the absorption and emission spectrum of the compound 18 in the film.

FIG. 26 shows the absorption and emission spectrum of the compound 20 in the toluene.

FIG. 27 shows the absorption and emission spectrum of the compound 20 in the film.

FIG. 28 shows the absorption and emission spectrum of the compound E1 in the toluene.

FIG. 29 shows the absorption and emission spectrum of the compound E2 in the toluene.

FIG. 30 shows the absorption and emission spectrum of the compound E3 in the toluene.

FIG. 31 shows the absorption and emission spectrum of the comparative compound 1 in the toluene.

FIG. 32 shows the absorption and emission spectrum of the comparative compound 1 in the film.

FIG. 33 shows the absorption decay of the compound 1, compound 3, compound 4, compound 5, compound 8, compound 9, compound 13, compound 15, compound 16, compound 20, and comparative compound 2 after UV irradiation in the toluene.

FIG. 34 shows the luminance decay of the films of the compound 8 and the comparative compound 1 after blue-light illumination.

FIG. 35 shows the spectrum of the resin film of the CCL combined with the blue top-emitting OLED.

FIG. 36 shows the spectrum of the resin film of the CCL combined with the blue bottom-emitting OLED.

### DETAILED DESCRIPTION OF THE EMBODIMENTS

The present disclosure provides a compound, an organic functional film, an optoelectronic device, and the application thereof in organic light-emitting devices. In order to facilitate understanding of the present disclosure, the present disclosure will be described in detail below with reference to the accompanying drawings, in which the preferred embodiments of the present disclosure are shown. The present disclosure may be embodied in many different forms and is not limited to the embodiments described herein. Rather, these embodiments are provided so that the understanding of the disclosure of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “metal organic clathrate”, “metal organic complexe”, and “organomentallic complexe” have the same meaning, and they are interchangeable with each other.

As used herein, the terms “formulation”, “printing ink”, and “ink” have the same meaning, and they are interchangeable with each other.

In one aspect, the present disclosure provides a compound comprising a structural unit of formula (I),

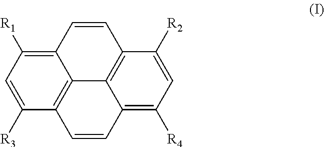

each of R₁ to R₄ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof;

Where at least three of R₁-R₄ are each independently selected from one of formulas (I-1)-(I-4):

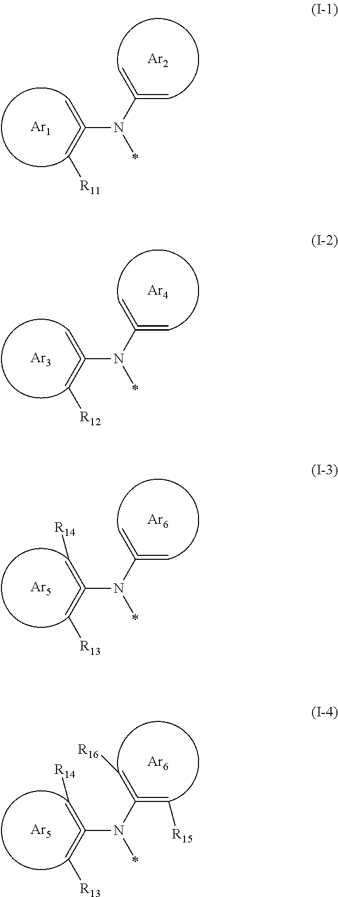

Where each * independently represents an attachment site connecting a pyrene; Ar₁ is a substituted/unsubstituted aromatic or heteroaromatic group containing 8 to 24 ring atoms; each of Ar₂ to Ar₆ is a substituted/unsubstituted aromatic or heteroaromatic group containing 5 to 24 ring atoms, and formula (I-2) comprises an electron-withdrawing group;

each of R₁₁ to R₁₆ is a substituent and independently selected from a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, or any combination thereof.

In some embodiments, R₁-R₄ are each independently selected from one of formulas (I-1)-(I-4).

In some embodiments, three of R₁-R₄ are the same structural unit.

In some embodiments, in R₁-R₄ as described herein, R₁ and R₃, or R₂ and R₄ are the same structural unit.

In some embodiments, in R₁-R₄ as described herein, R₁ and R₄, or R₂ and R₃ are the same structural unit.

In some embodiments, R₁-R₄ are the same structural unit.

In some embodiments, each of R₁₁ to R₁₆ at each occurrence is independently selected from a C₁-C₁₀ linear alkyl group, a C₁-C₁₀ linear haloalkyl group, a C₁-C₁₀ linear alkoxy group, a C₁-C₁₀ linear thioalkoxy group, a C₃-C₁₀ branched/cyclic alkyl group, a C₃-C₁₀ branched/cyclic haloalkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ ketone group, a C₂-C₁₀ alkoxycarbonyl group, a C₆-C₁₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 20 ring atoms, an aryloxy or heteroaryloxy group containing 5 to 20 ring atoms, an arylamine or heteroarylamine group containing 5 to 20 ring atoms, or any combination thereof.

For the purposes of the present disclosure, the aromatic ring systems contain 6 to 20 carbon atoms, the heteroaromatic ring systems contain 1 to 20 carbon atoms and at least one heteroatom, provided that the total number of the carbon atoms and the heteroatoms is at least 5. The heteroatoms are preferably selected from Si, N, P, O, S and/or Ge, particularly preferably selected from Si, N, P, O and/or S. For the purposes of the present disclosure, the aromatic or heteroaromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this disclosure.

For the purposes of the present disclosure, the H atoms on the compound may be substituted with R₂₀. R₂₀ is defined as the above-mentioned R₁₁, which may be preferably selected from: (1) a C₁-C₁₀ alkyl group, particularly preferably selected from the following groups: a methyl group, an ethyl group, a n-propyl group, an isopropyl group, a cyclopropyl group, a n-butyl group, an isobutyl group, a sec-butyl group, a tert-butyl group, a cyclobutyl group, a 2-methylbutyl group, a n-pentyl group, a n-hexyl group, a cyclohexyl group, a n-heptyl group, a cycloheptyl group, a n-octyl group, a cyclooctyl group, a 2-methylheptyl group, a trifluoromethyl group, a pentafluoroethyl group, a 2,2,2-trifluoroethyl group, a vinyl group, a 1-propenyl group, a butenyl group, a pentenyl group, a cyclopentenyl group, a hexenyl group, a cyclohexenyl group, a heptenyl group, a cycloheptenyl group, an octenyl group, a cyclooctenyl group, an ethynyl group, a propynyl group, a butynyl group, a pentynyl group, a hexynyl group, or an octynyl group; (2) a C₁-C₁₀ alkoxy group, particularly preferably methoxy, ethoxy, n-propoxy, isopropoxy, n-butoxy, isobutoxy, sec-butoxy, tert-butoxy, or 2-methylbutoxy; (3) a C₂-C₁₀ aryl or heteroaryl group, which may be monovalent or divalent depending on the application, and in each case can also be optionally substituted with R₂₀ and may be attached to an aromatic or heteroaromatic ring at any desired position, particularly preferably selected from the following: benzene, naphthalene, anthracene, pyrene, dihydropyrene, chrysene, fluoranthene, naphthacene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenimidazole, pyridimidazole, pyrazine-imidazole, quinoxaline-imidazole, oxazole, benzoxazole, naphthoxazole, anthracenazole, phenoxazole, isoxazole, 1,2-thiazole, 1,3-thiazole, benzothiazole, pyridazine, benzopyridazine, pyrimidine, benzopyrimidine, quinoxaline, pyrazine, phenazine, 1,5-naphthyridine, nitrocarbazole, benzocarboline, 1,10-phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, or benzothiadiazole. For the purposes of the present disclosure, aromatic and heteroaromatic ring systems are particularly considered to be, in addition to the above-mentioned aryl and heteroaryl groups, also refer to biphenylene, terphenylene, fluorene, spirobifluorene, dihydrophenanthrene, tetrahydropyrene, cis-indenofluorene, or trans-indenofluorene.

In some embodiments, Ar₁ in formula (I-1) is selected from one of the following structural formulas or any combination thereof, which can be further substituted:

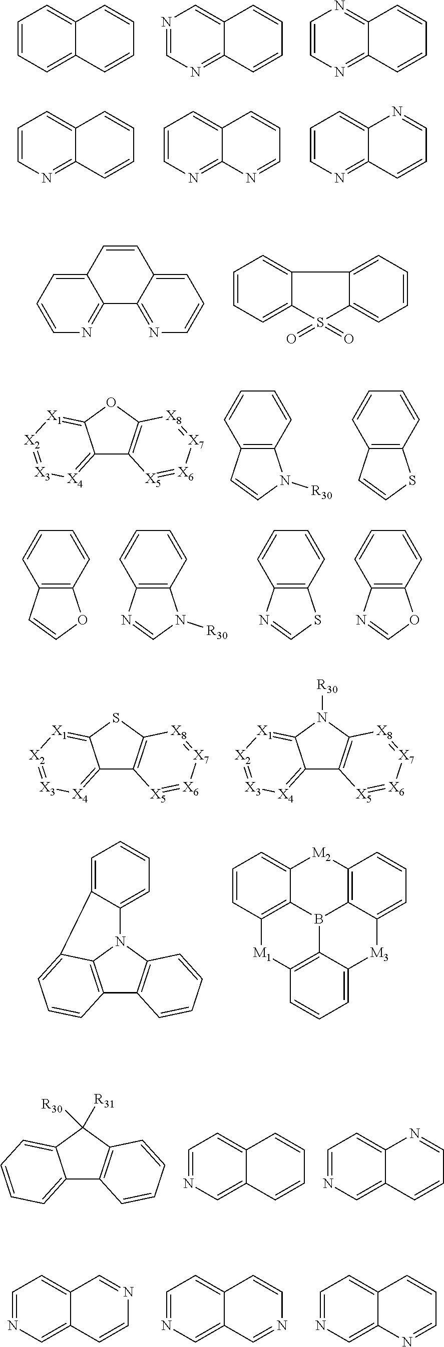

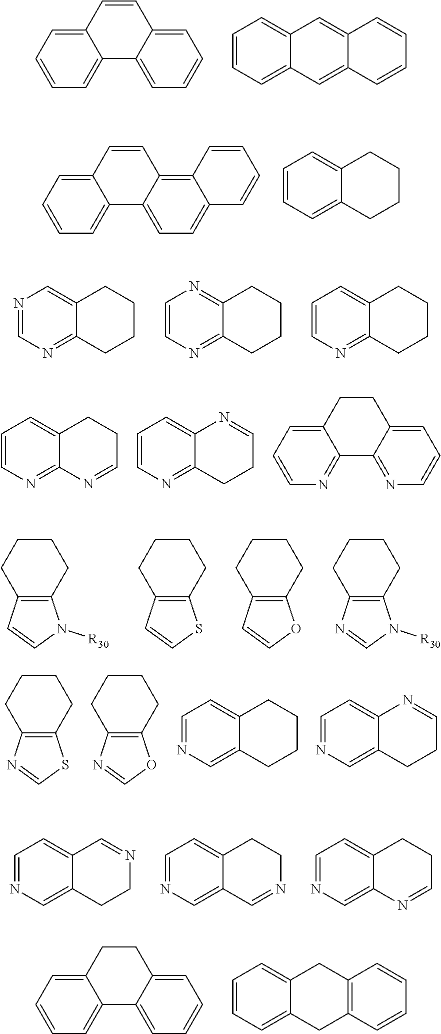

Where each of X₁ to X₈ is independently CR₃₂ or N; M₁, M₂, and M₃ are each independently selected from N(R₃₂), C(R₃₂R₃₃)₂, Si(R₃₂R₃₃)₂, O, C═N(R₃₂), C═C(R₃₂R₃₃)₂, P(R₃₂), P(═O)R₃₂, S, S═O, SO₂, or null; each of R₃₀ to R₃₃ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof, where one or more R₃₀-R₃₃ may form a monocyclic or polycyclic aliphatic or aromatic ring system with each other and/or with the rings bonded thereto.

In some embodiments, each Ar₁ is a naphthyl group.

In some embodiments, in the compound as described herein, Ar₂-Ar₆ are the same or different at each occurrence and are each independently selected from an aromatic or heteroaromatic group containing 5 to 20 ring atoms; preferably from an aromatic or heteroaromatic group containing 5 to 18 ring atoms; more preferably from an aromatic or heteroaromatic group containing 5 to 15 ring atoms; and most preferably from an aromatic or heteroaromatic group containing 5 to 10 ring atoms; they may be unsubstituted or substituted with one or two R₂₀. Preferred aromatic or heteraromatic groups include benzene, naphthalene, anthracene, phenanthrene, pyridine, benzofuran, pyrene, or thiophene.

In some embodiments, each of Ar₂ to Ar₆ at each occurrence is independently selected from the following structural formulas:

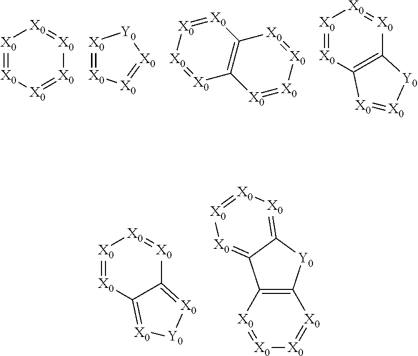

Where each X₀ is independently CR₄₀ or N; each Y₀ is independently selected from CR₄₁R₄₂, SiR₄₁R₄₂, NR₄₁, C(═O), S, or O; R₄₀-R₄₂ are identically defined as the above-mentioned R₃₀.

Further, each of Ar₂ to Ar₆ at each occurrence is independently selected from one of the following structural formulas or any combination thereof, which can be further arbitrarily substituted:

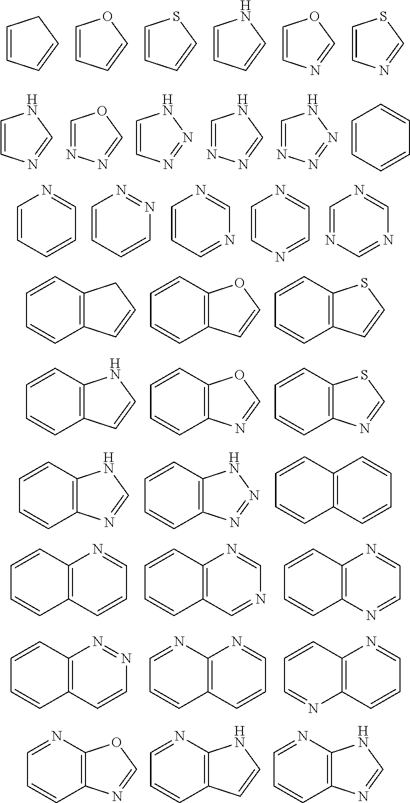

In some embodiments, each of Ar₂ to Ar₆ is a phenyl group.

Preferably, Ar₃ or Ar₄ in formula (I-2) is an electron-withdrawing group or substituted with an electron-withdrawing group.

In some embodiments, each of Ar₃ and Ar₄ is an electron-withdrawing group or substituted with an electron-withdrawing group.

In some embodiments, Ar₄ is an electron-withdrawing group or substituted with an electron-withdrawing group.

In some embodiments, formula (I-2) comprises two electron-withdrawing groups.

In some embodiments, formula (I-2) comprises three electron-withdrawing groups.

In some embodiments, formula (I-2) comprises more than three electron-withdrawing groups.

The above-mentioned electron-withdrawing group may be selected from F, a cyano group, a partial/perfluorinated alkyl chain, or one of the following groups:

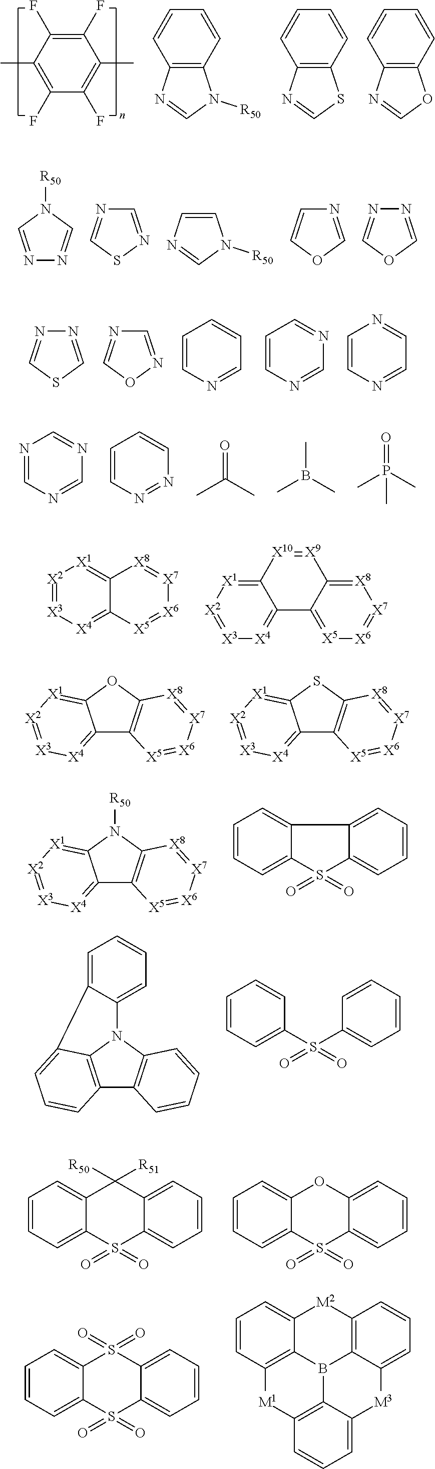

Where n is an integer from 1 to 3; each of X¹ to X¹⁰ is independently CR₅₂ or N, and at least one of them is N; M¹, M², and M³ are each independently selected from N(R₅₃), C(R₅₃R₅₄)₂, Si(R₅₃R₅₄)₂, O, C═N(R₅₃), C═C(R₅₃R₅₄)₂, P(R₅₃), P(═O)R₅₃, S, S═O, SO₂, or null; R₅₀-R₅₄ are identically defined as the above-mentioned R₃₀.

In some embodiments, the compound comprises F.

In some embodiments, the compound comprises CN.

In some embodiments, the compound comprises one of the following groups:

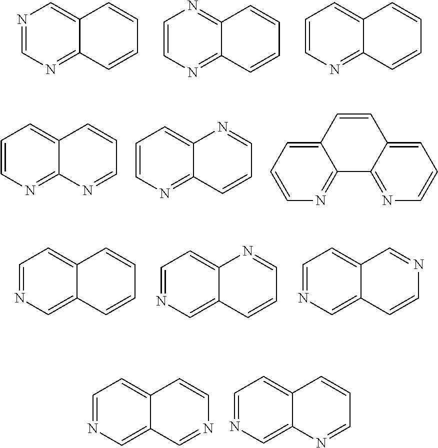

In some embodiments, R₁₃ and R₁₄ in formula (I-3) are different groups.

In some embodiments, R₁₃ and R₁₄ in formula (I-3) are the same group.

In some embodiments, each of R₁₃ and R₁₄ in formula (I-3) is independently selected from one of a methyl group, an ethyl group, or an isopropyl group.

In some embodiments, R₁₃-R₁₆ in formula (I-4) are the same or different groups.

In some embodiments, R₁₃ and R₁₄, or R₁₅ and R₁₆ in formula (I-4) are the same group.

In some embodiments, R₁₃-R₁₆ in formula (I-4) are the same group.

In some embodiments, each of R₁₃ to R₁₆ in formula (I-4) is a methyl group.

In some embodiments, in the compound as described herein, the total percentage of the SP³ hybrid groups does not exceed 50% of the total molecular weight, more preferably does not exceed 30%, and most preferably does not exceed 20%. The presence of less SP³ hybrid groups can effectively ensure the thermal stability of the compound, thereby ensuring the stability of the devices.

In some embodiments, in order to improve solubility and/or film-forming property, in the compound as described herein, the total percentage of the SP³ hybrid groups exceeds 20% of the total molecular, preferably exceeds 30%, more preferably exceeds 40%, and most preferably exceeds 50%.

In some embodiments, the compound has relatively high extinction coefficient. The extinction coefficient is also known as the molar extinction coefficient, which refers to the absorption coefficient at a concentration of 1 mol/L, and is represented by the symbol ε, in unit of Lmol⁻¹cm⁻¹. The extinction coefficient (ε) preferably ≥1*10³; more preferably ≥1*10⁴; even more preferably ≥2*10⁴; further preferably ≥3*10⁴; particularly preferably ≥5*10⁴; and most preferably ≥1*10⁵. Preferably, the extinction coefficient refers to the extinction coefficient at the wavelength corresponding to the absorption peak.

In some embodiments, the compound has a high fluorescence luminescence efficiency, and the photoluminescence quantum yield (PLQY) thereof ≥60%, preferably ≥65%, more preferably ≥70%, further preferably ≥80%, and most preferably ≥90%.

In embodiments of the present disclosure, the energy level structure of the organic materials, highest occupied molecular orbital (HOMO), lowest unoccupied molecular orbital (LUMO), and oscillator strength f play key roles. The determination of these parameters is introduced as follows.

HOMO and LUMO energy levels can be measured by optoelectronic effect, for example, by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The oscillator strength f can also be calculated by quantum simulation (such as Time-dependent DFT).

It should be noted that the absolute values of HOMO and LUMO may depend on the measurement method or calculation method used. Even for the same method, different ways of evaluation, for example, using either the onset or peak value of a CV curve as reference, may result in different HOMO/LUMO values. Therefore, reasonable and meaningful comparison should be carried out by employing the same measurement and evaluation methods. In the embodiments of the present disclosure, the values of HOMO and LUMO are based on the time-dependent DFT simulation, which however should not exclude the applications of other measurement or calculation methods.

The energy structure of the compounds has an important influence on its optoelectronic properties and stability.

In some embodiments, the compound has relatively low HOMO, generally ≤−4.6 eV, preferably ≤−4.7 eV, more preferably ≤−4.8 eV, even more preferably ≤−4.9 eV, further preferably ≤−5.1 eV, and most preferably ≤−5.2 eV.

In some embodiments, the compound has a large ΔHOMO and/or ΔLUMO, generally ≥0.30 eV, preferably ≥0.40 eV, more preferably ≥0.50 eV, further preferably ≥0.60 eV, and most preferably ≥0.70 eV; where ΔHOMO=HOMO−(HOMO−1), ΔLUMO=(LUMO+1)−LUMO.

For the purposes of the present disclosure, (HOMO−1) is defined as the energy level of the second highest occupied molecular orbital, (HOMO−2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on; these energy levels can be determined by the following simulation method.

In some embodiments, the compound has relatively large oscillator strength f(Sn) (n≥1); f(S1) generally ≥0.10, preferably ≥0.20, more preferably ≥0.30, even more preferably ≥0.40, further preferably ≥0.50, and most preferably ≥0.60. The oscillator strength f(Sn) may be calculated by the following method.

In some embodiments, f(S1)≥0.70, preferably ≥0.80, more preferably ≥0.90, even more preferably ≥1.00, further preferably ≥1.2, and most preferably ≥1.6.

In some embodiments, the compound has a high solubility in organic solvents. Preferably, the compound as described herein has a solubility of ≥10 mg/mL in toluene, preferably ≥20 mg/mL, more preferably ≥40 mg/mL, even more preferably ≥70 mg/mL, further preferably ≥100 mg/mL, and most preferably ≥150 mg/mL.

Examples of some suitable compounds are listed below (but not limited thereto), which can be further arbitrarily substituted:

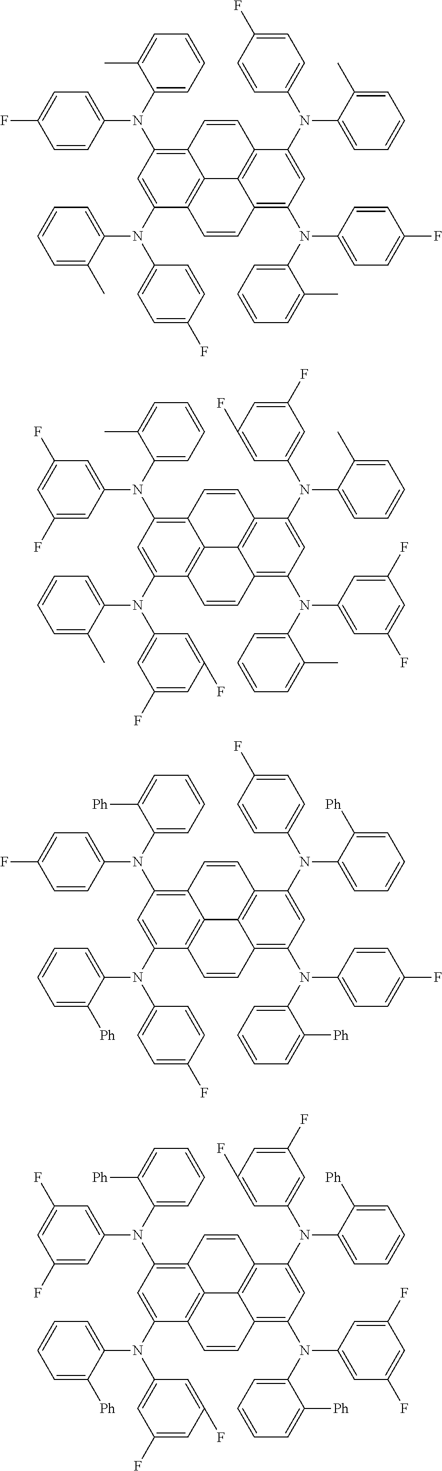

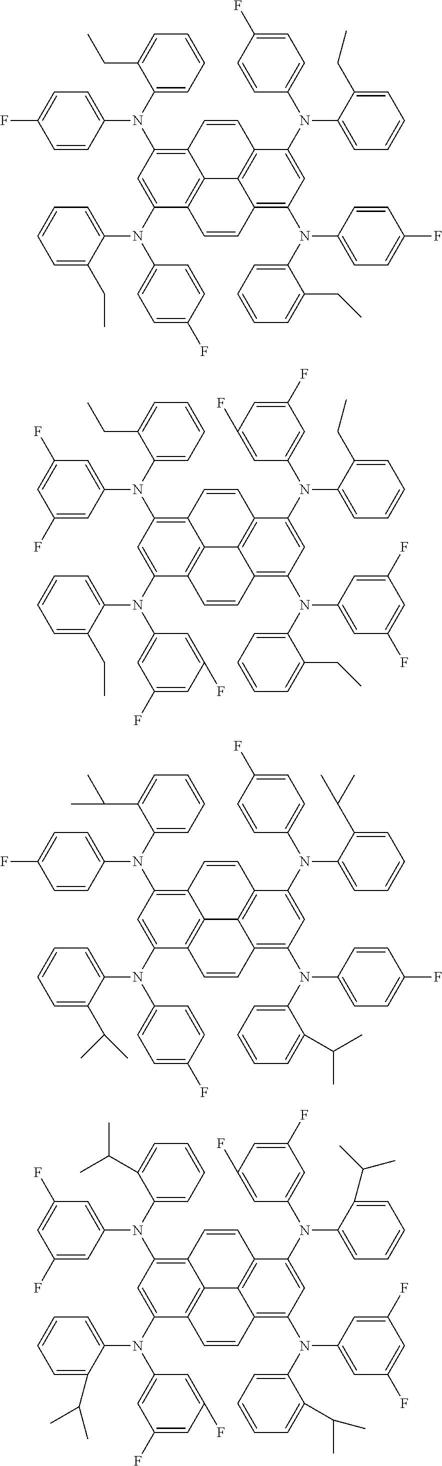

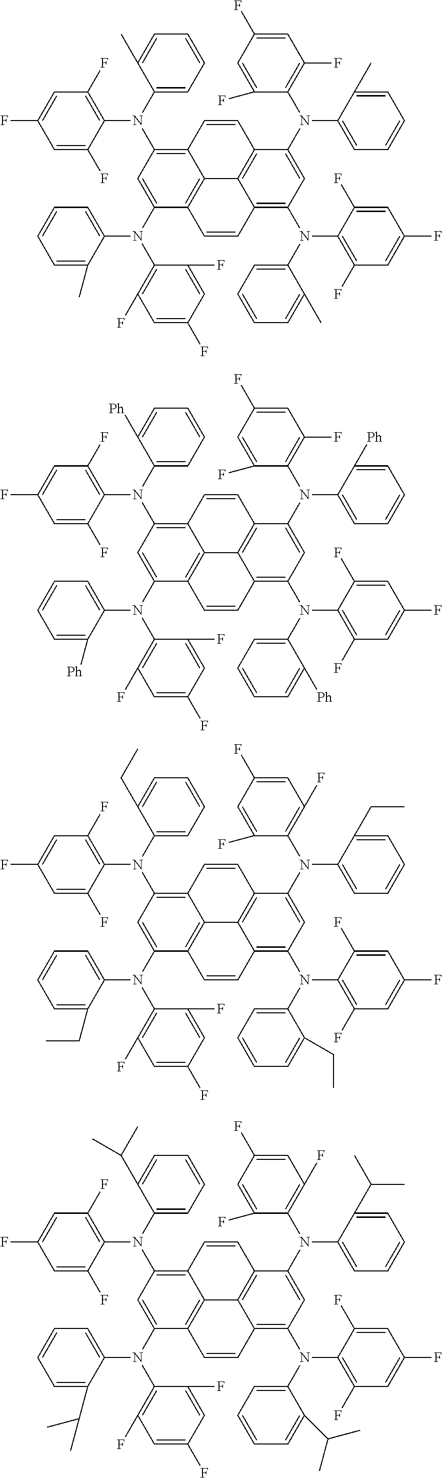

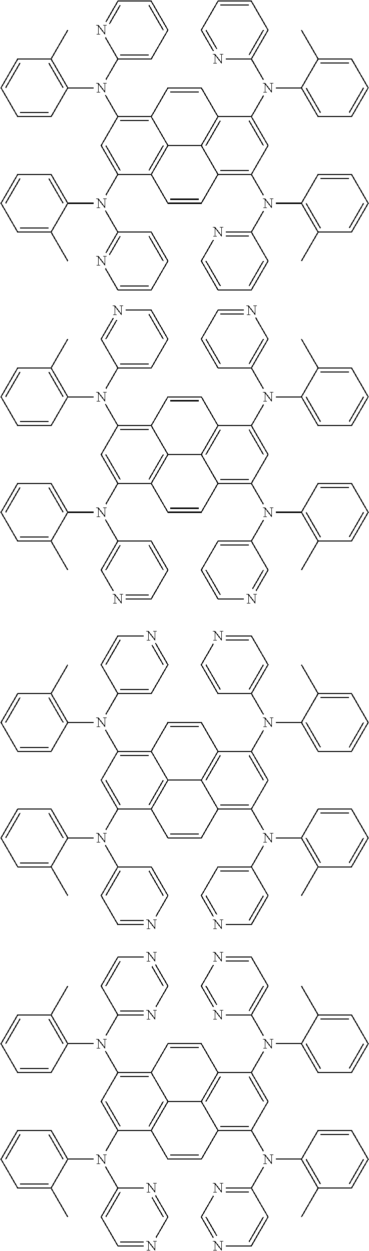

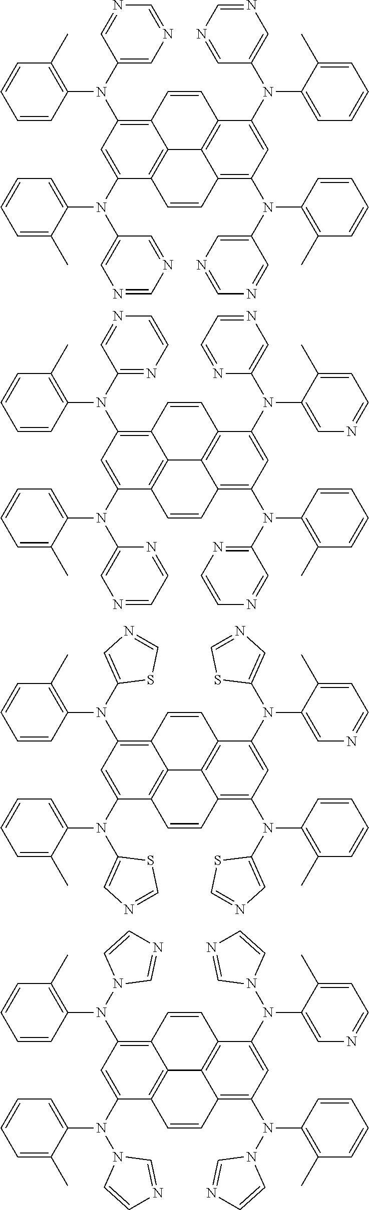

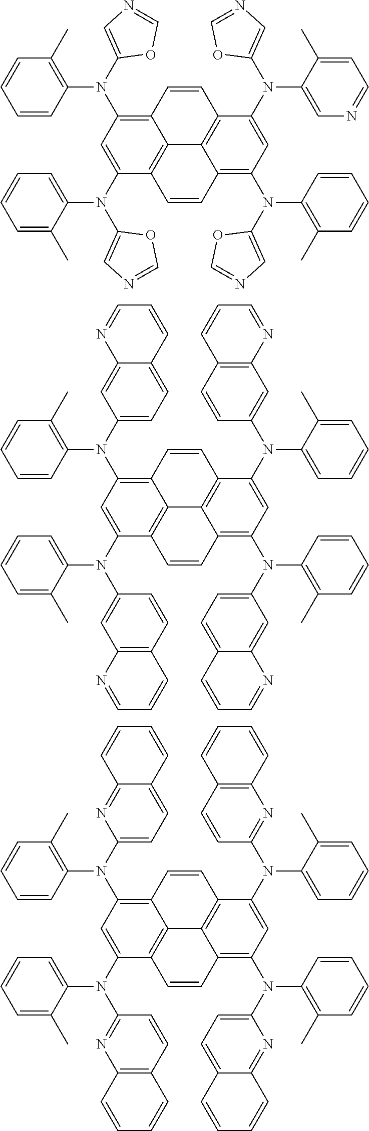

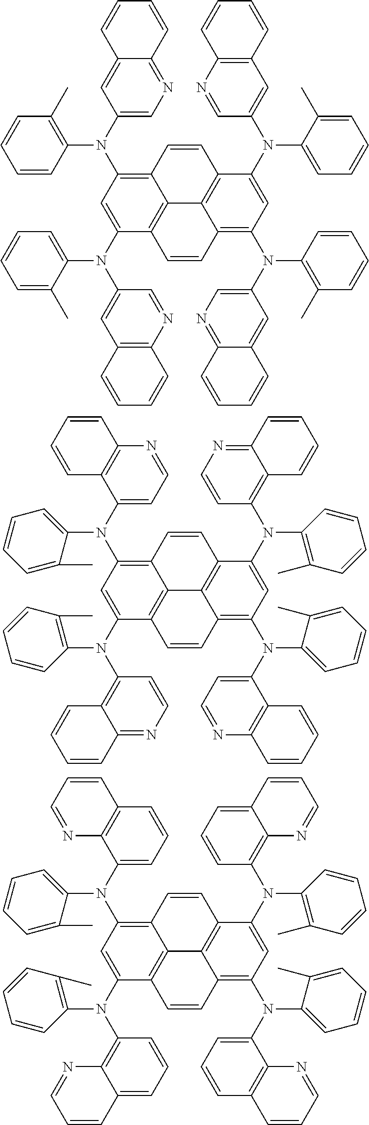

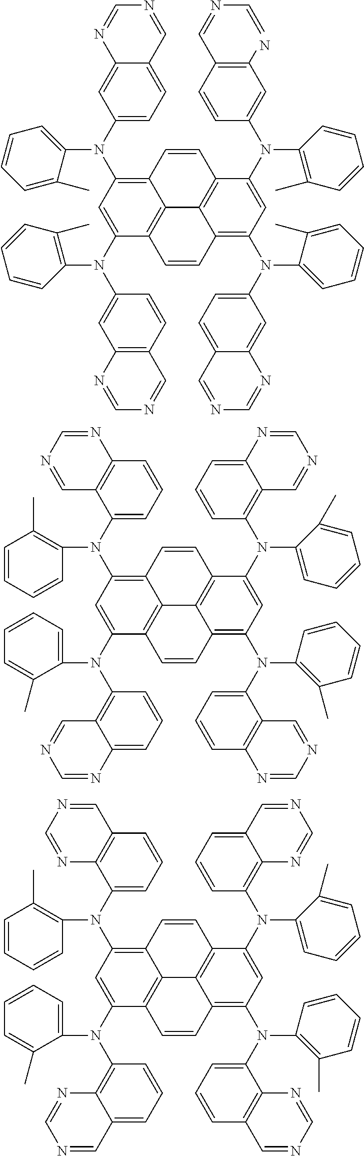

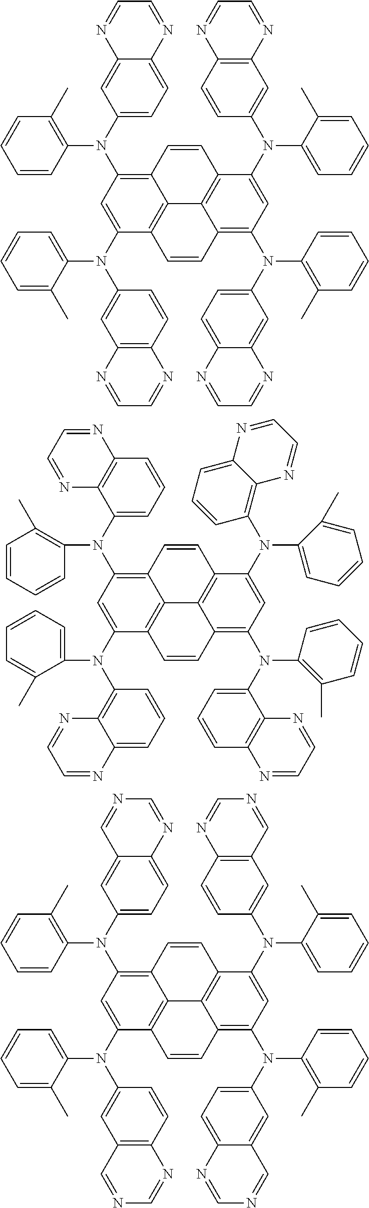

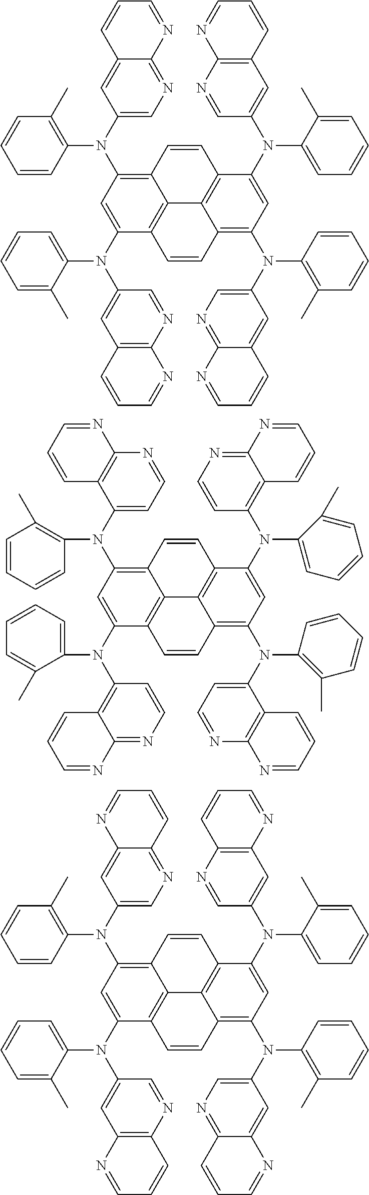

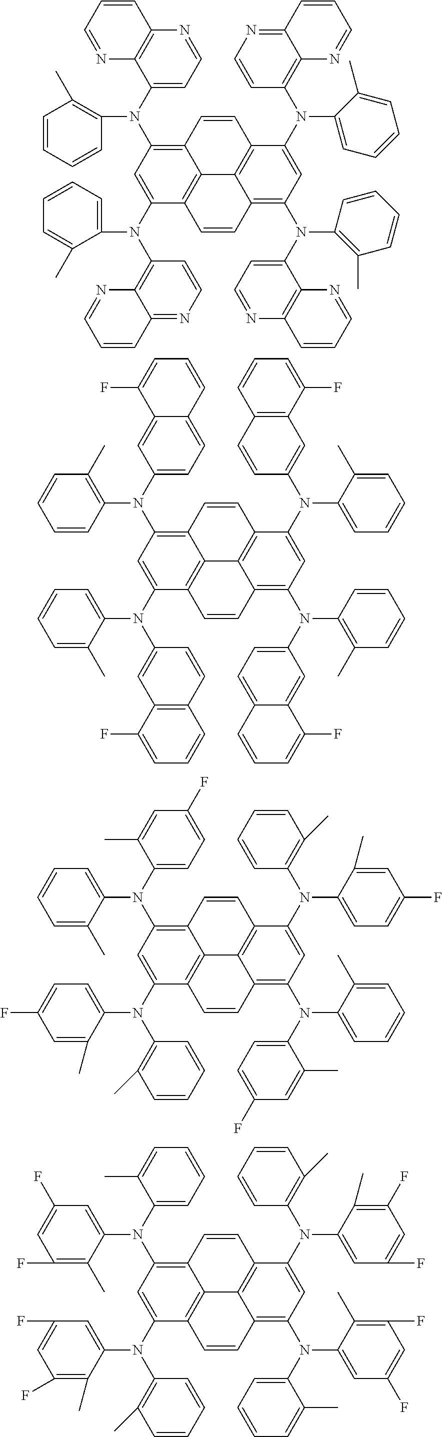

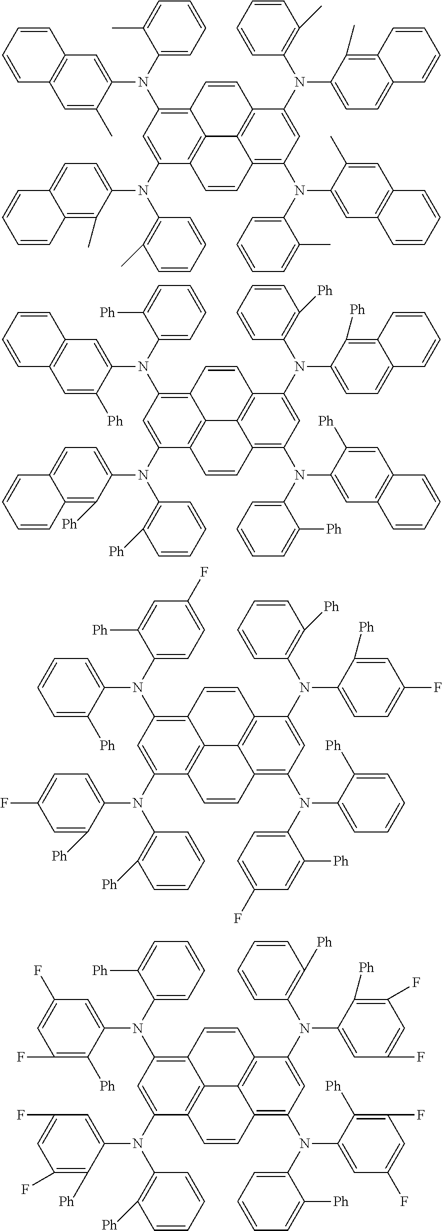

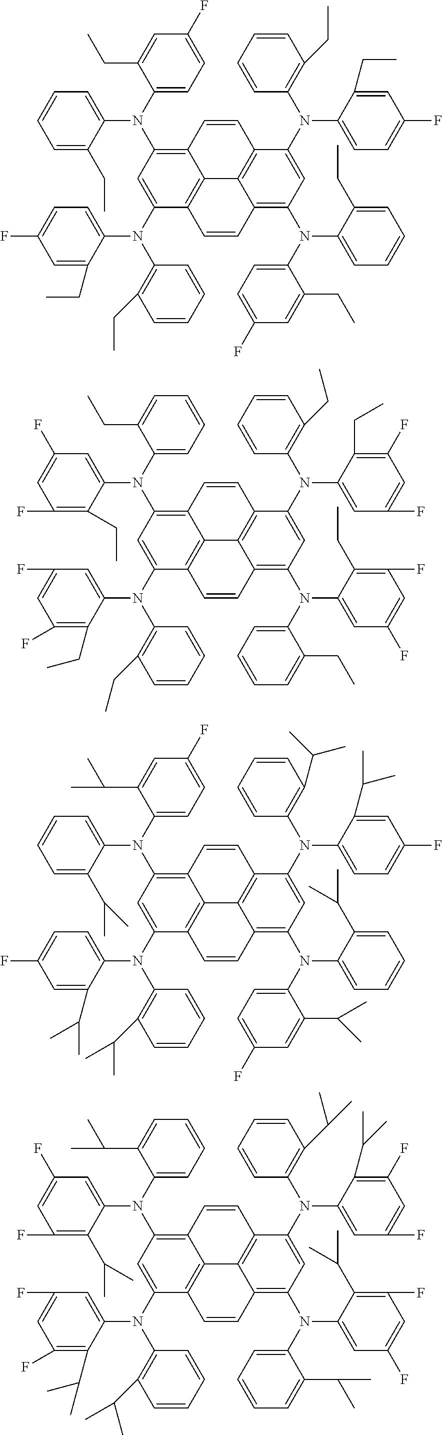

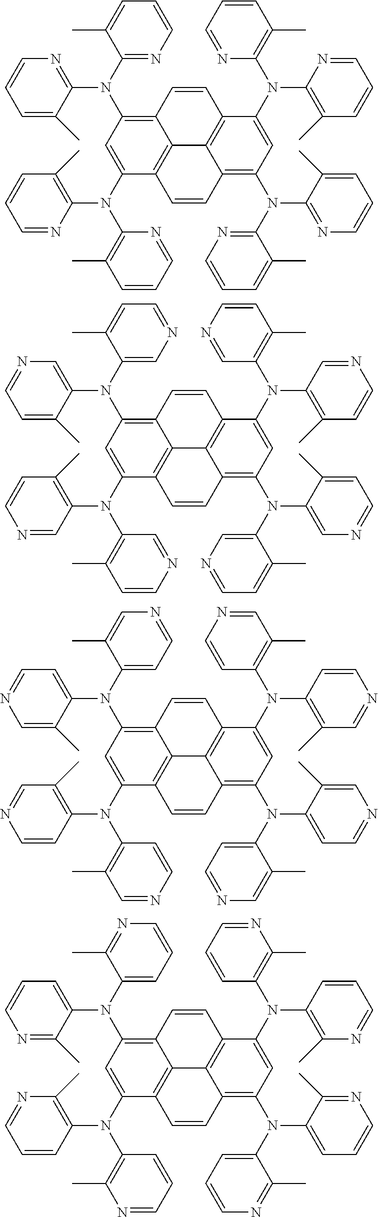

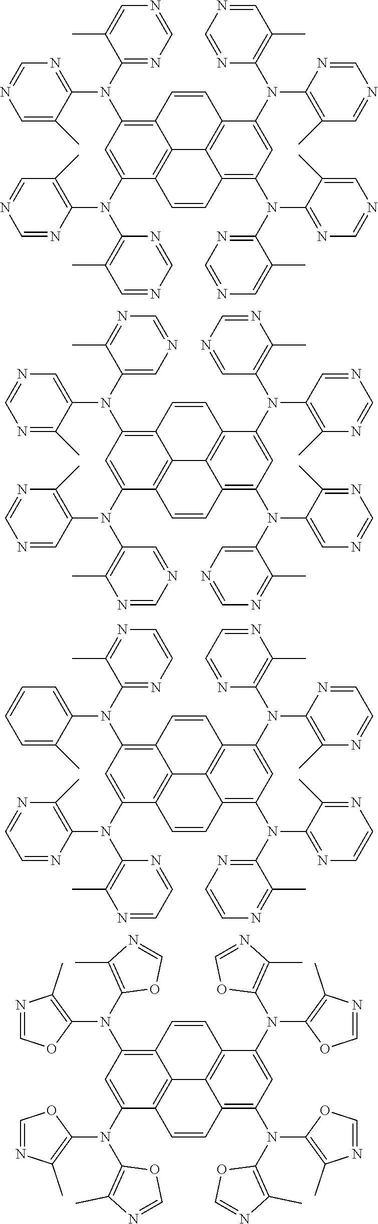

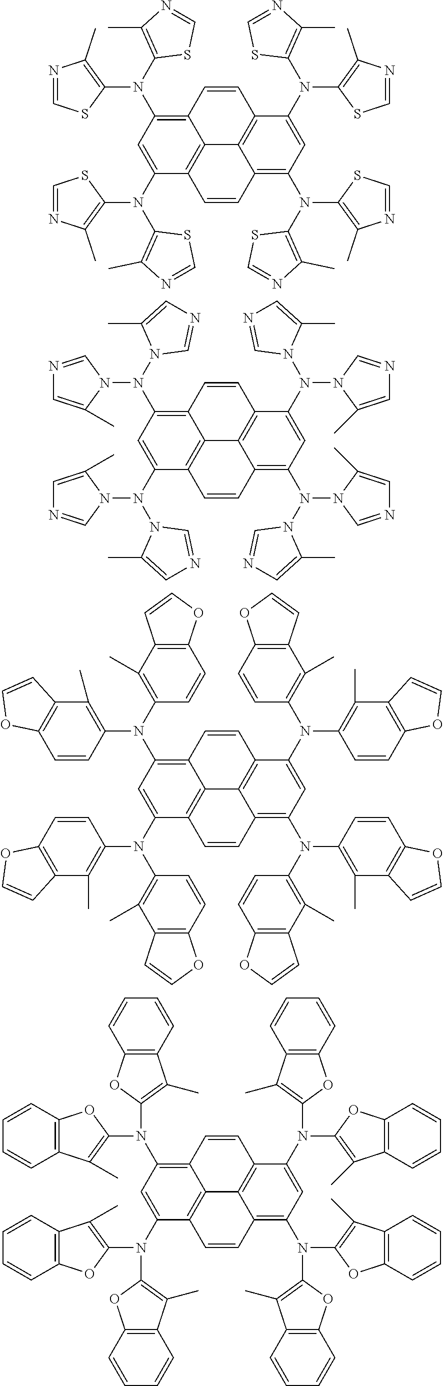

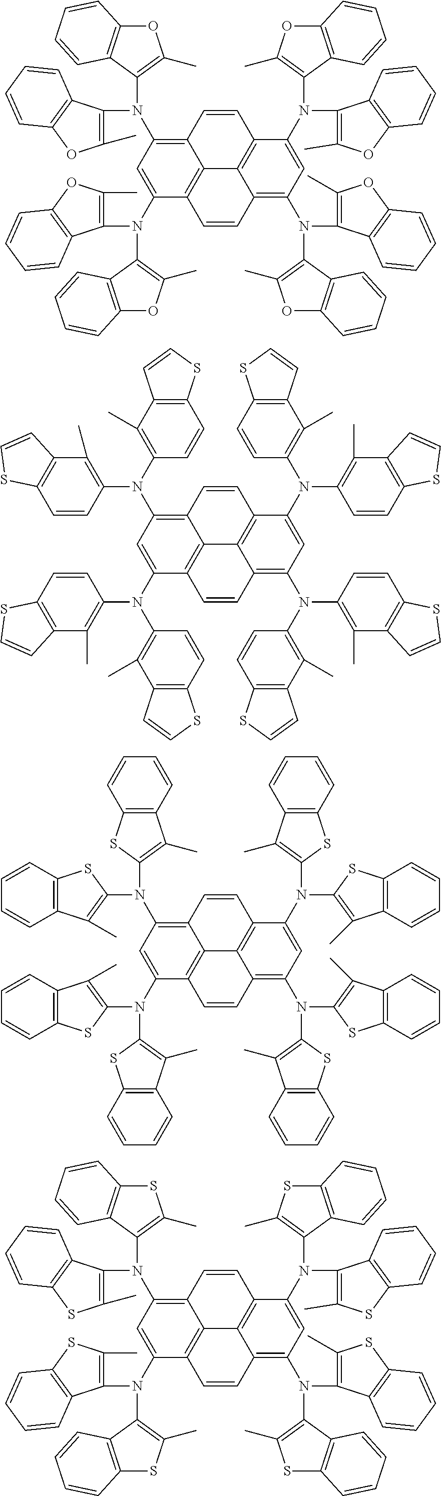

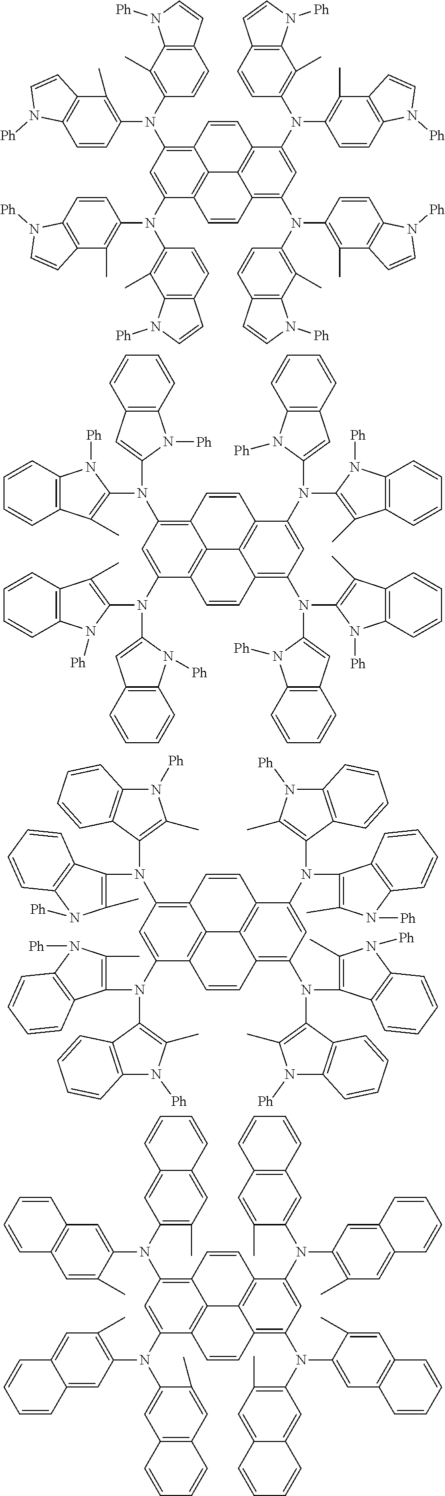

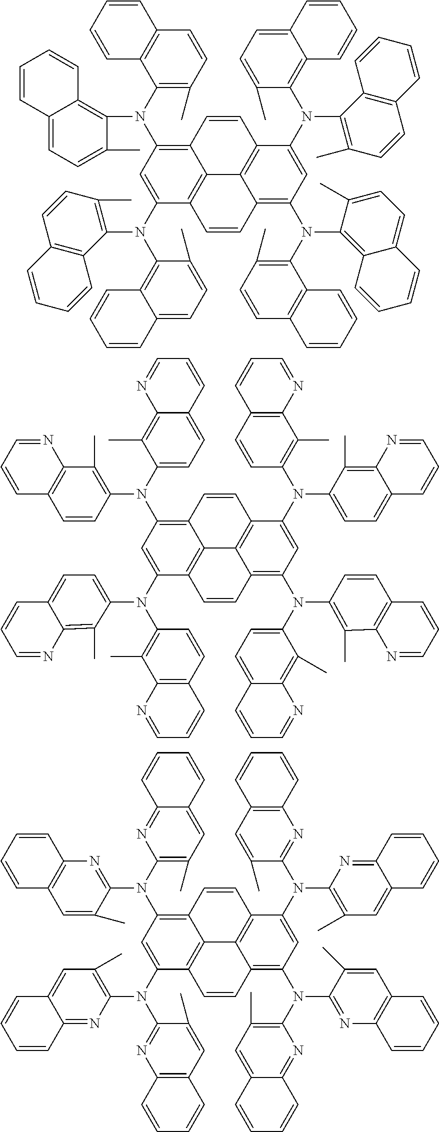

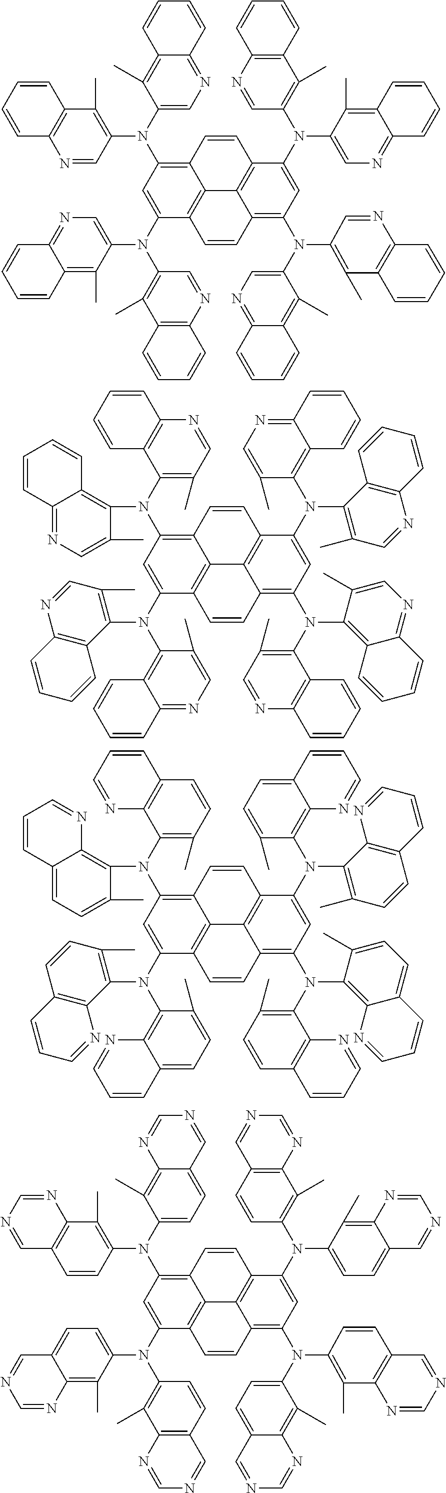

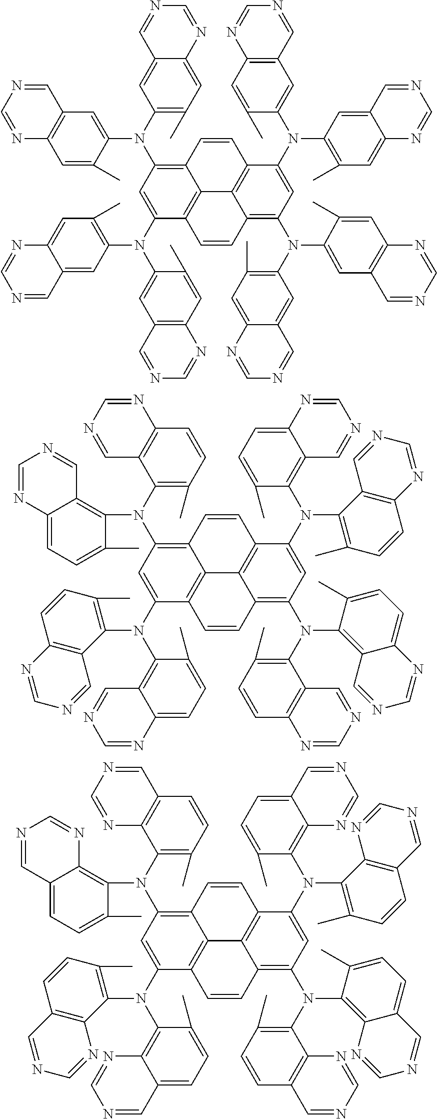

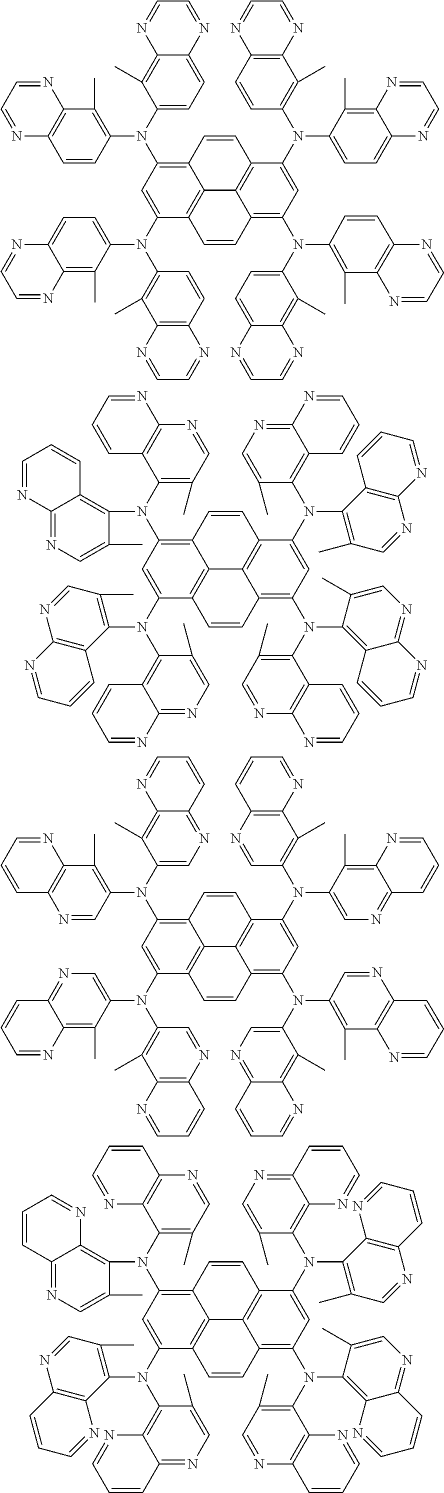

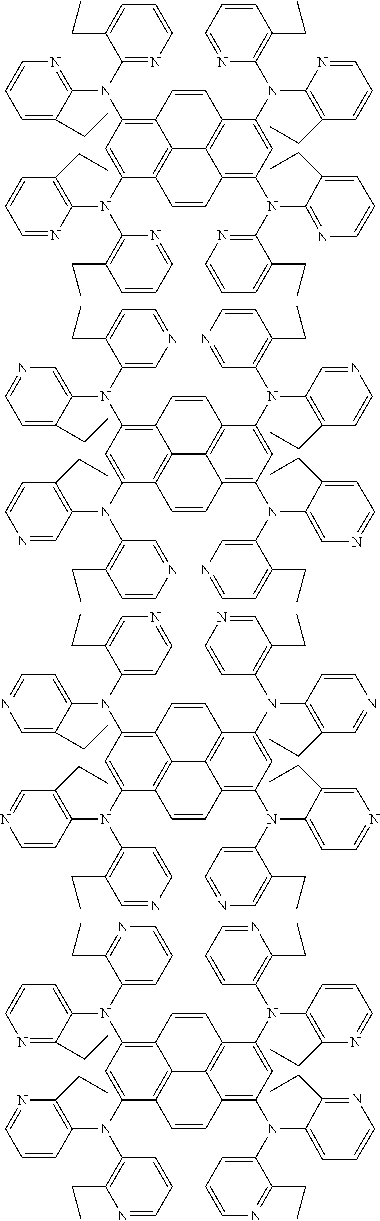

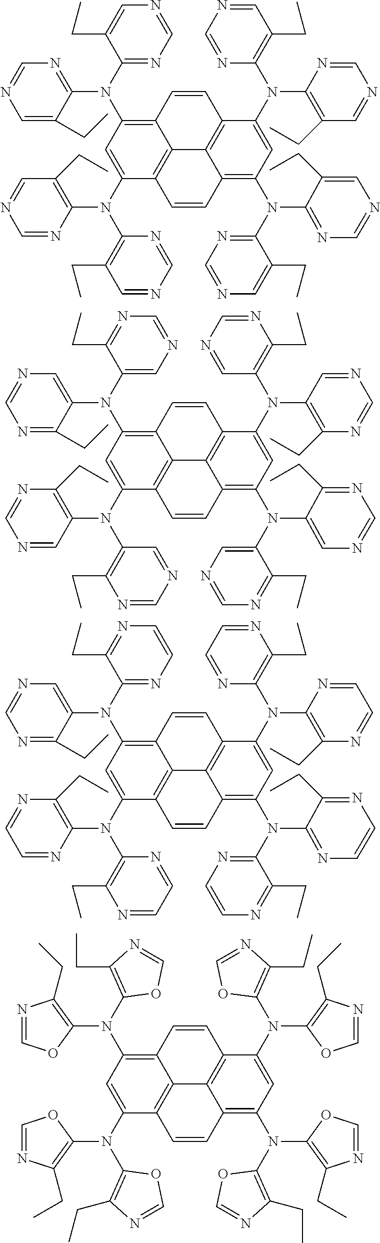

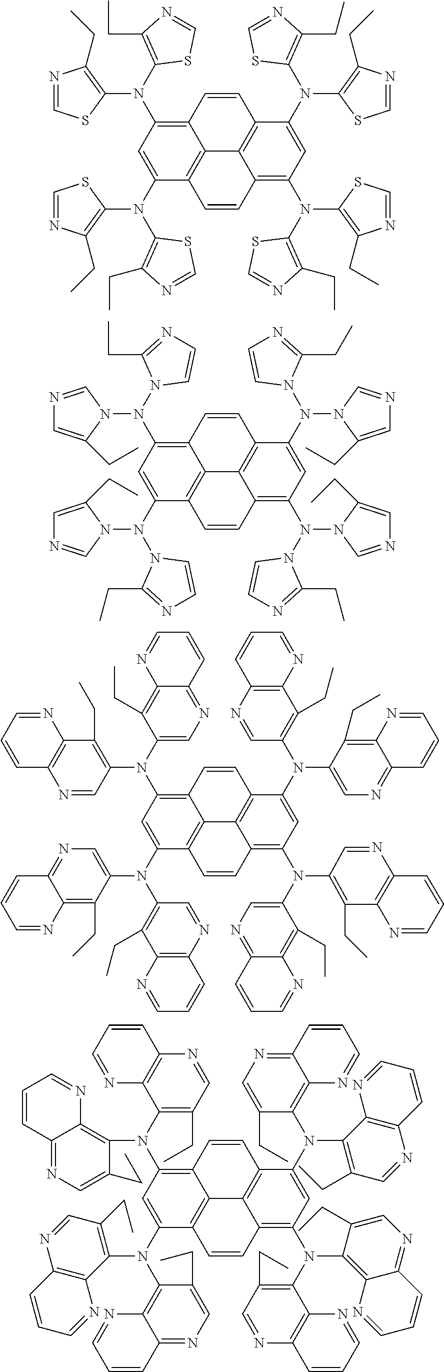

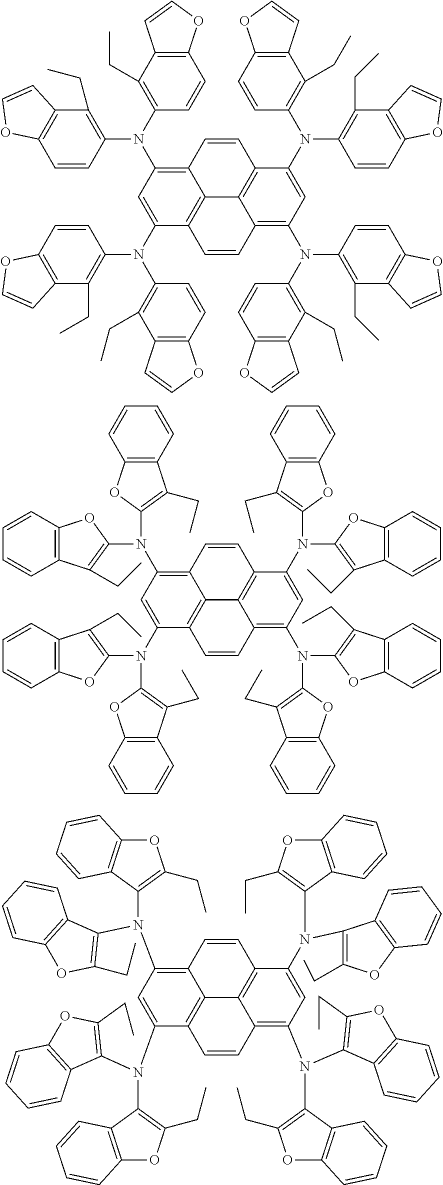

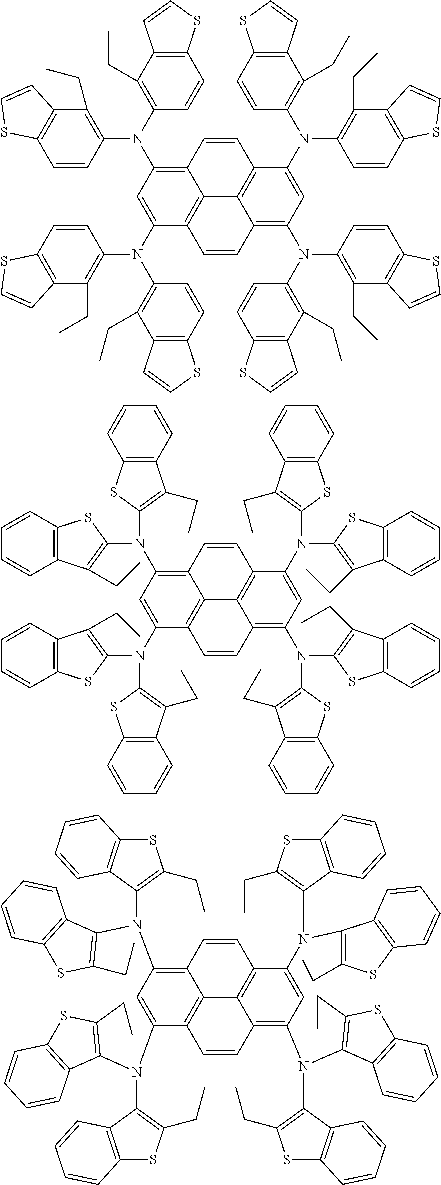

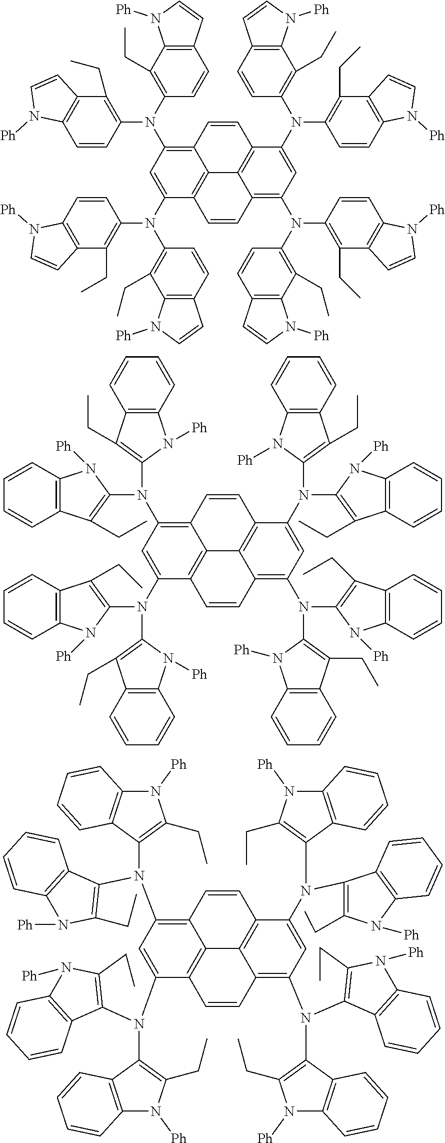

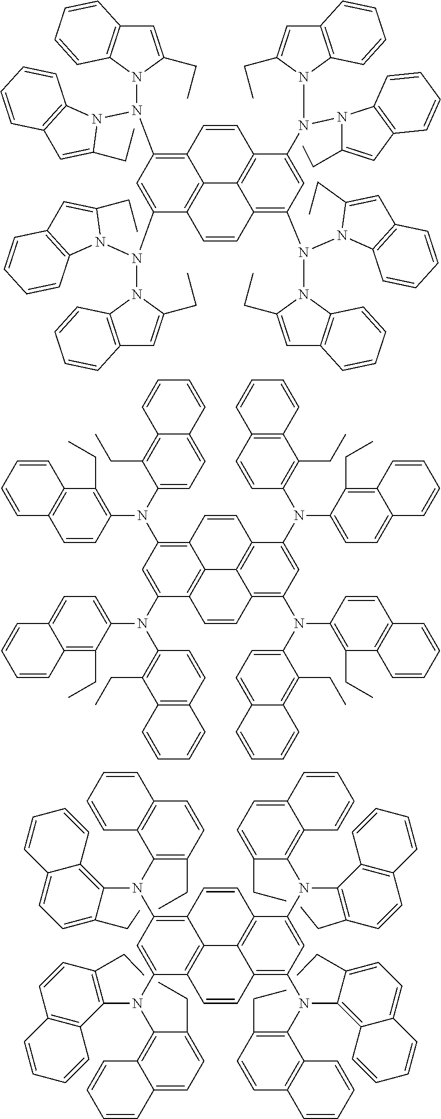

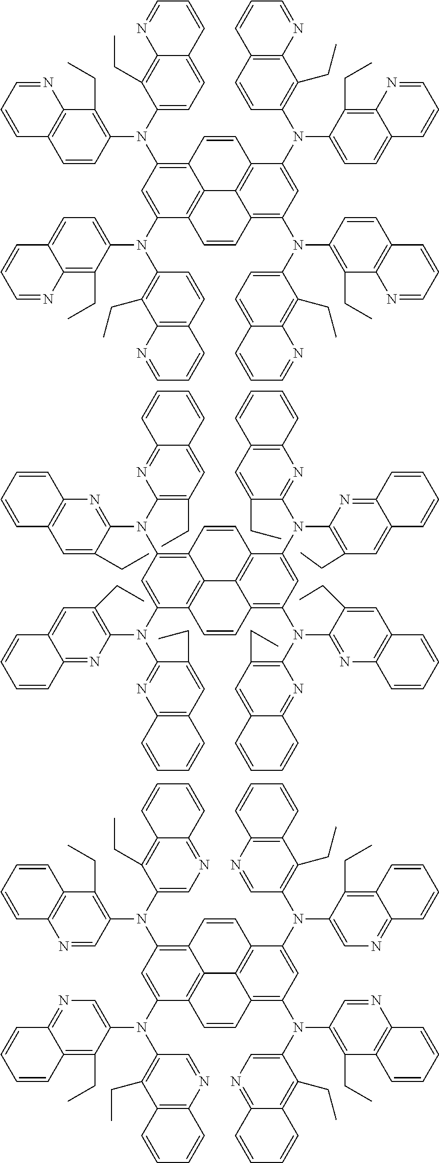

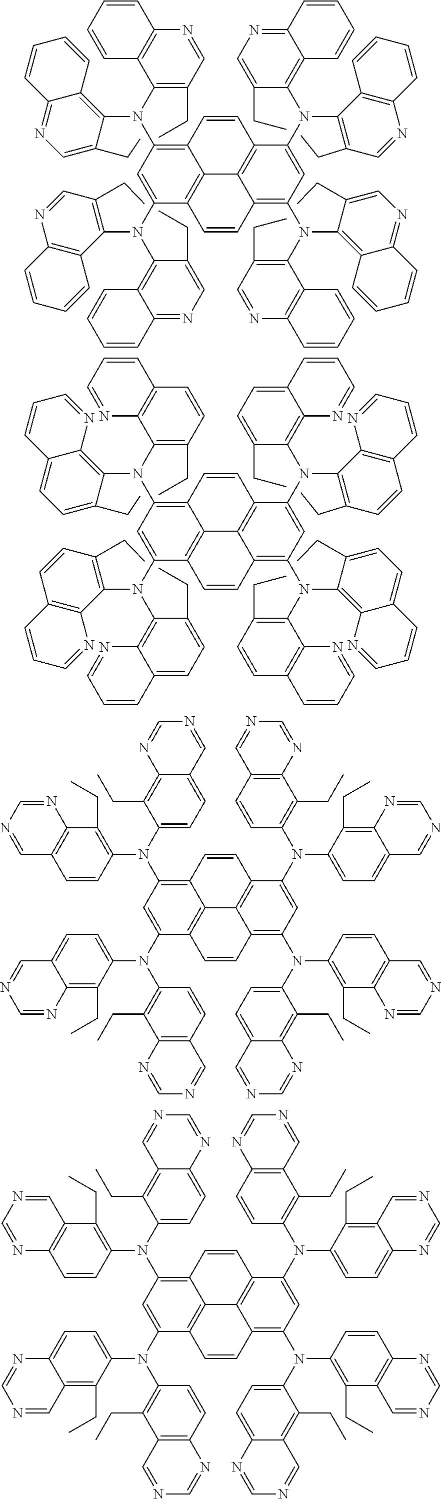

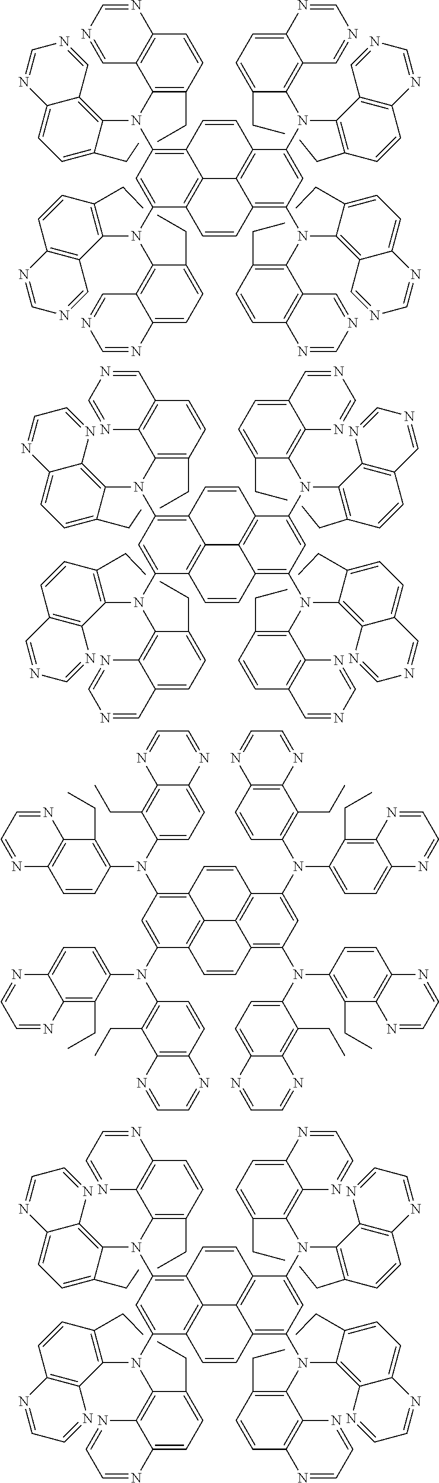

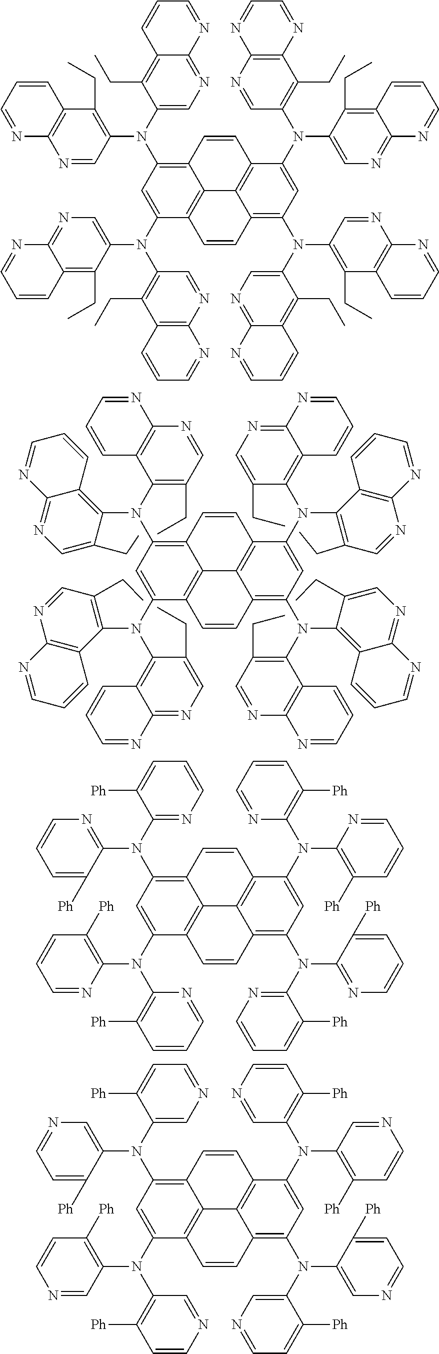

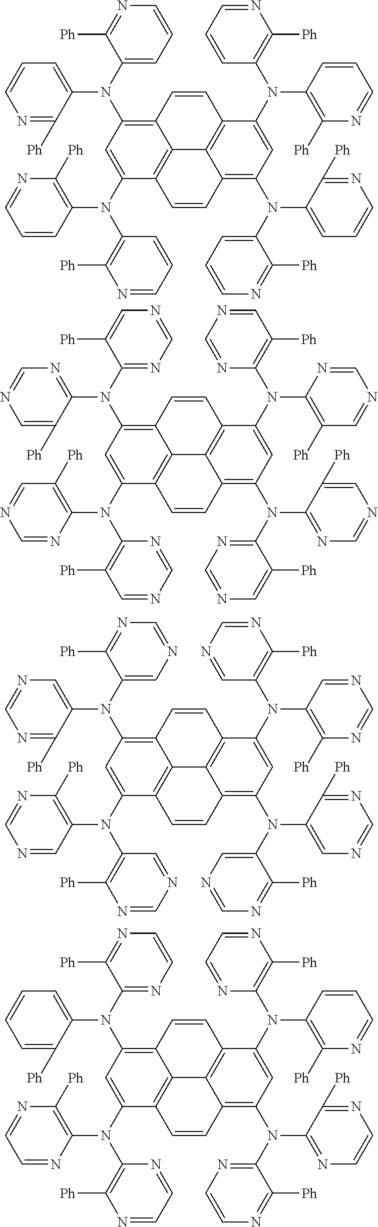

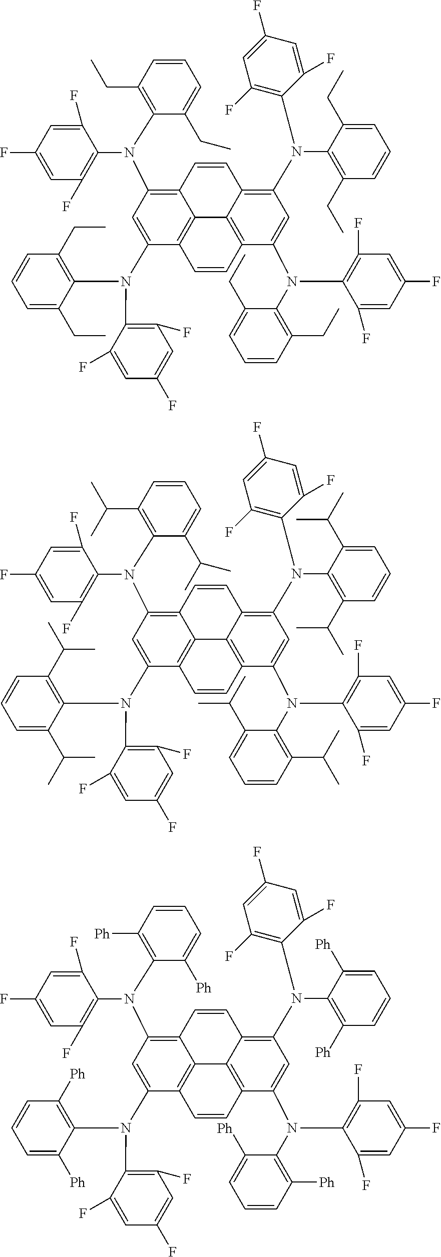

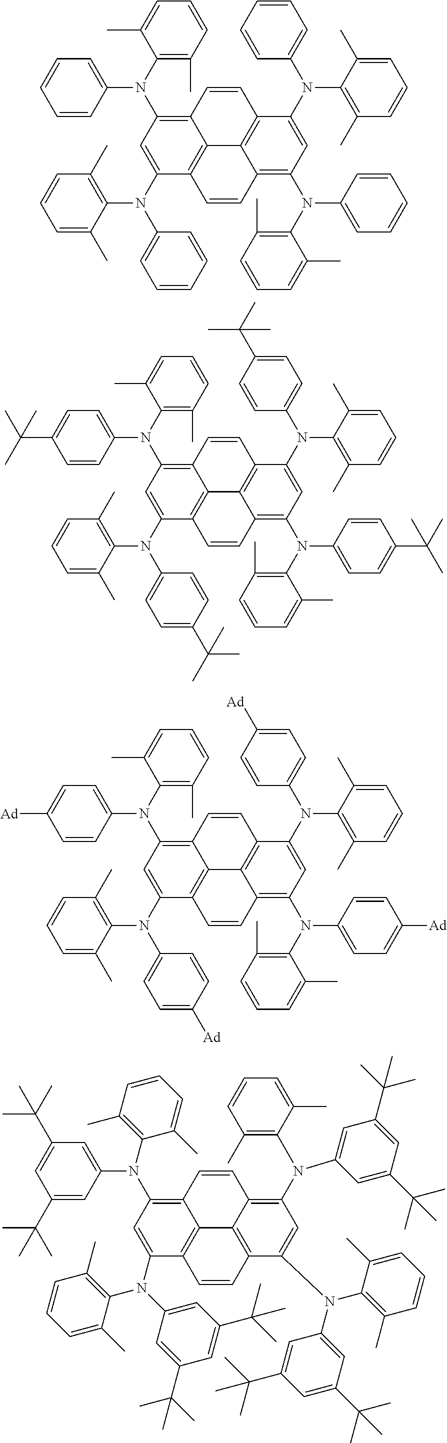

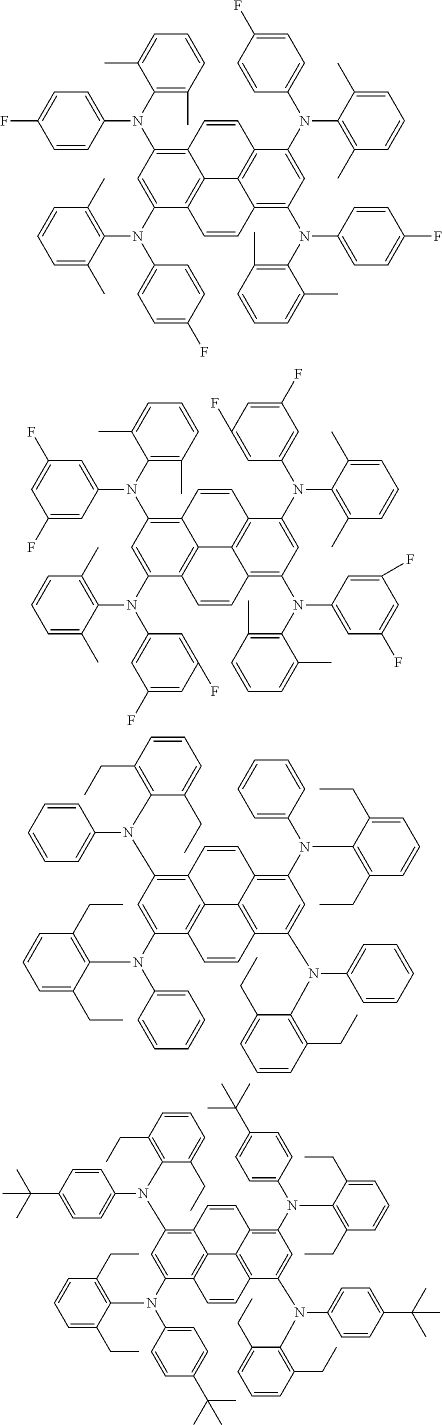

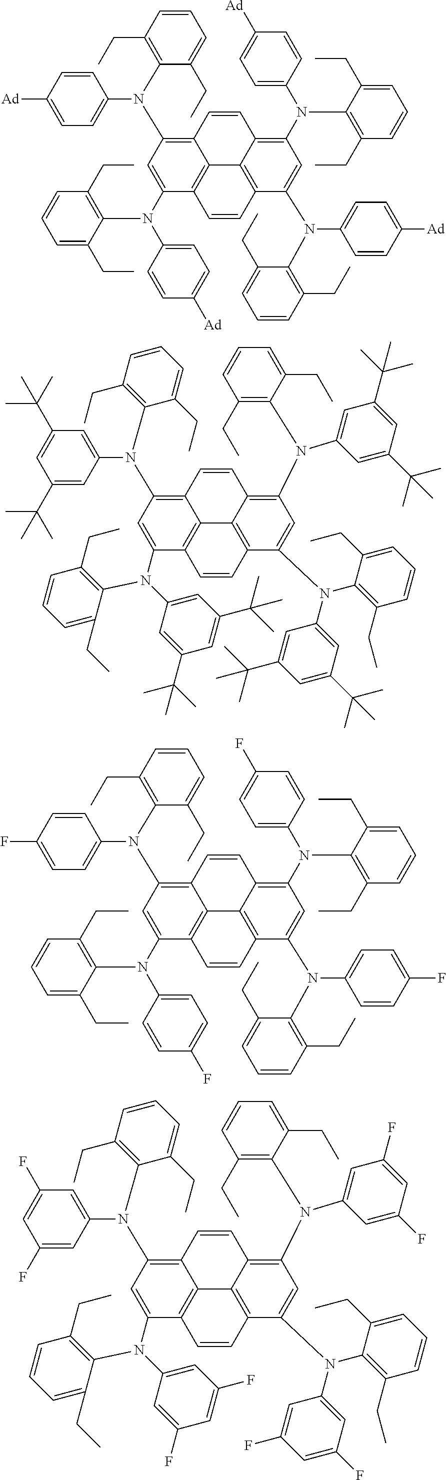

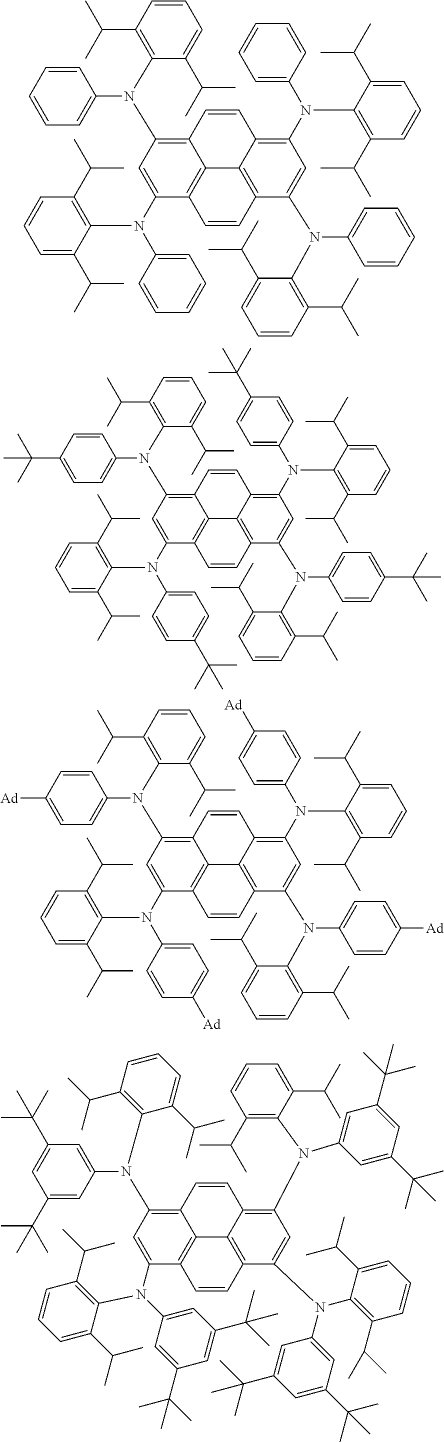

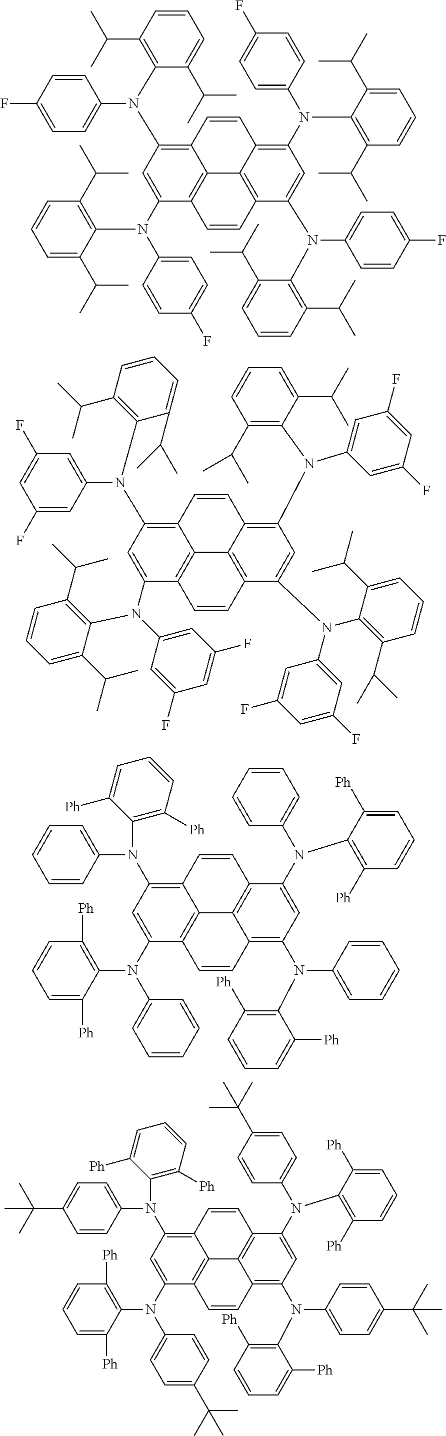

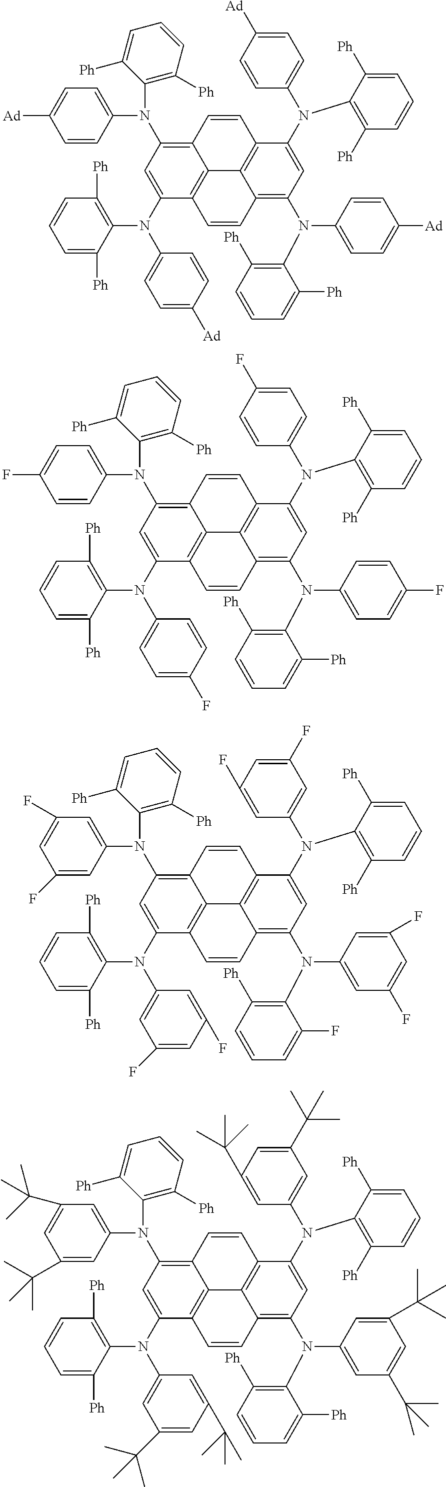

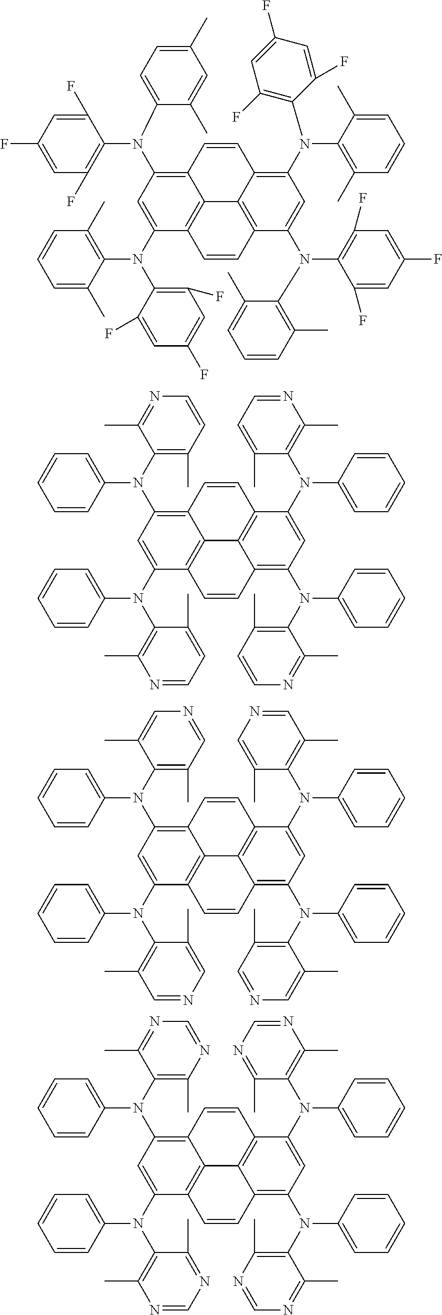

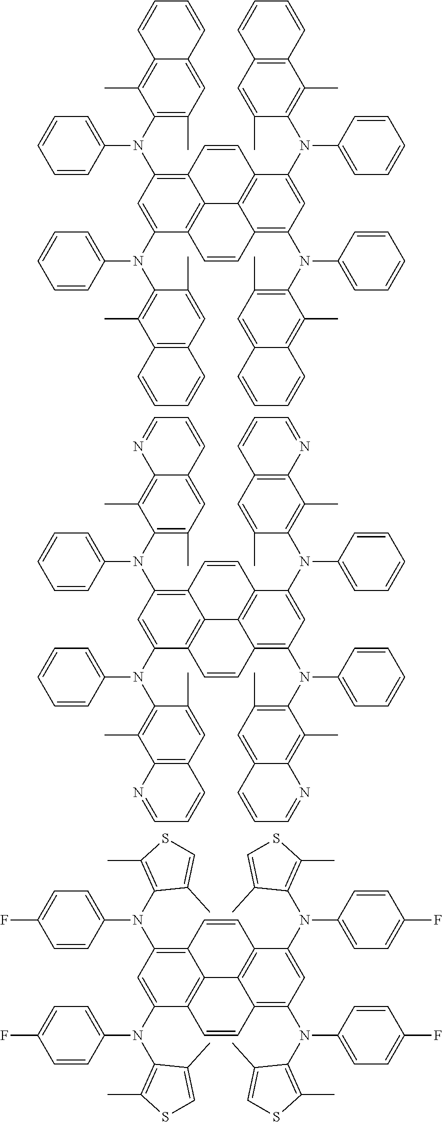

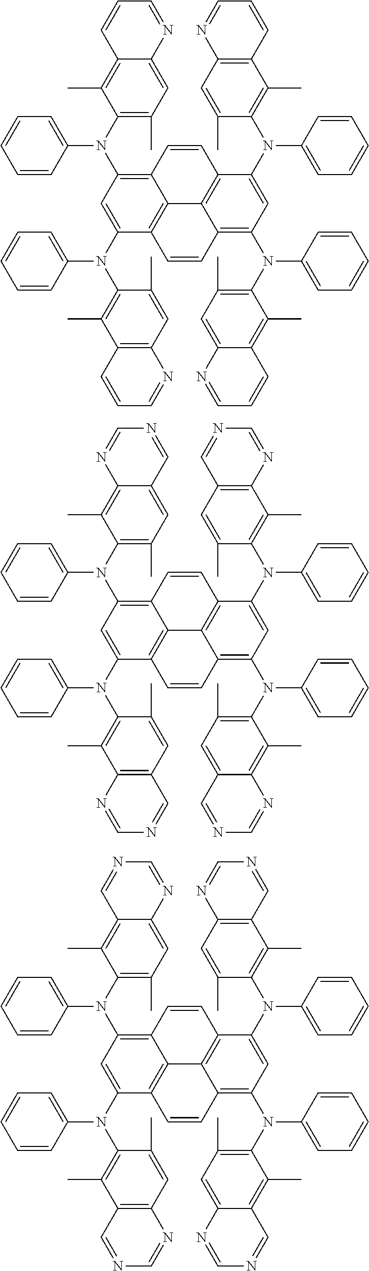

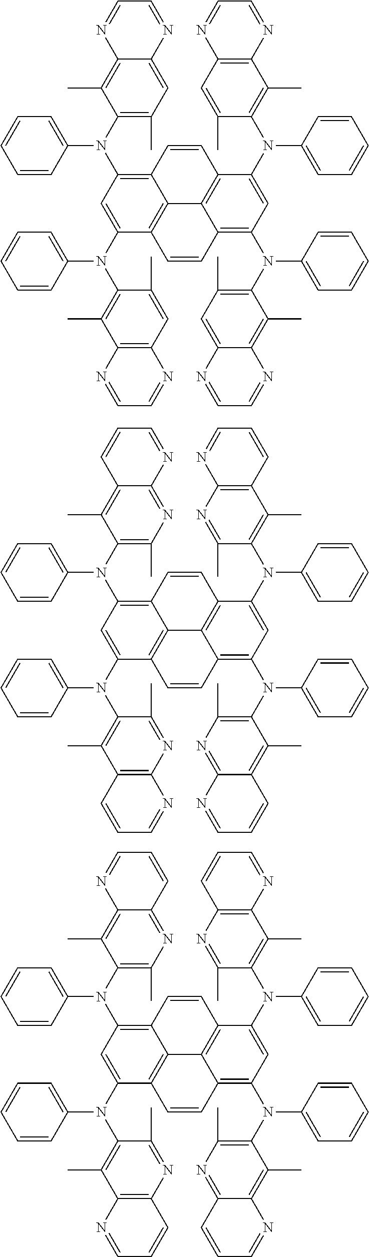

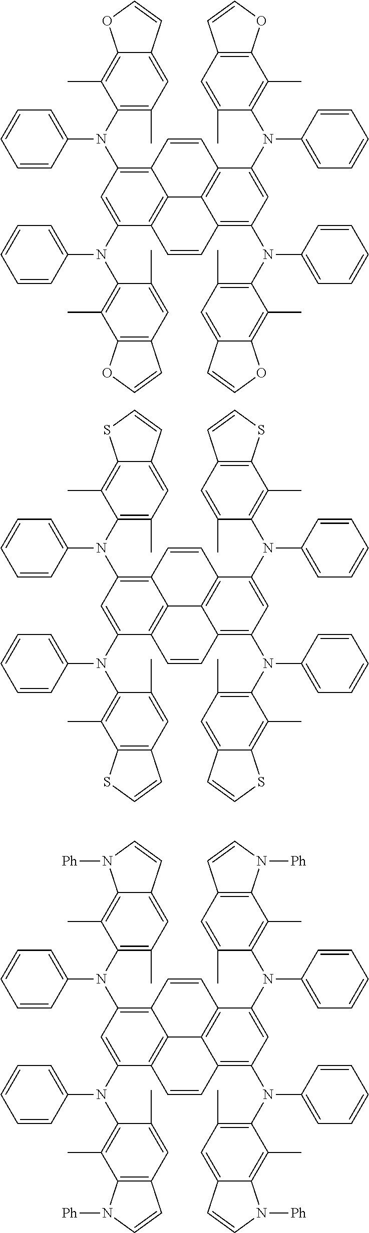

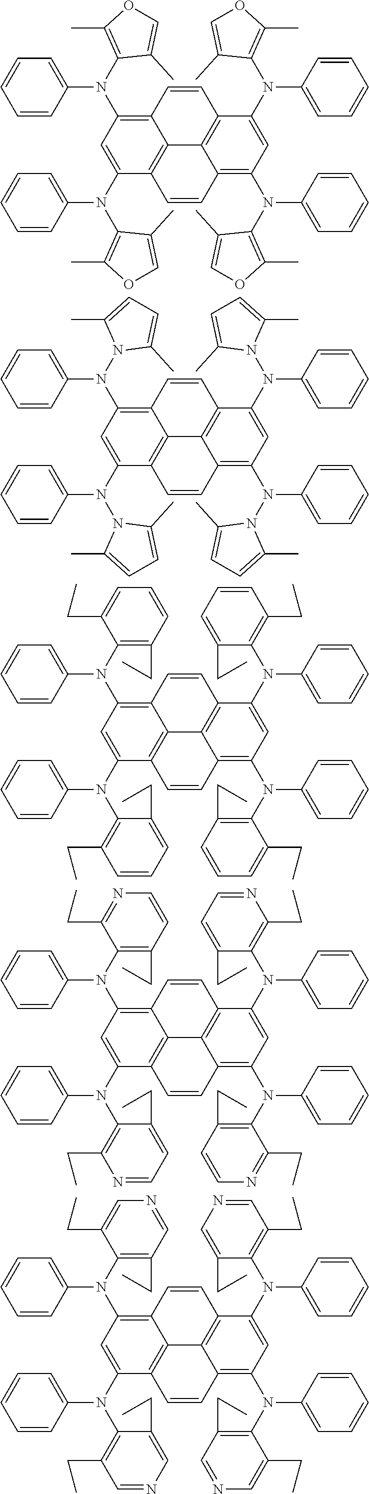

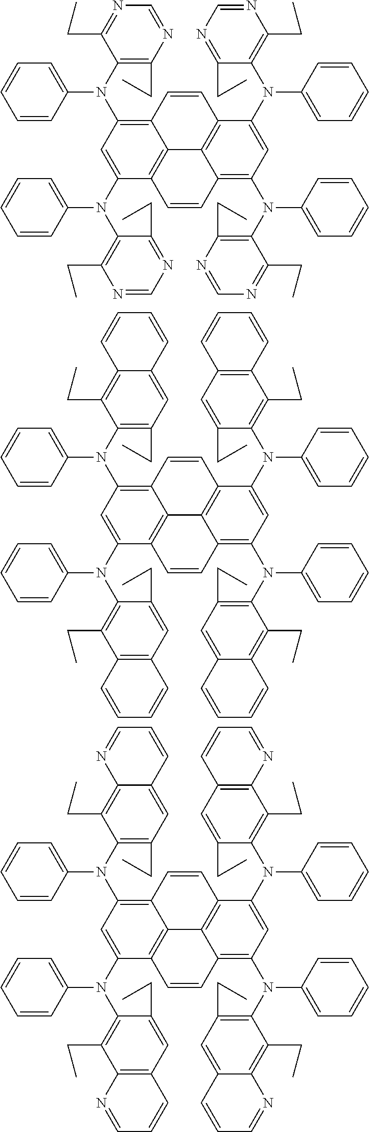

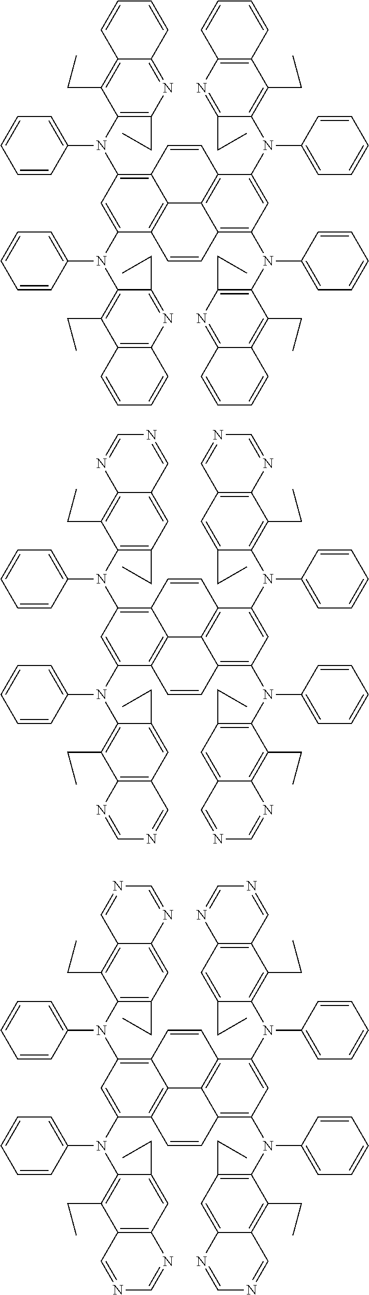

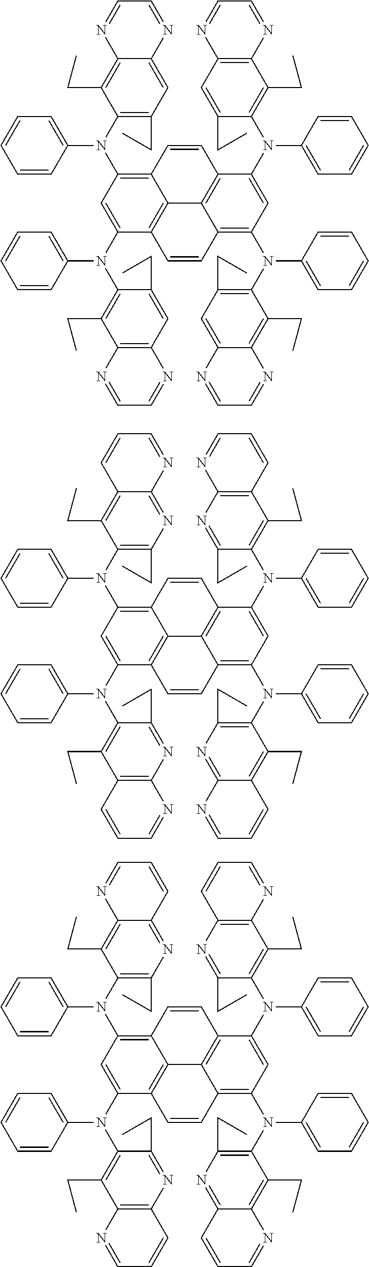

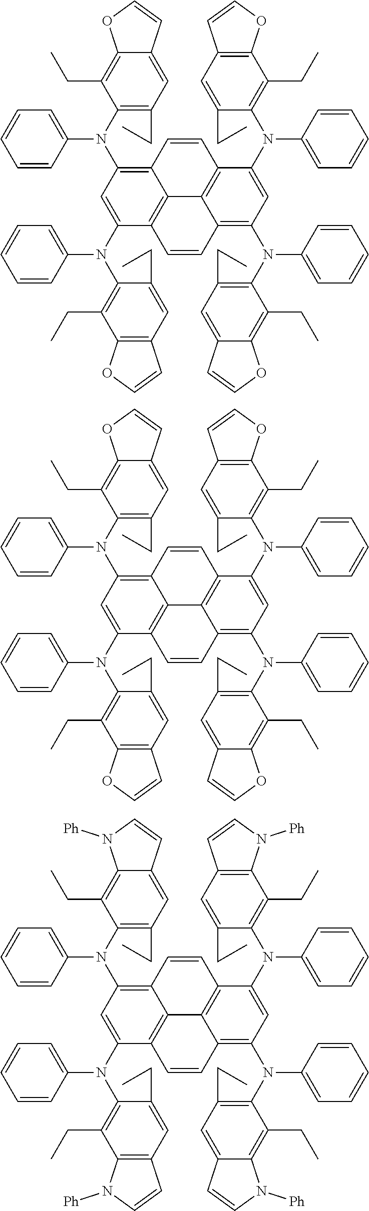

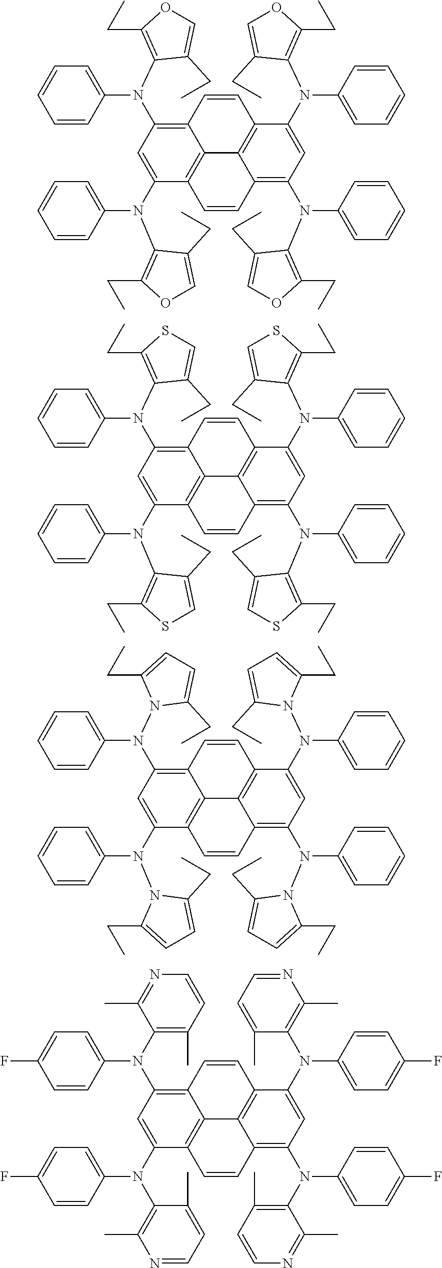

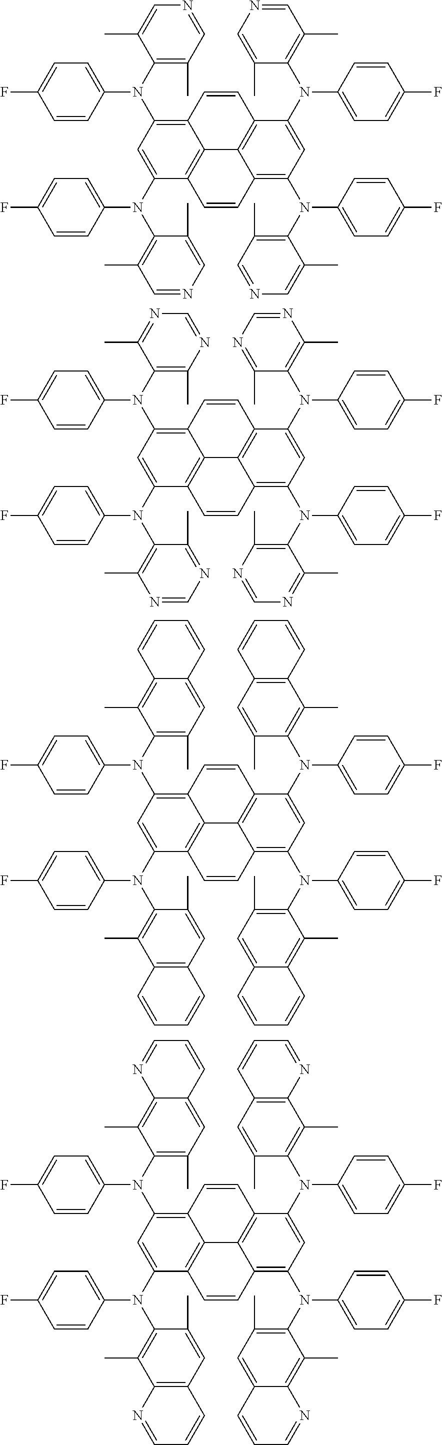

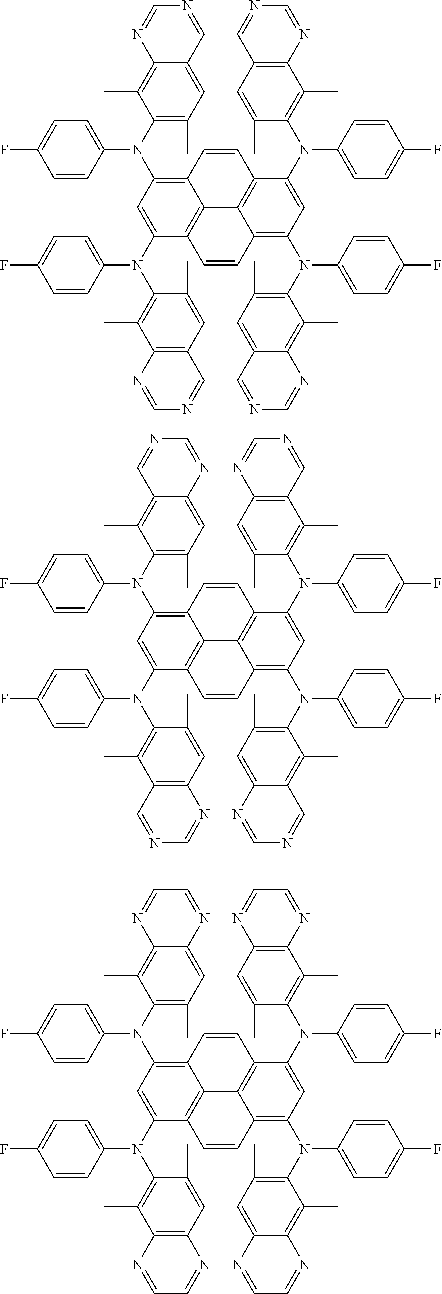

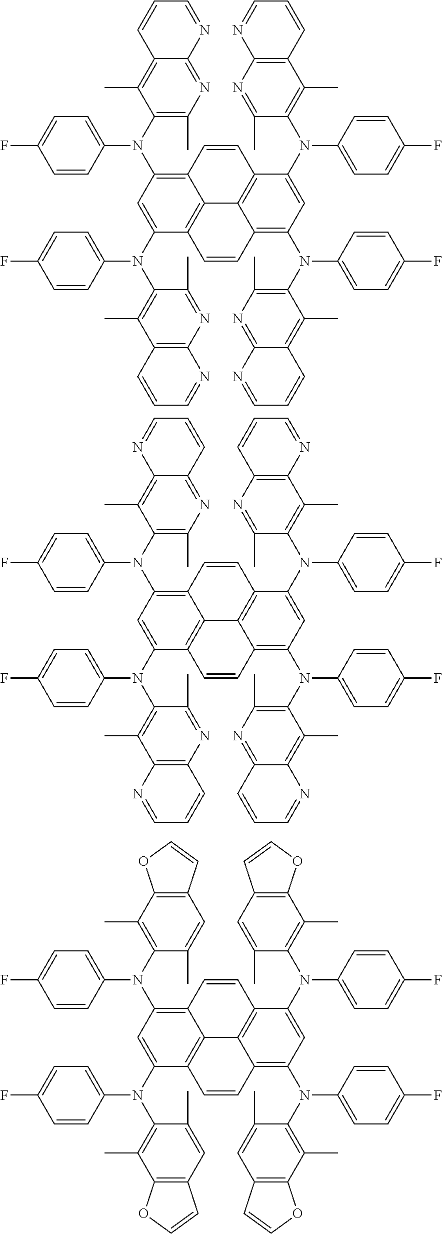

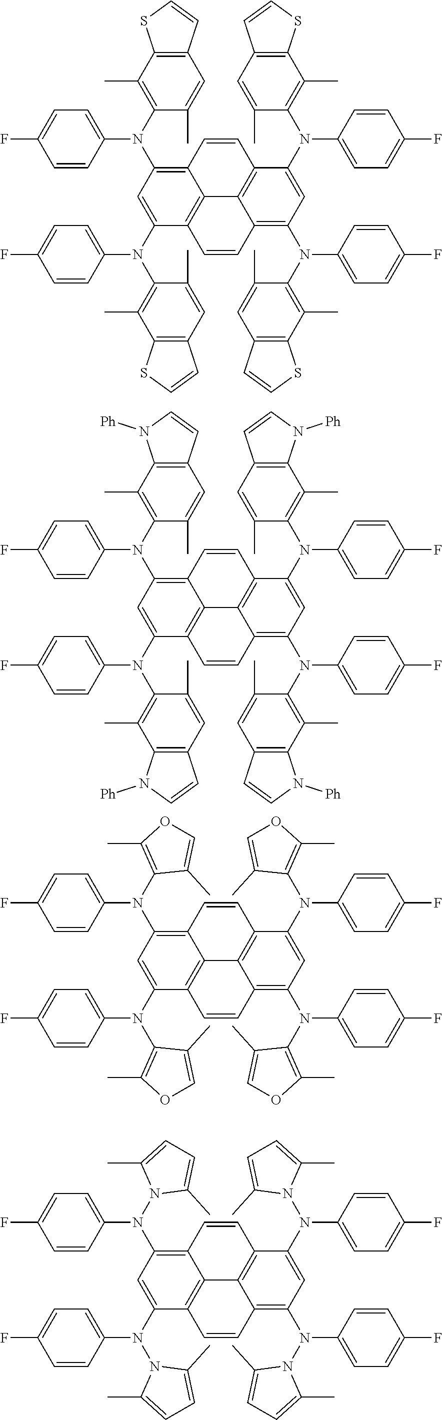

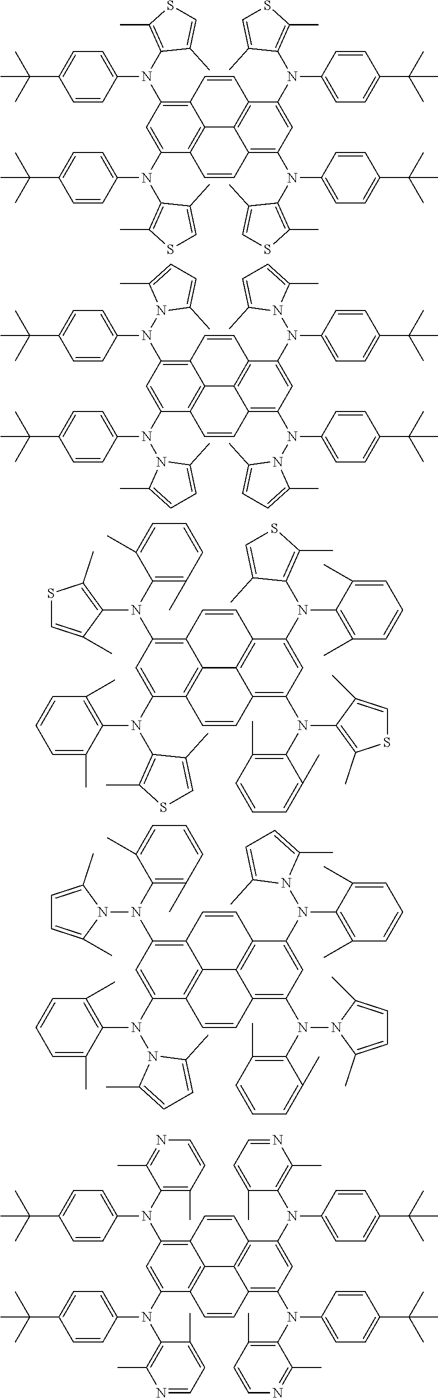

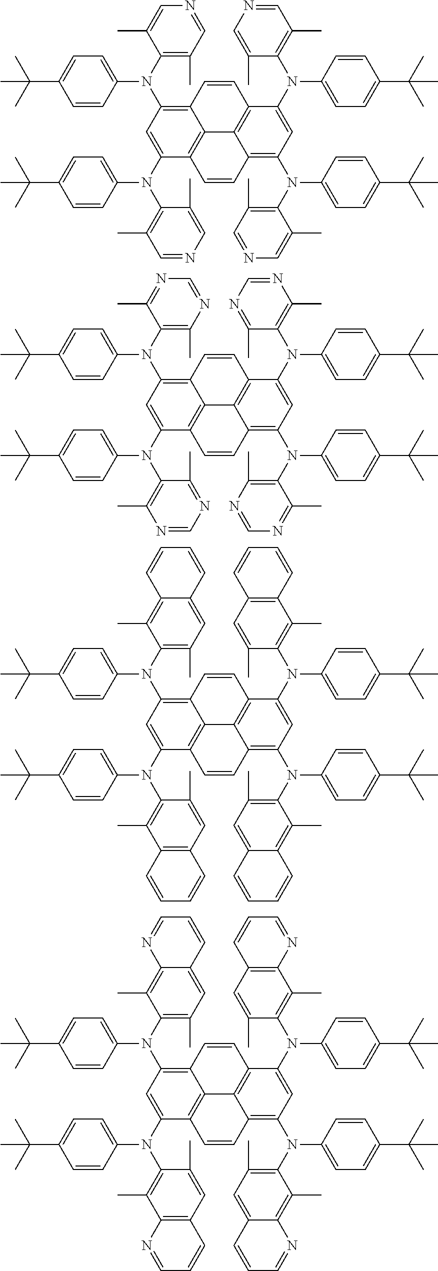

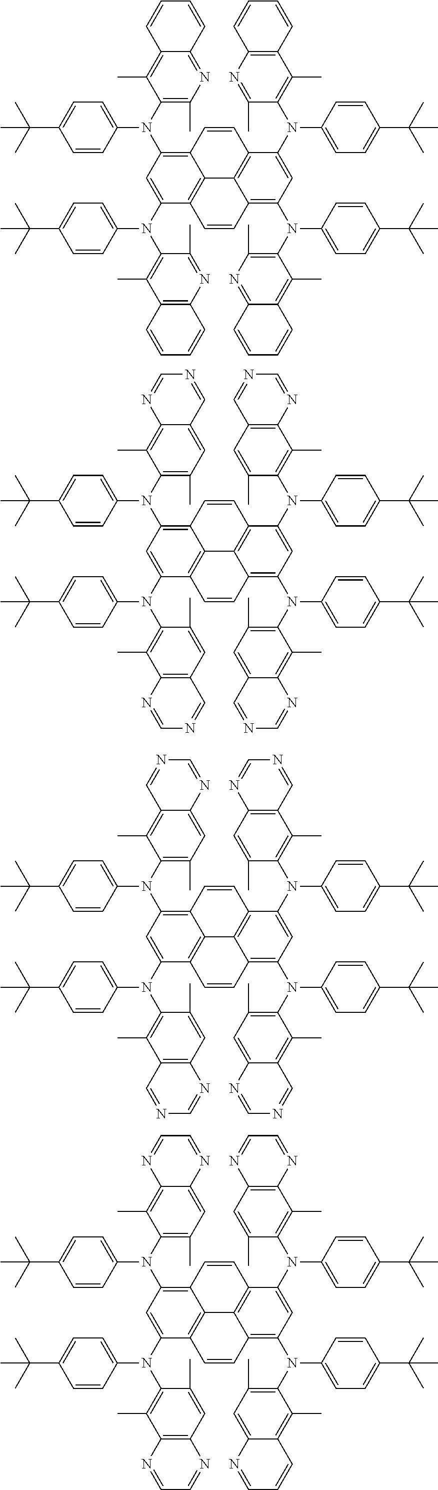

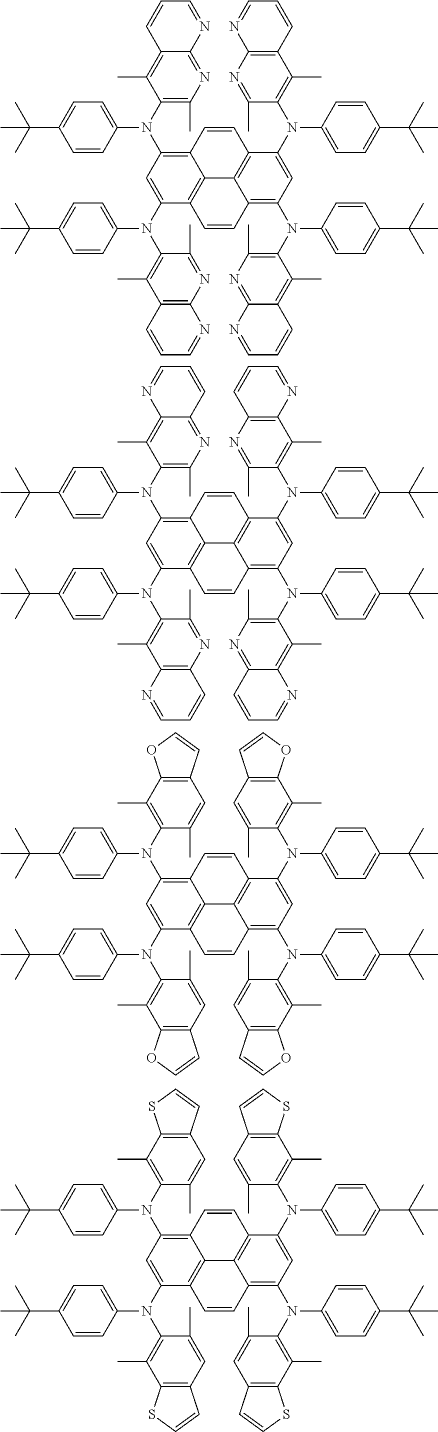

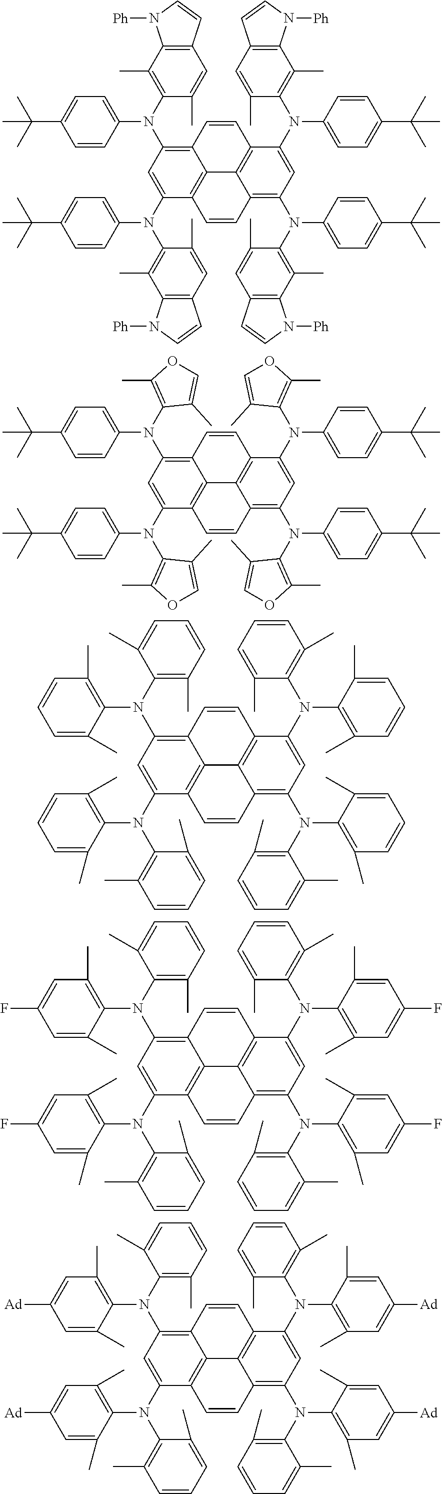

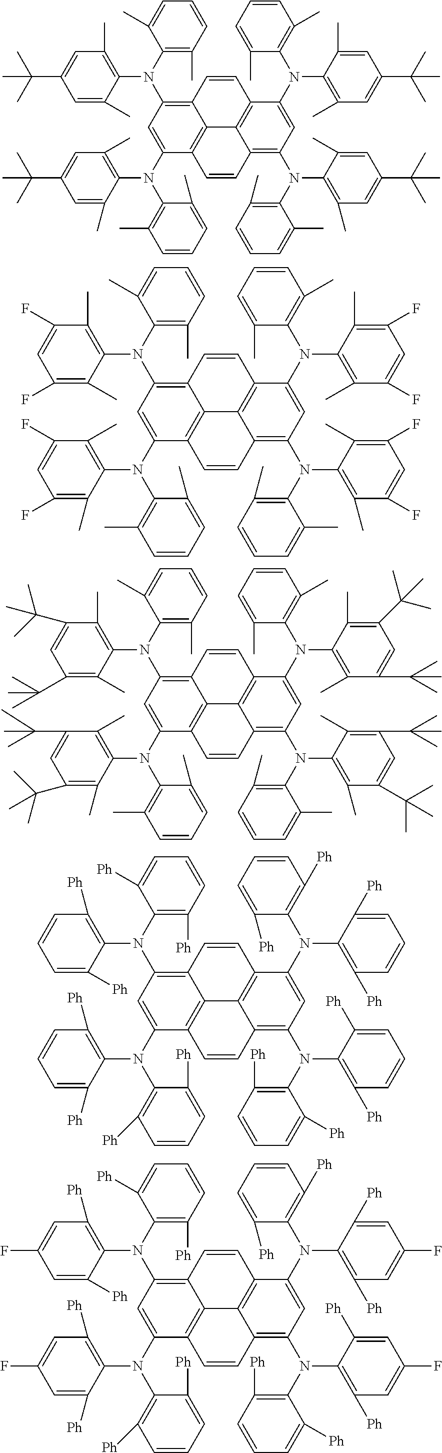

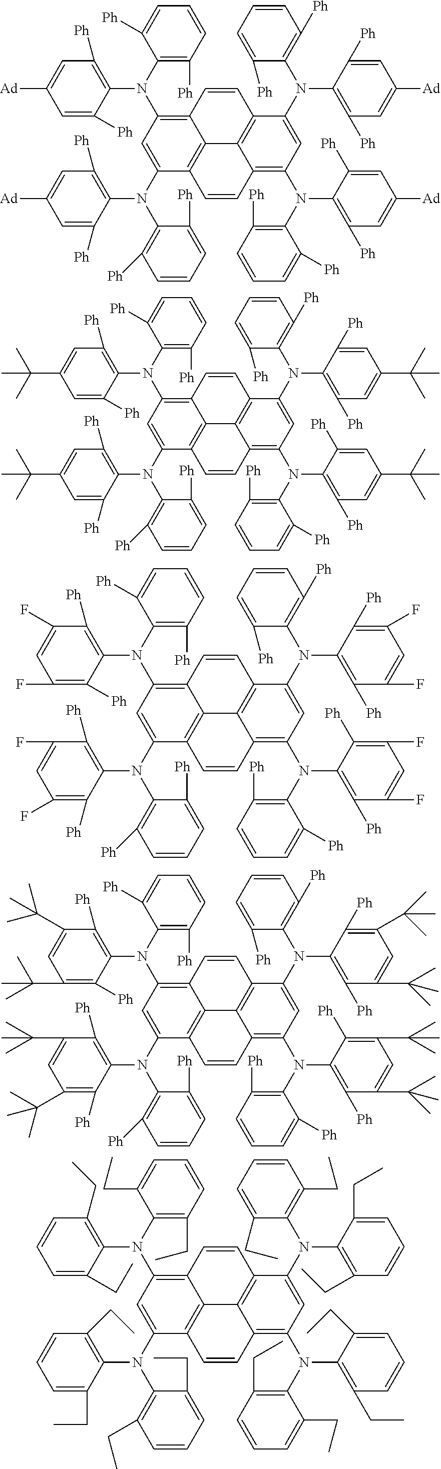

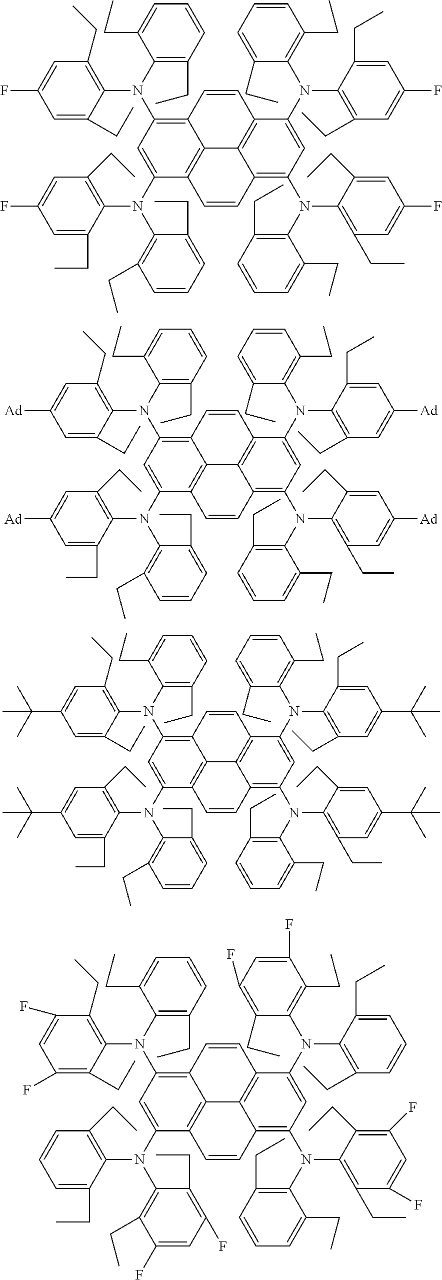

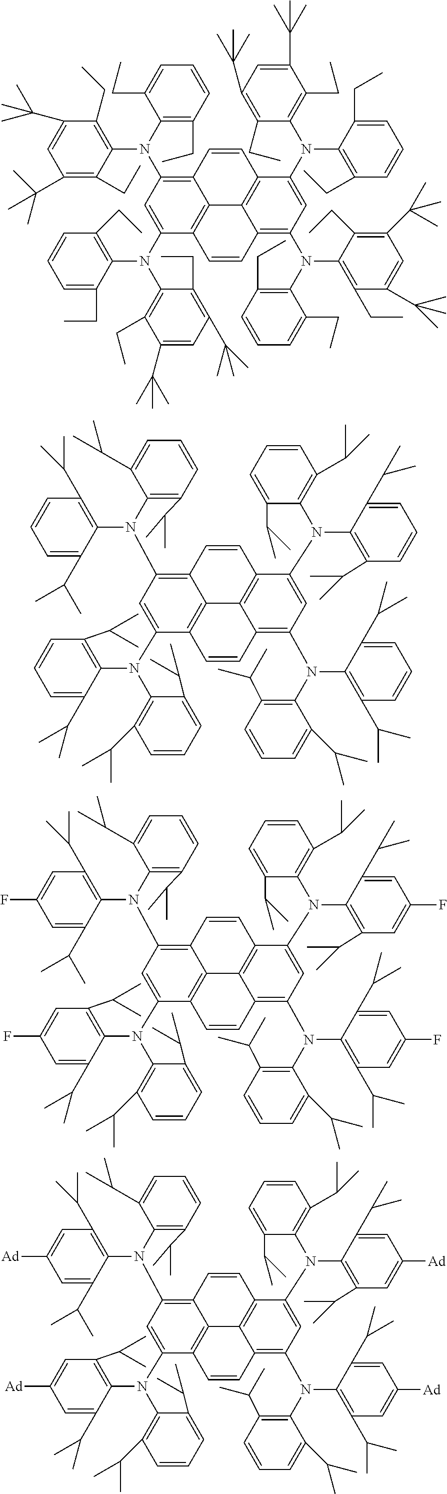

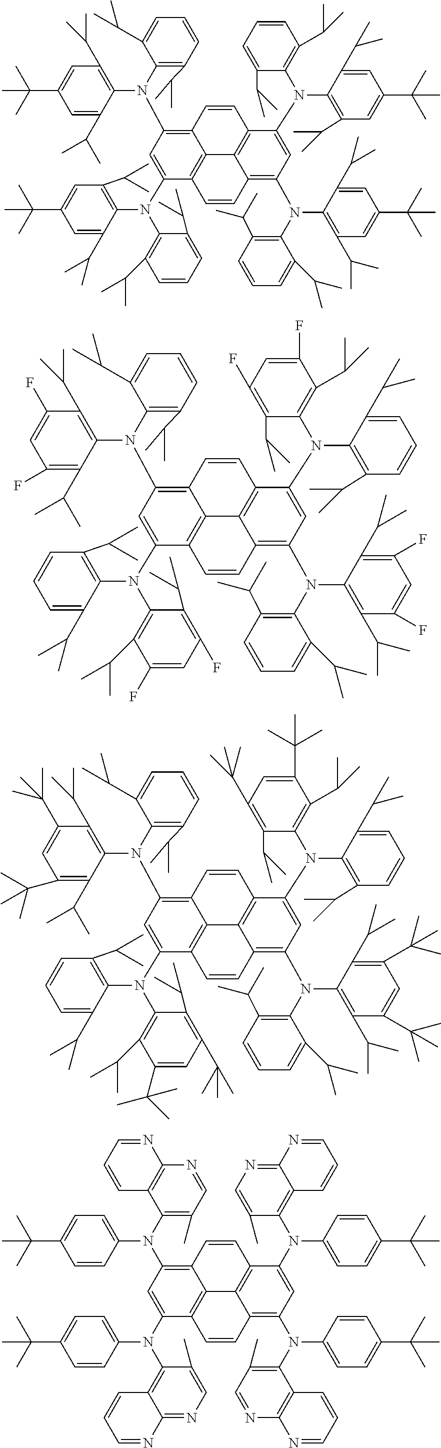

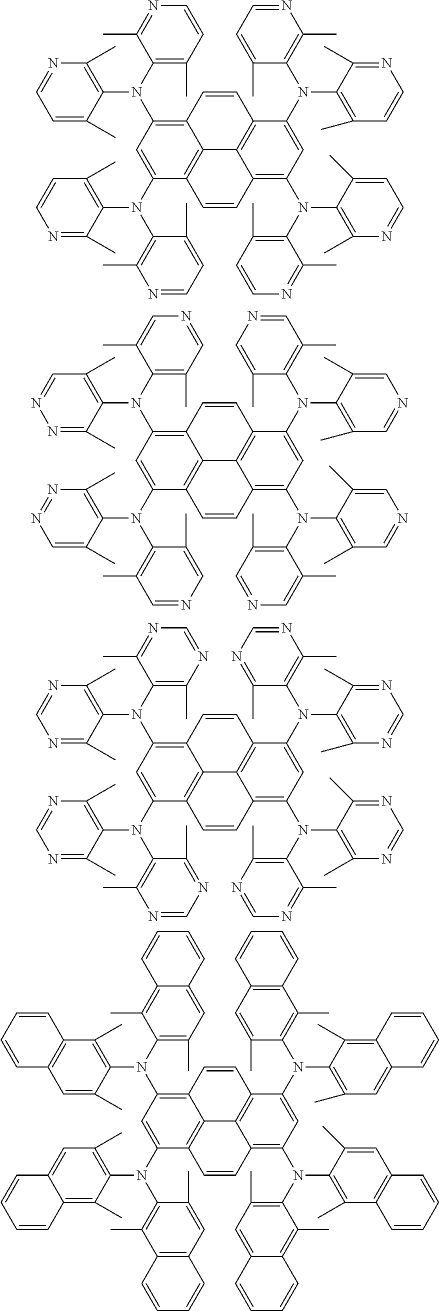

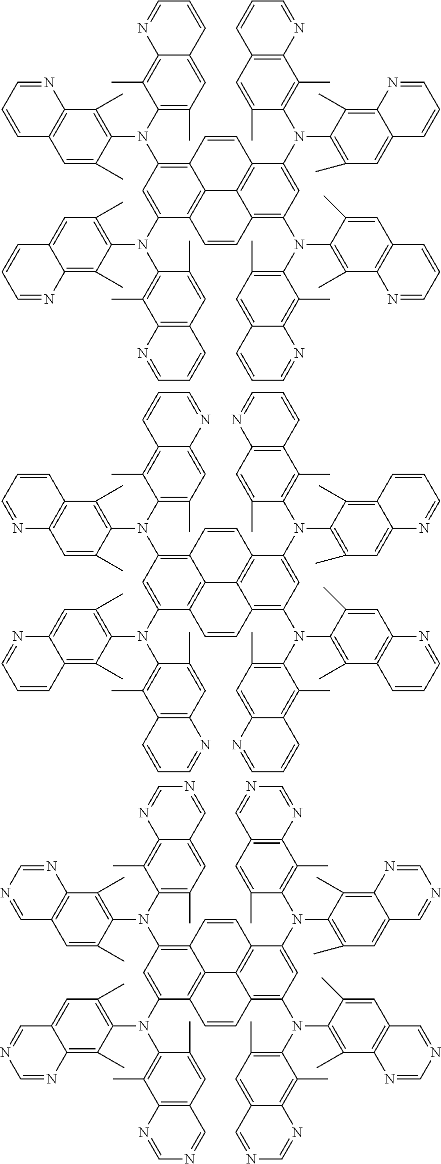

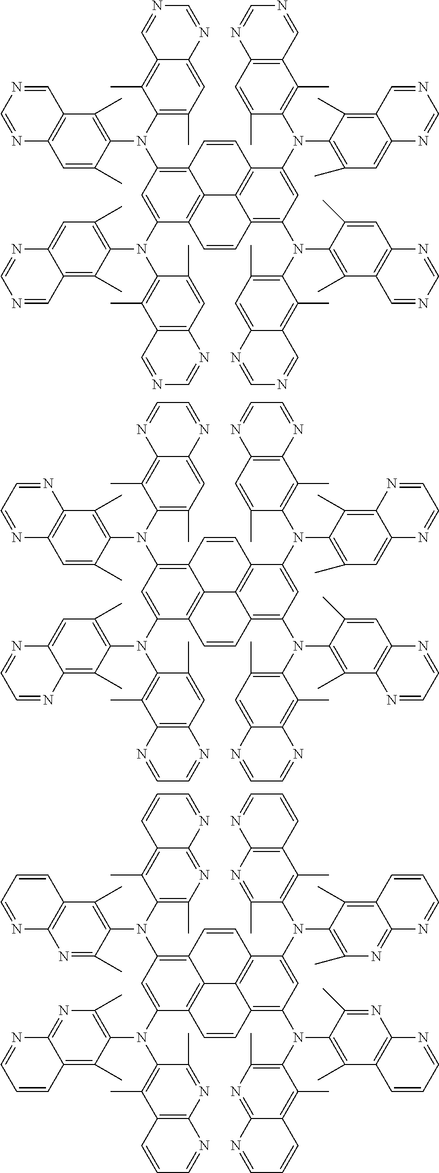

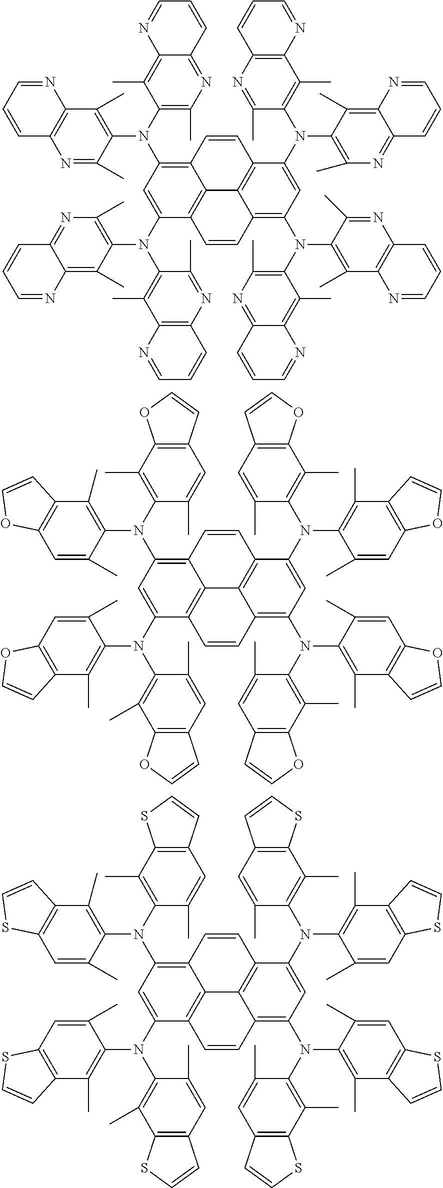

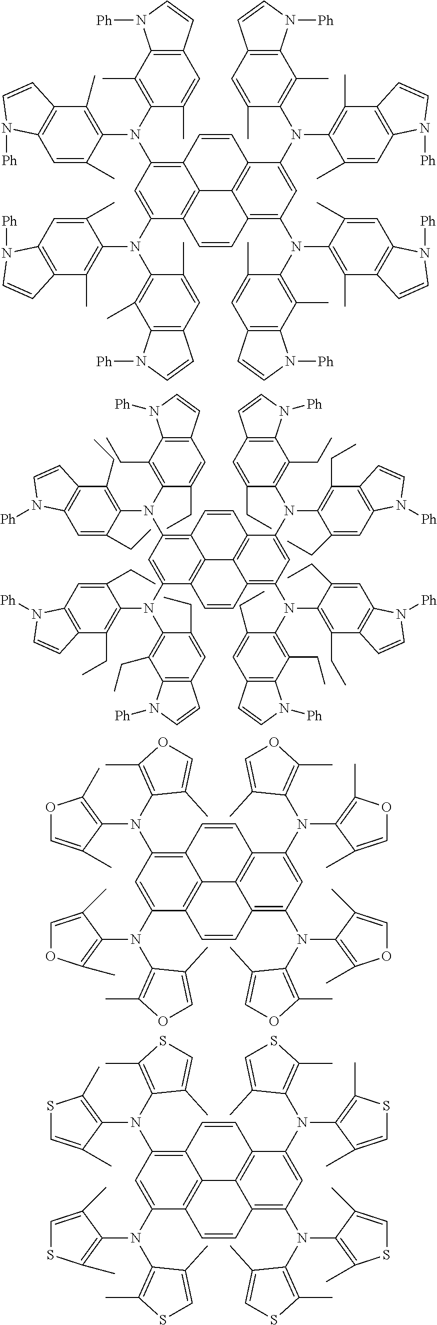

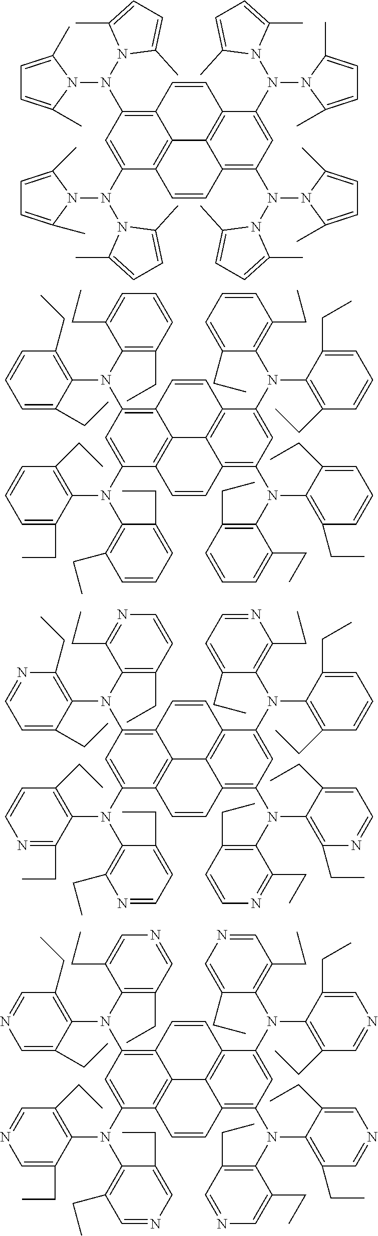

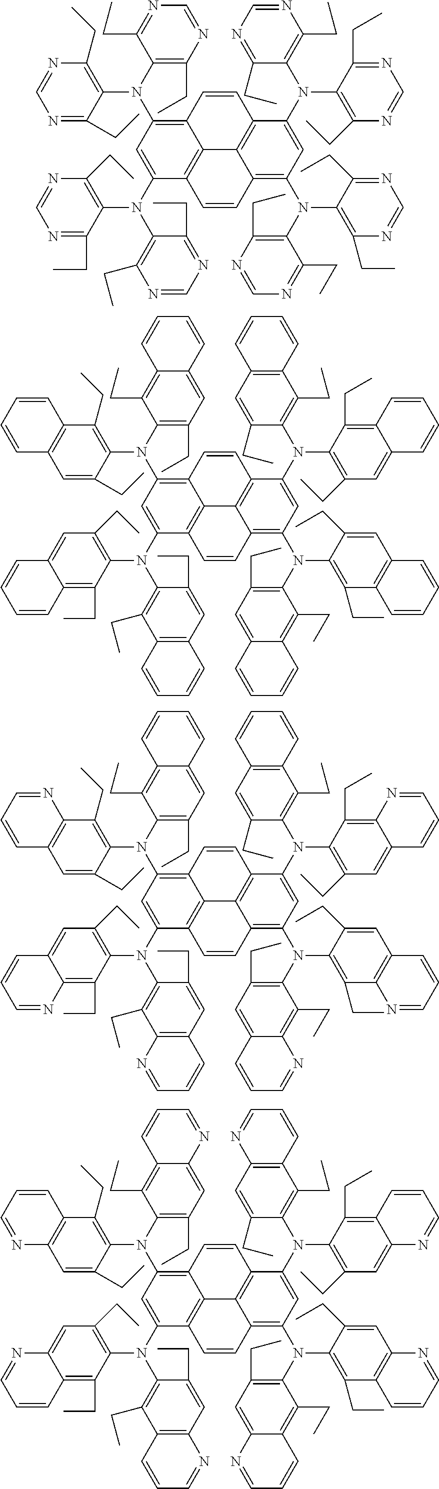

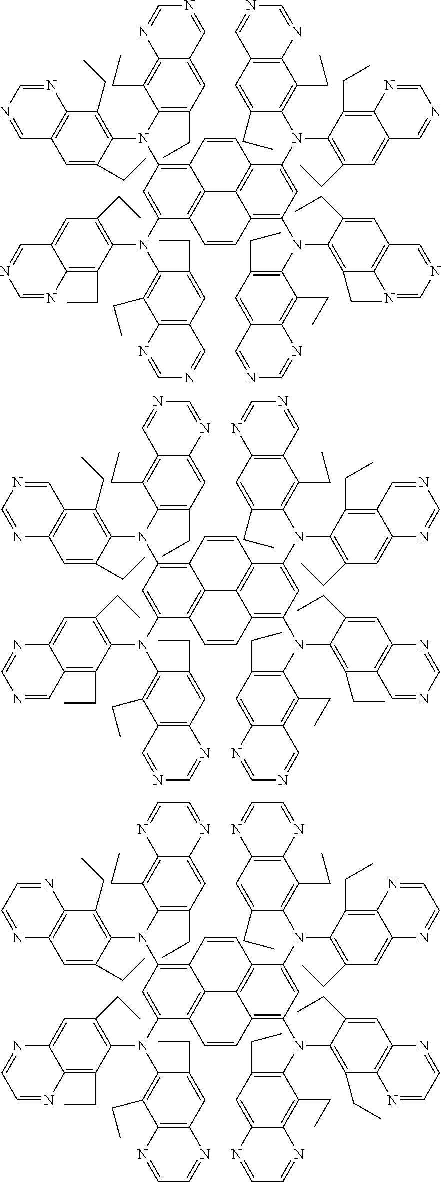

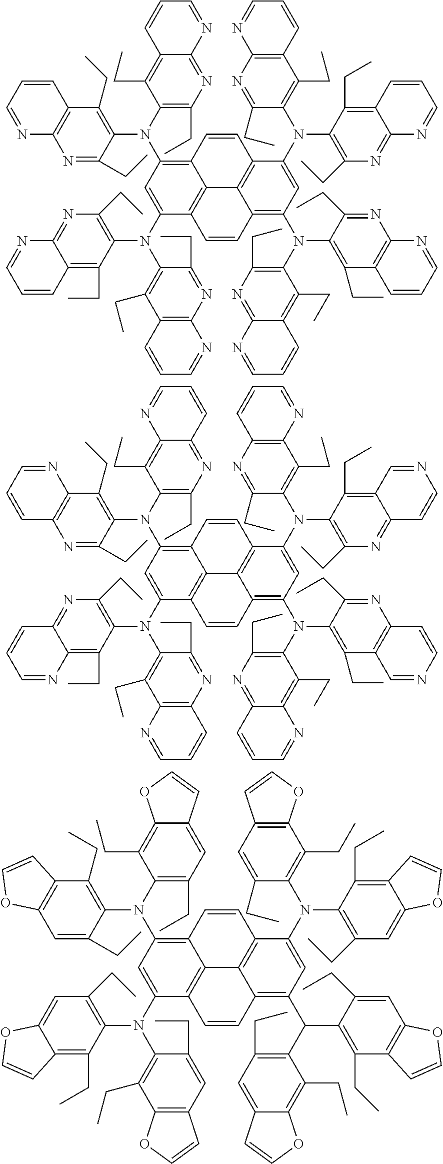

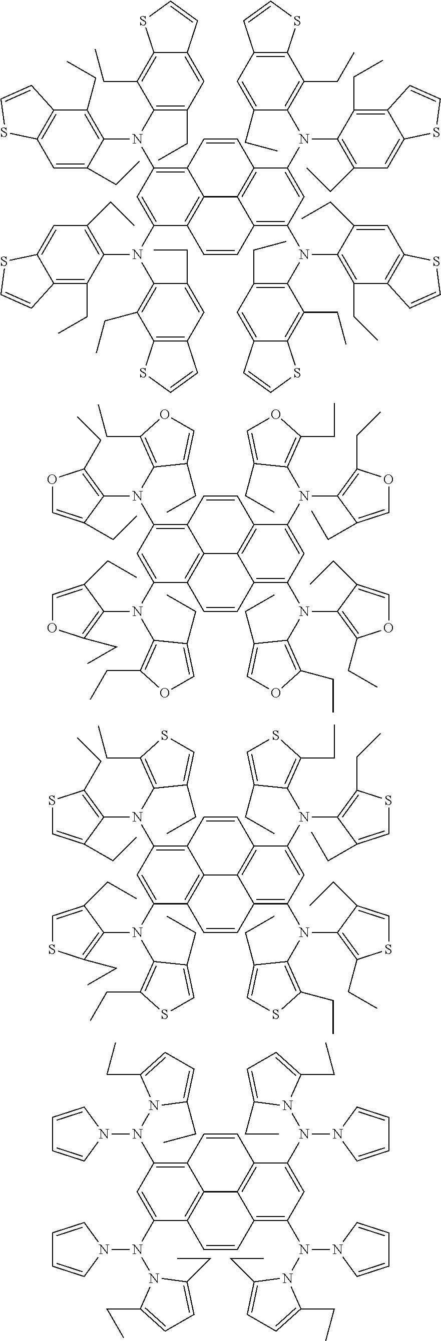

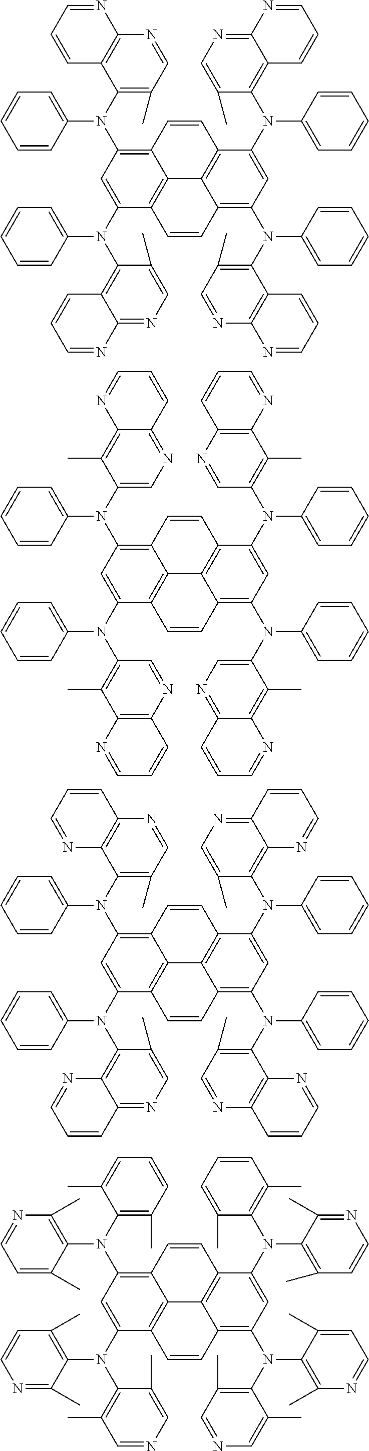

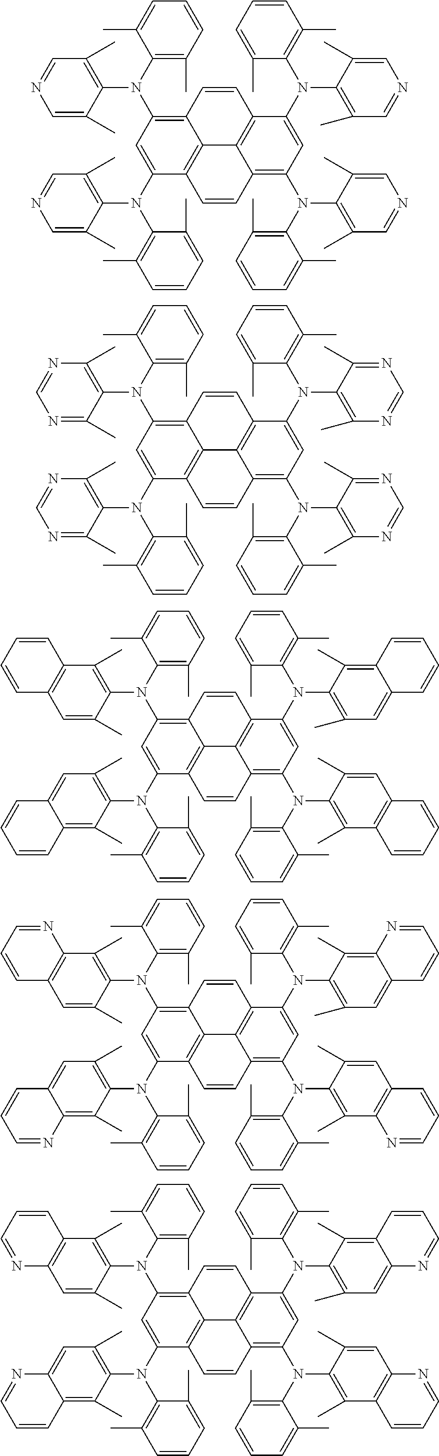

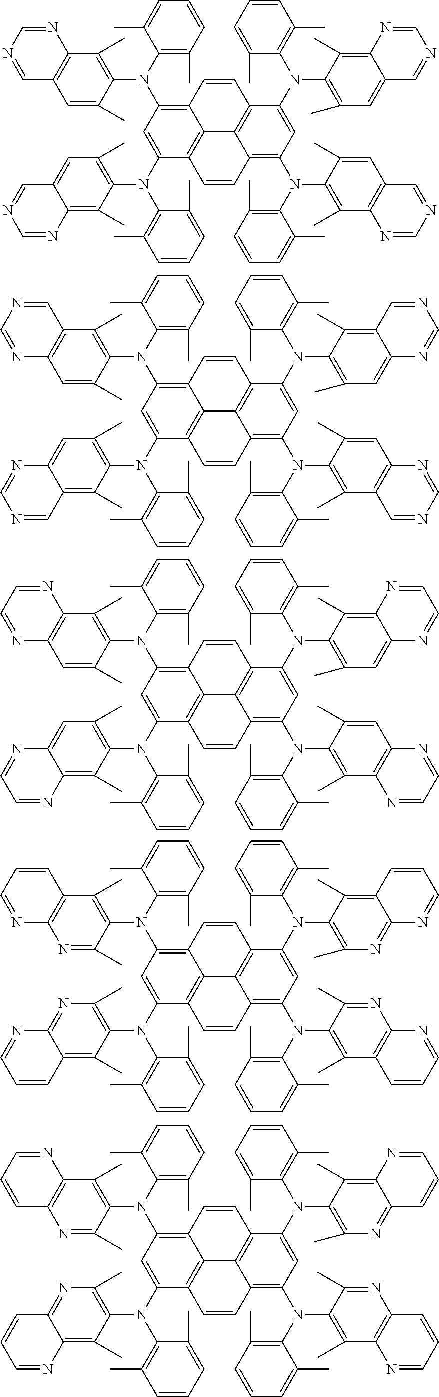

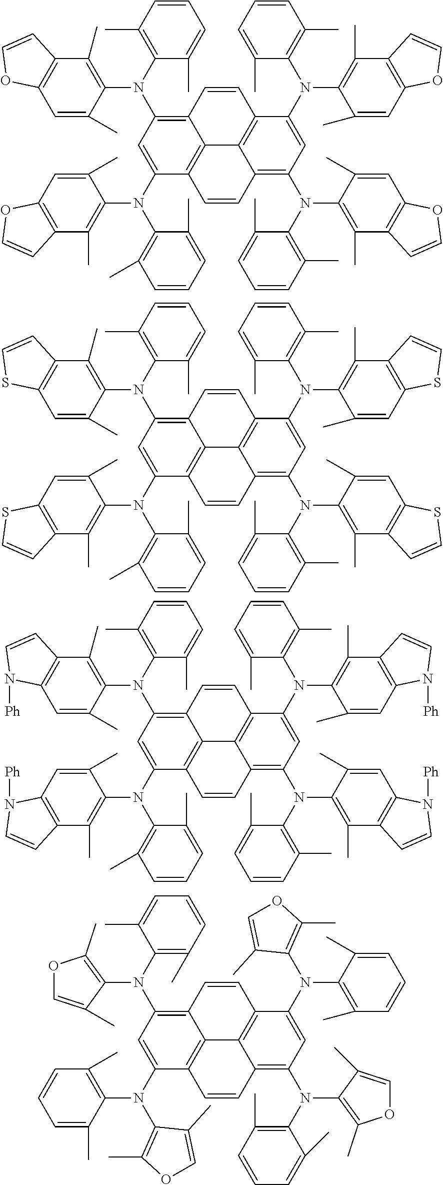

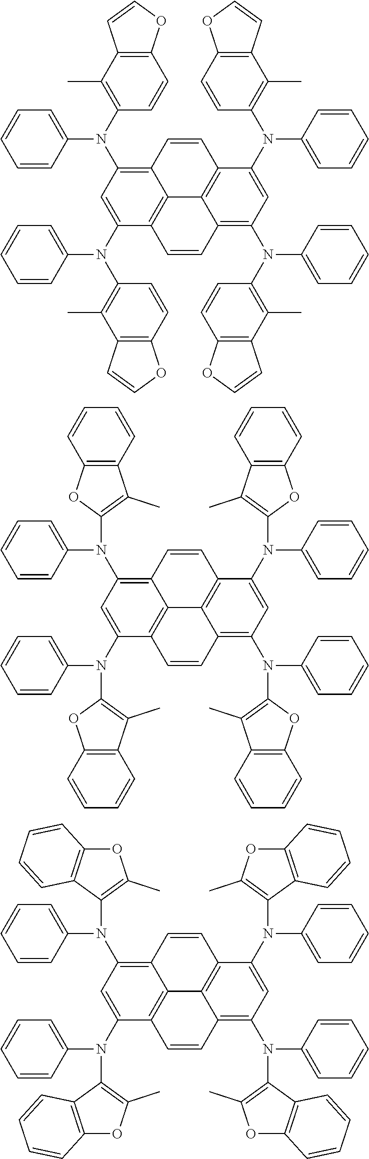

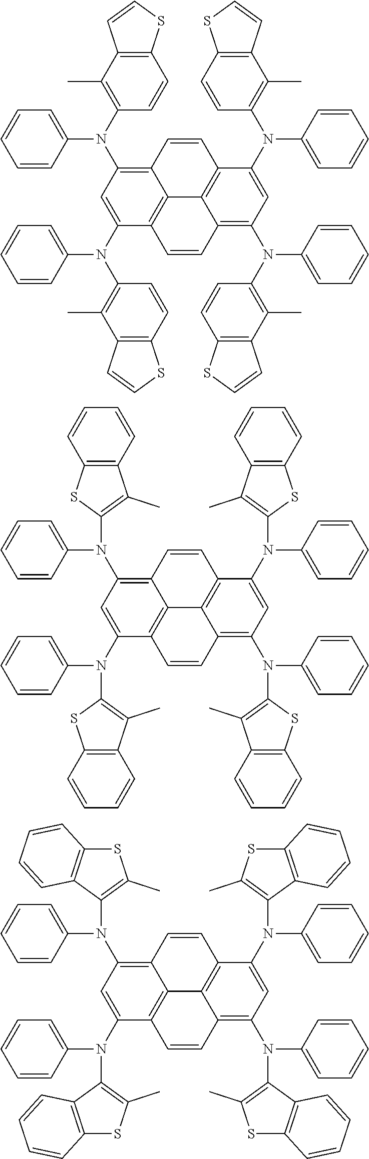

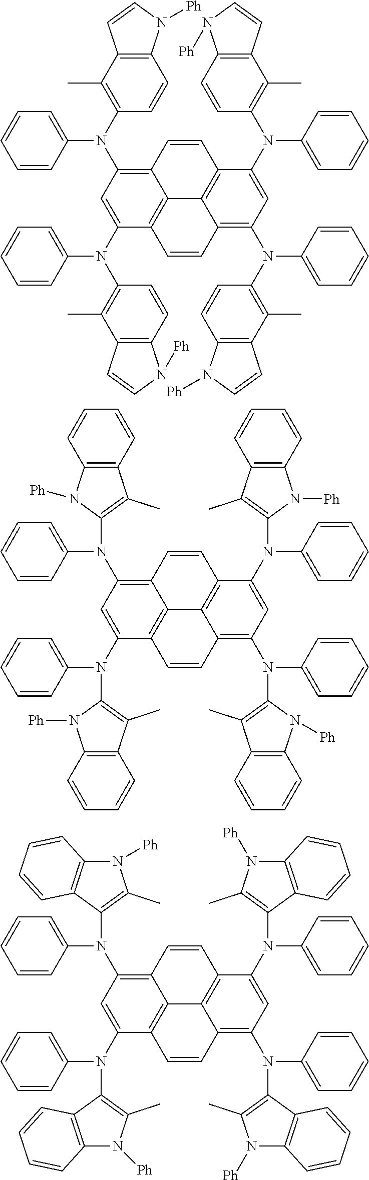

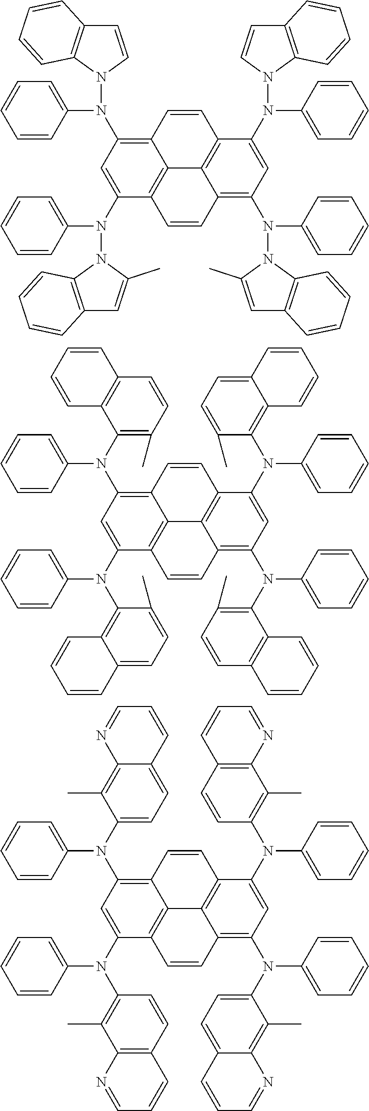

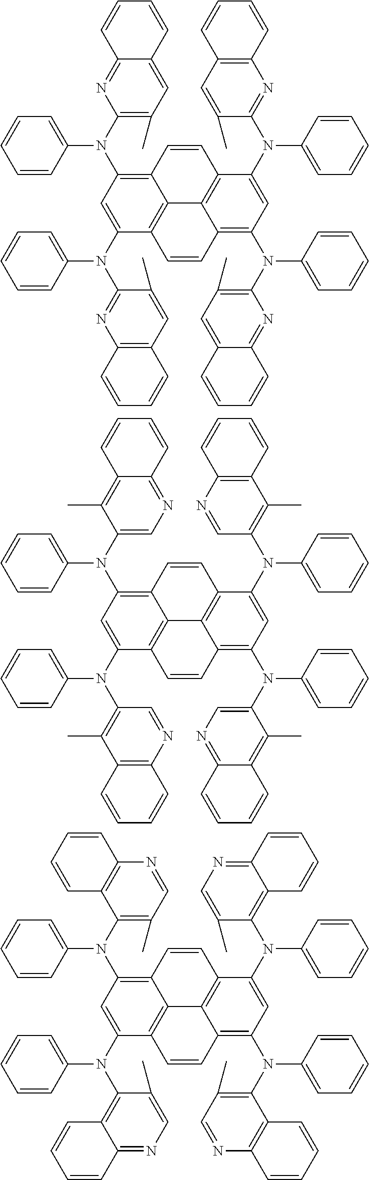

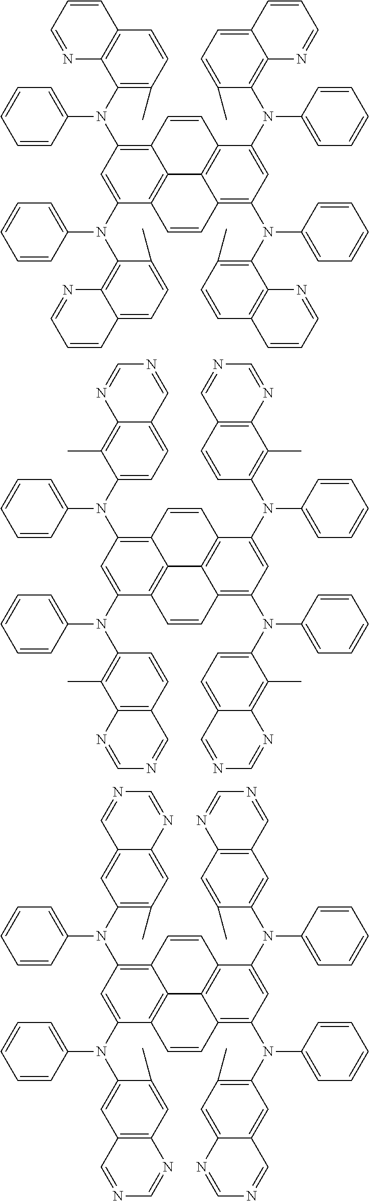

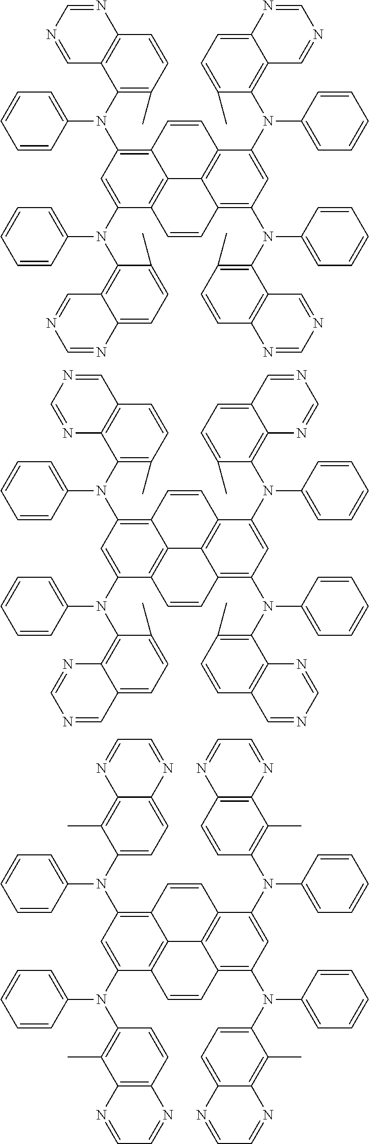

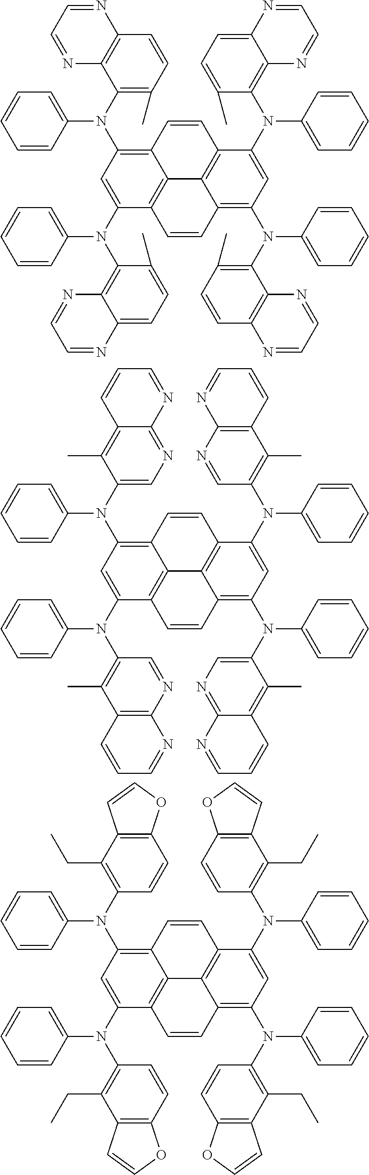

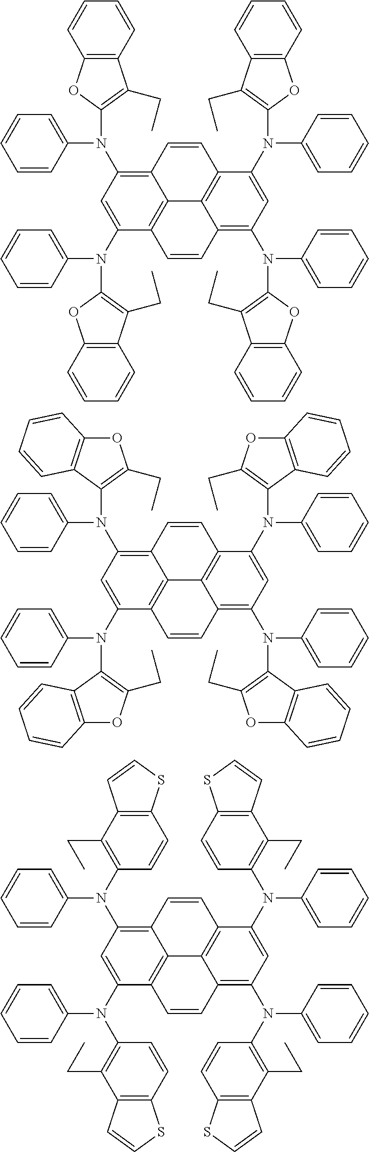

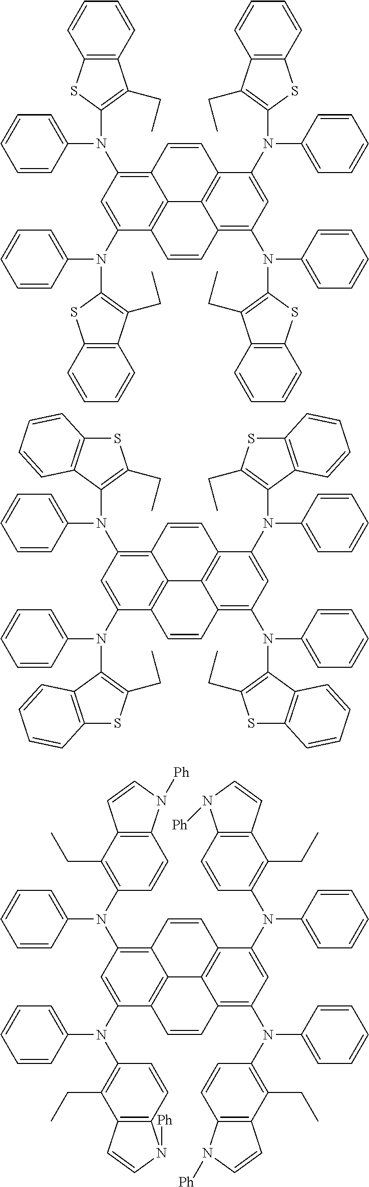

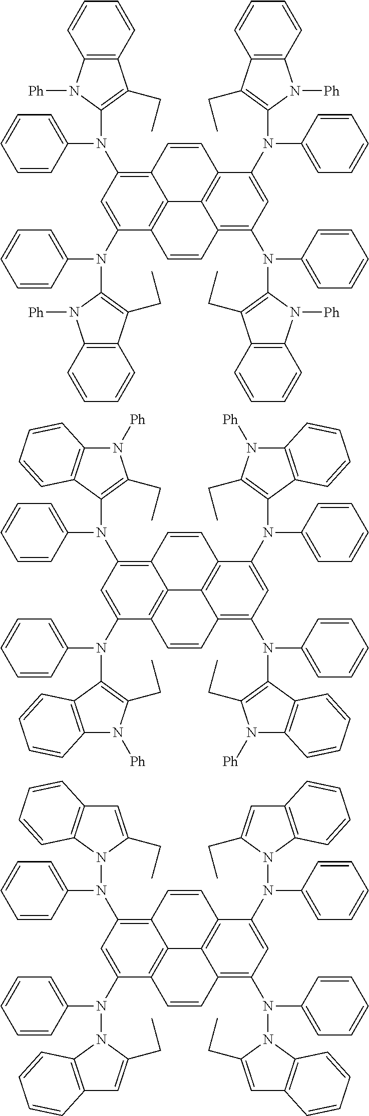

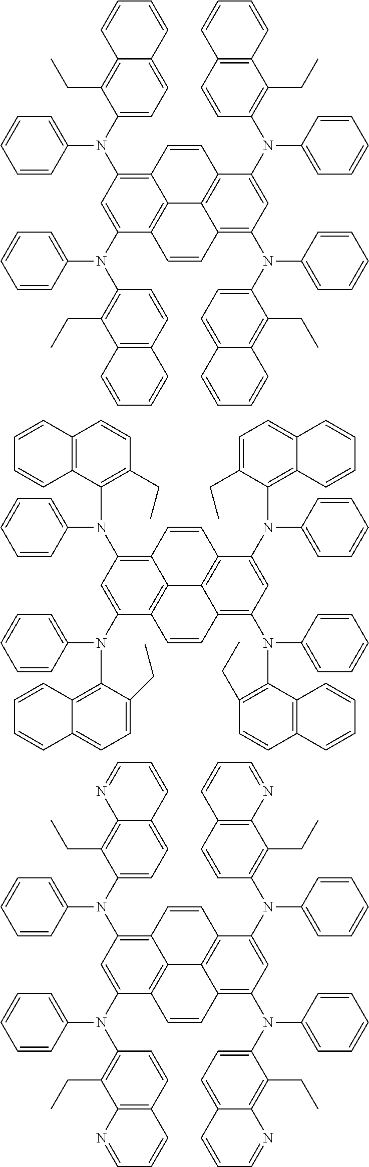

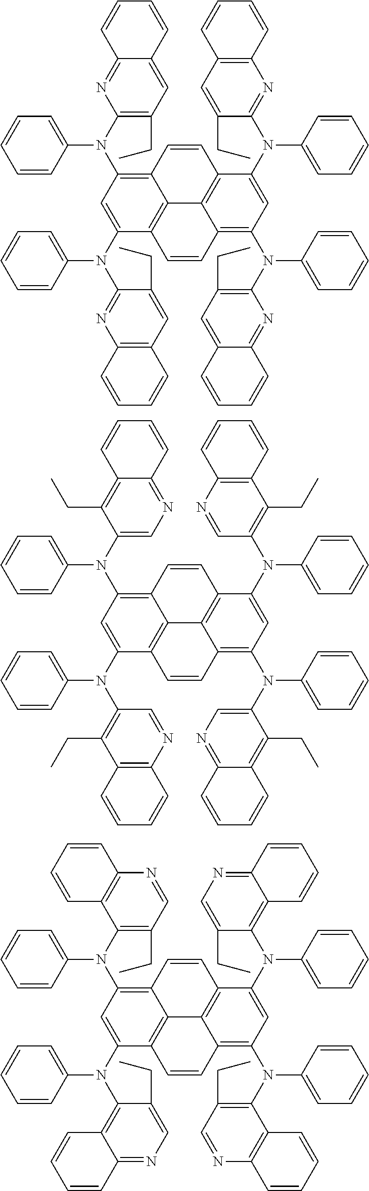

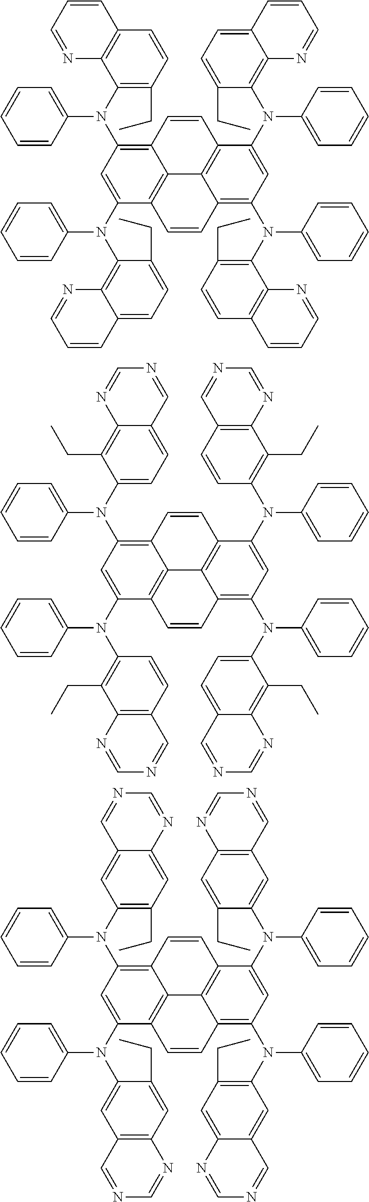

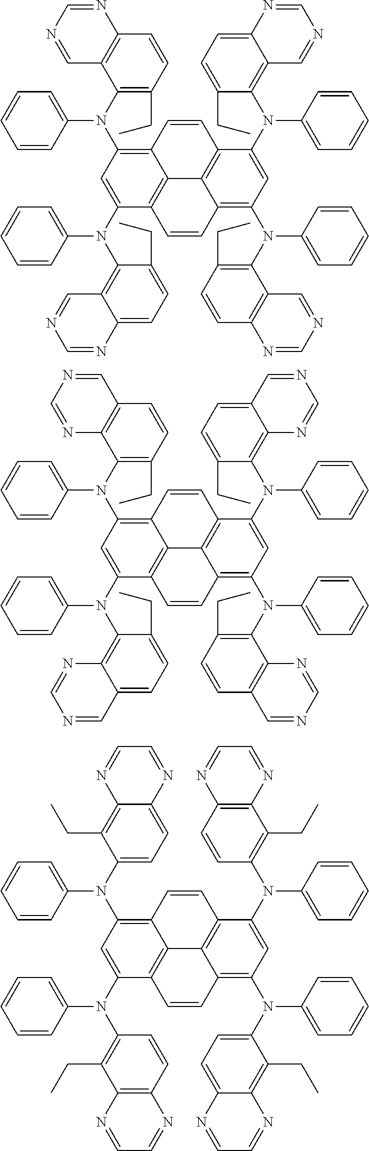

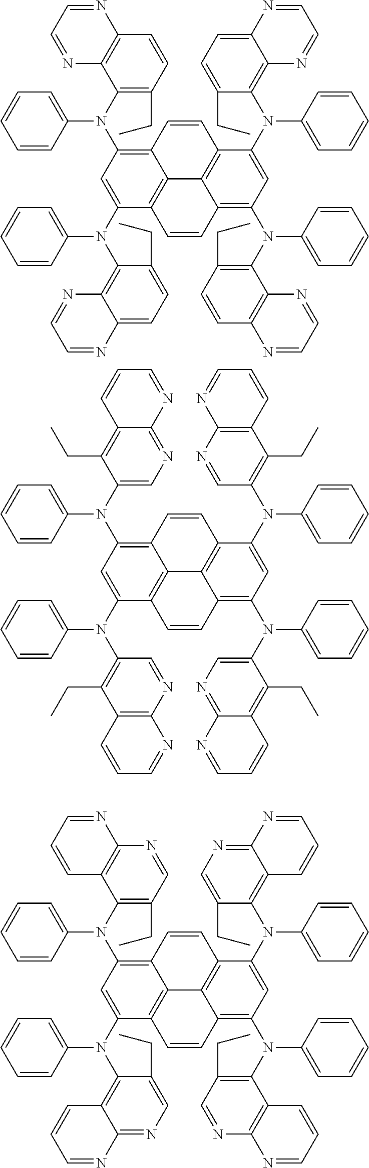

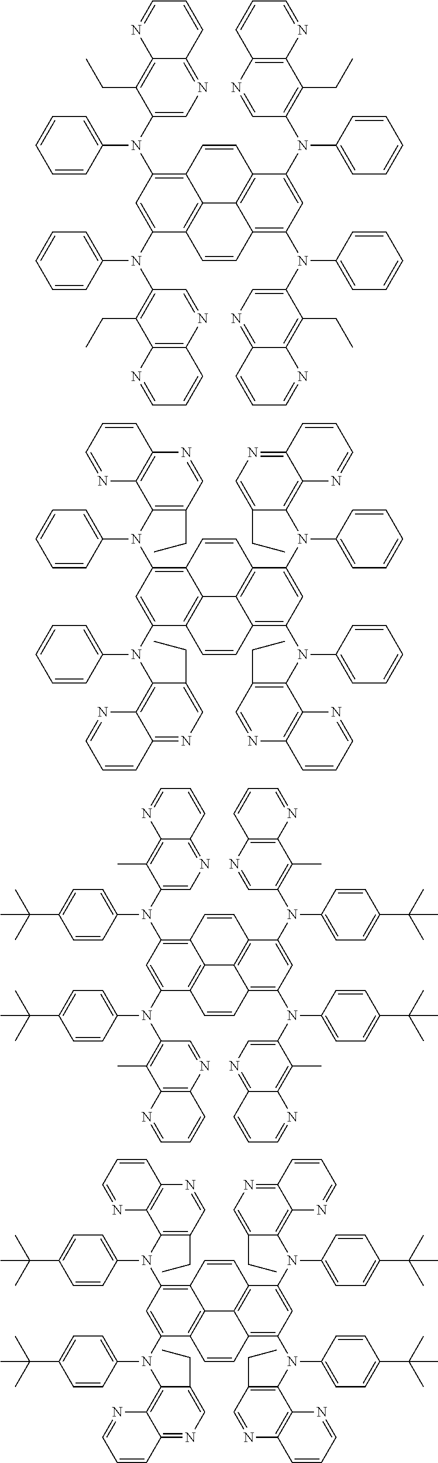

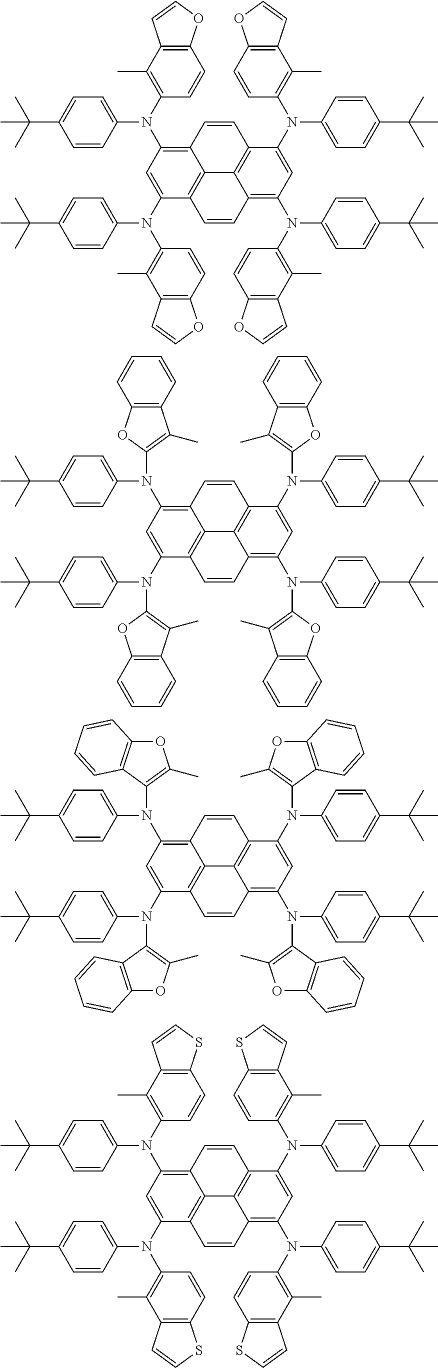

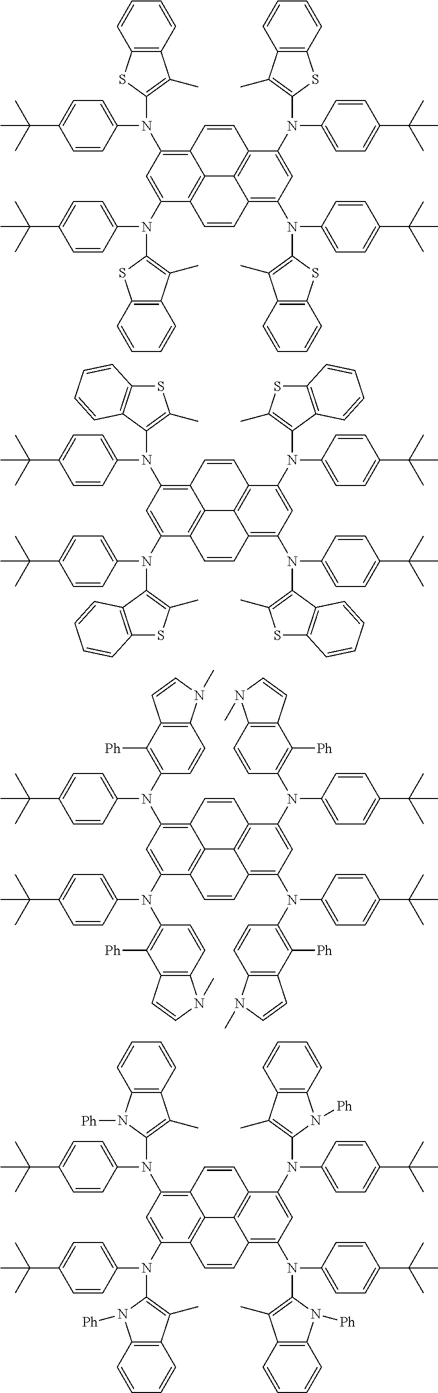

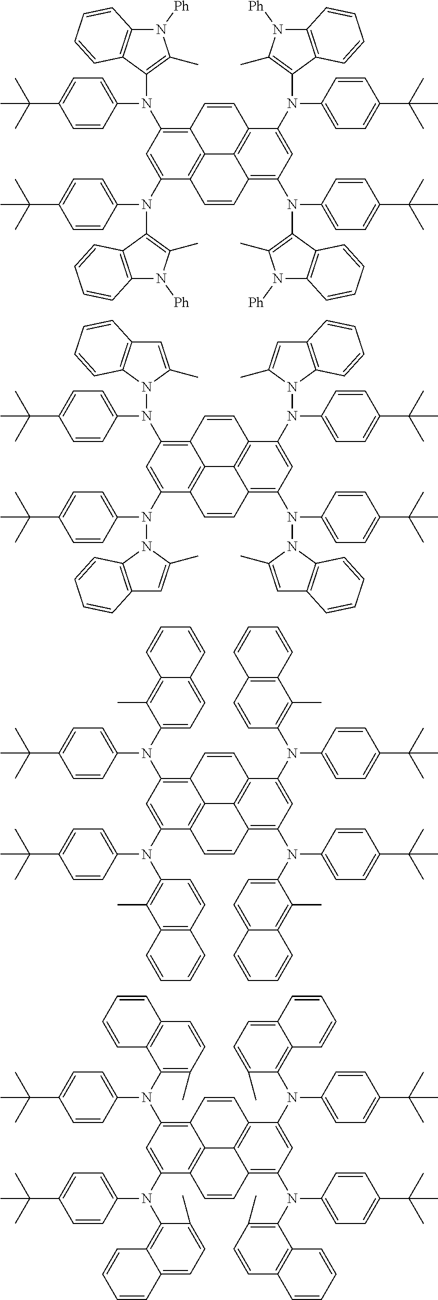

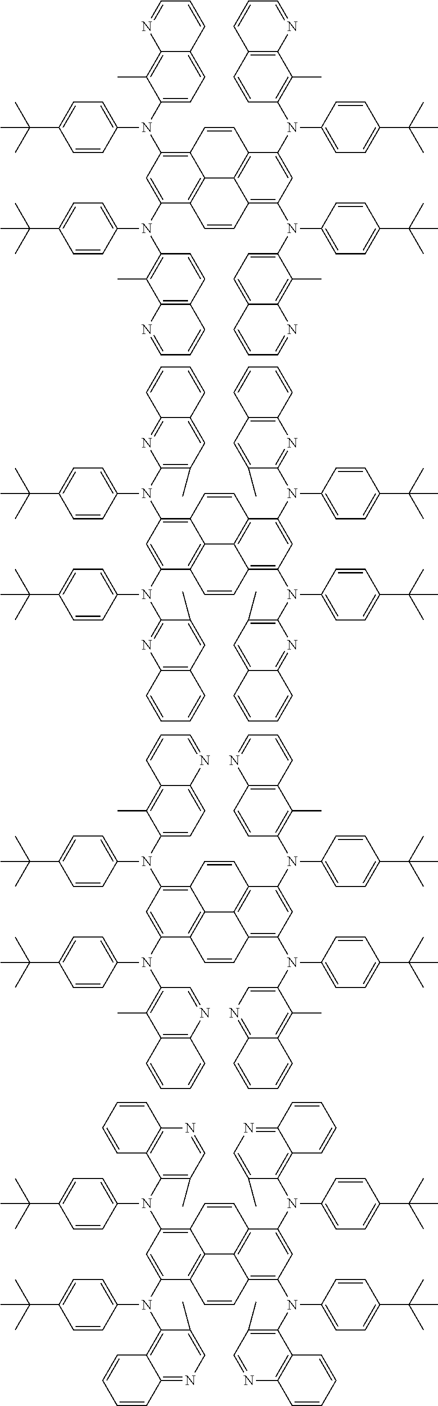

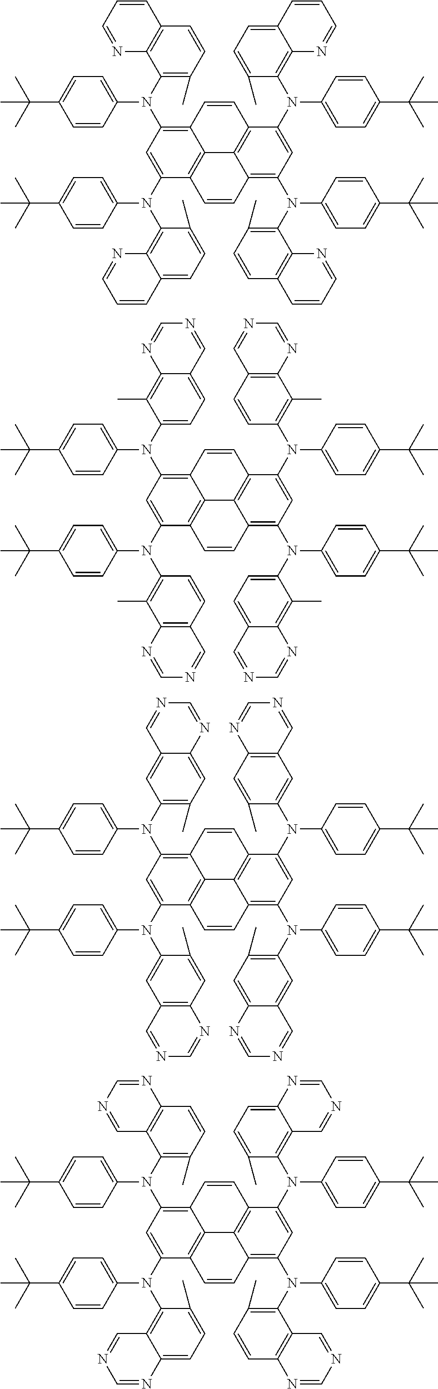

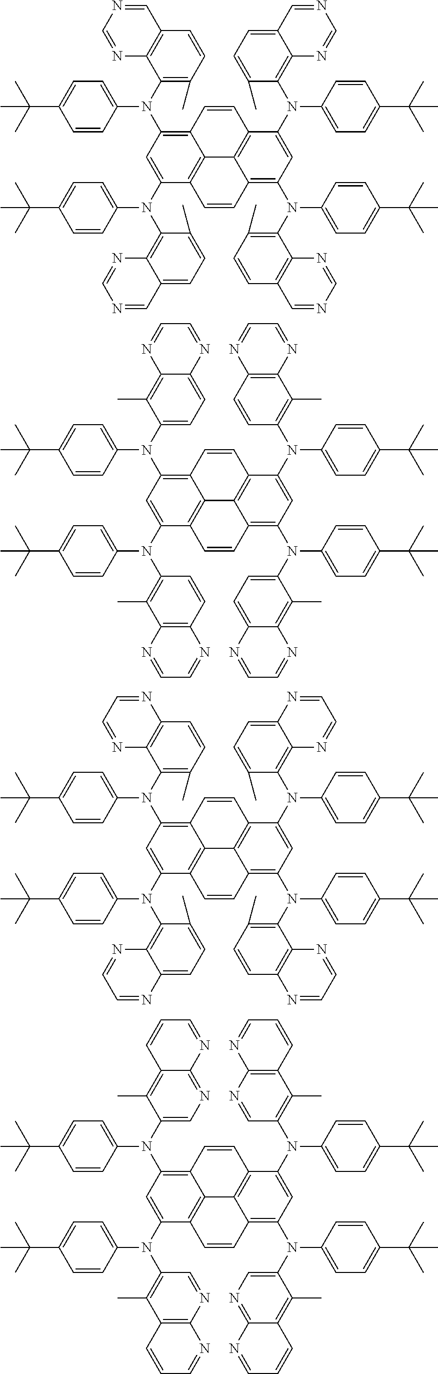

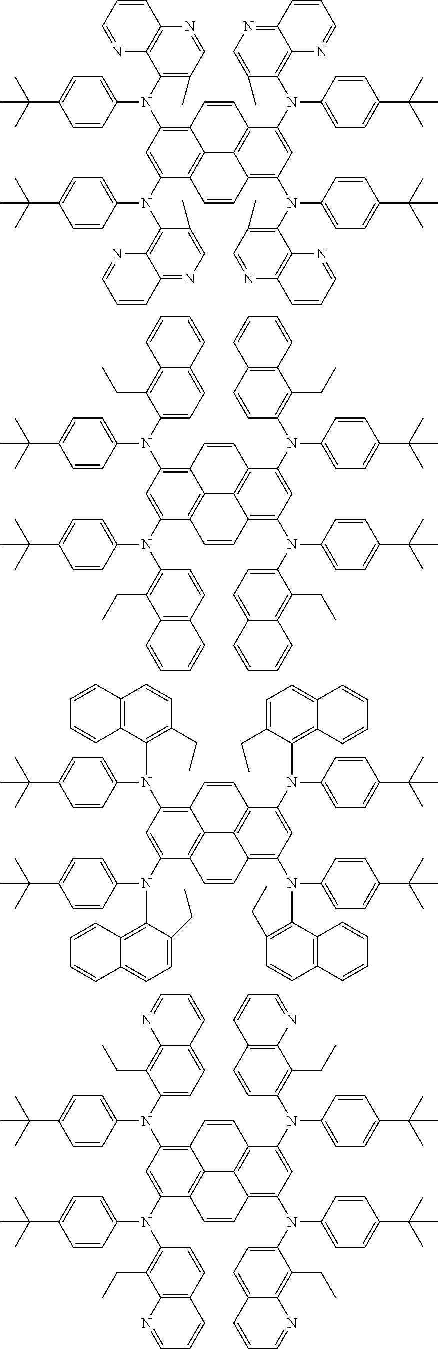

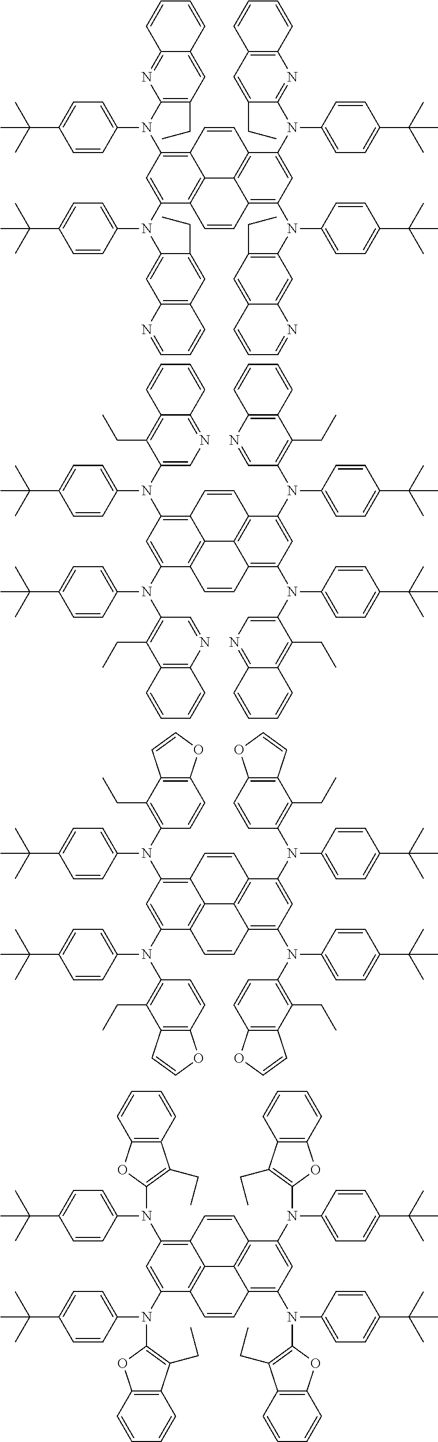

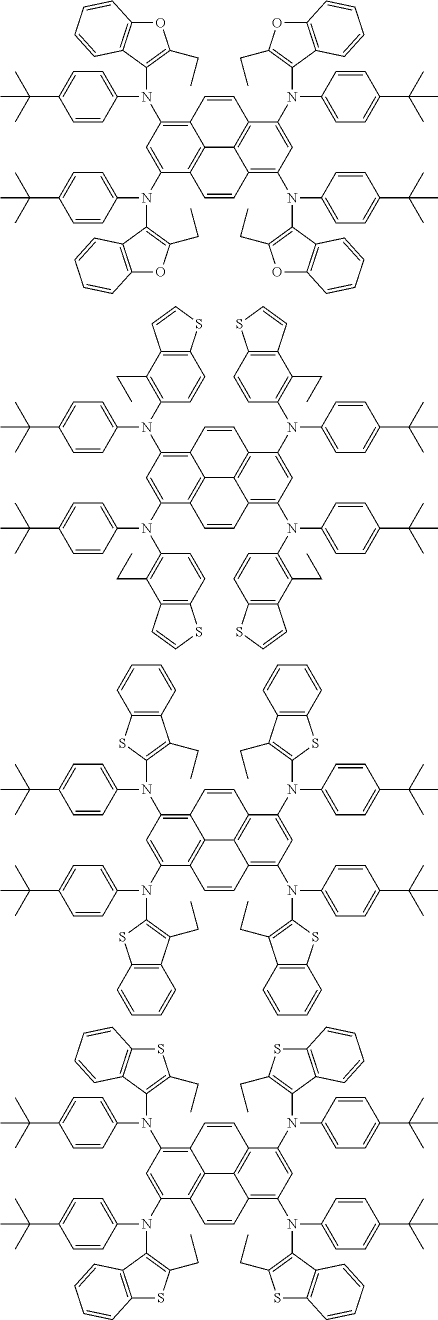

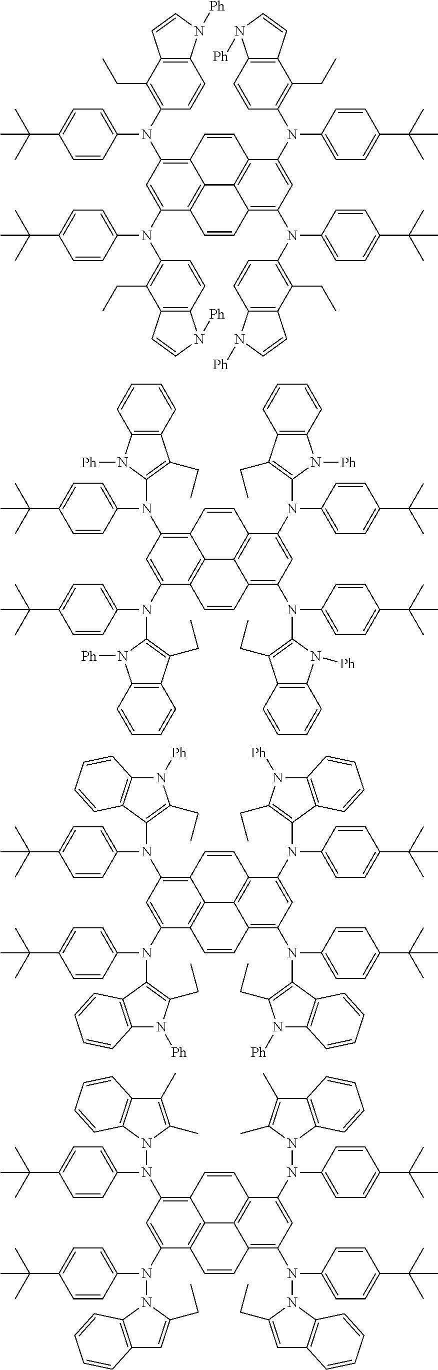

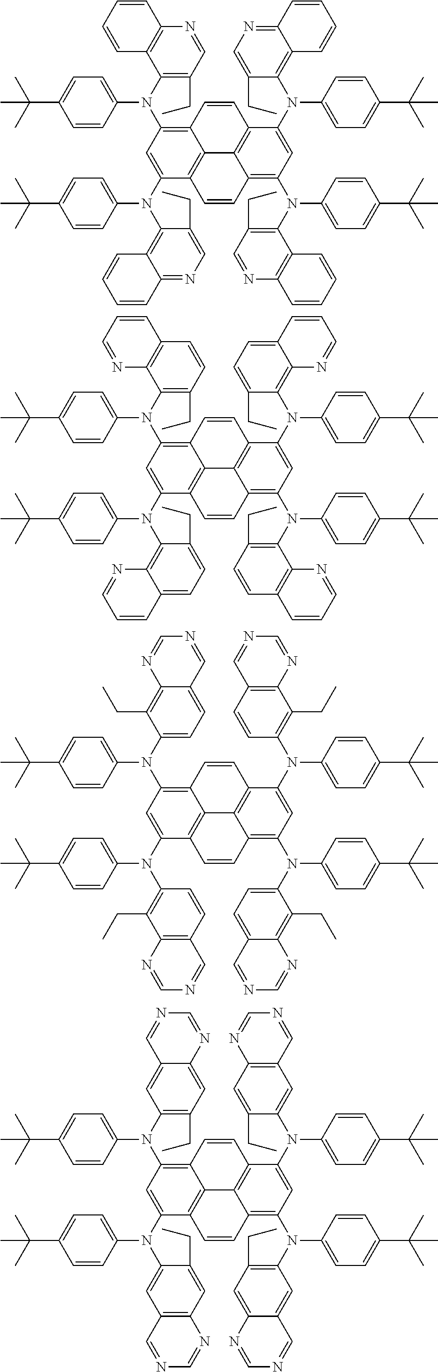

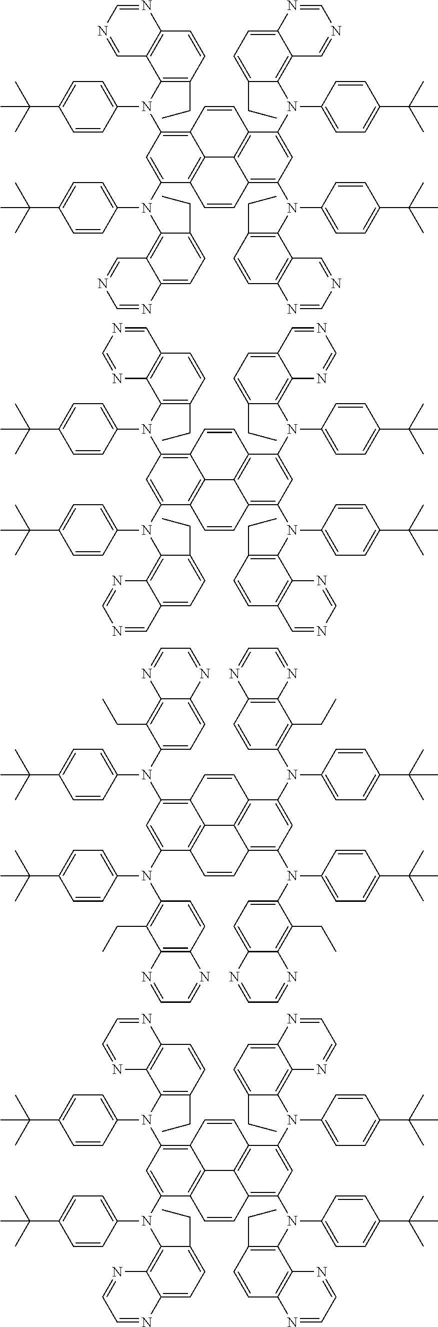

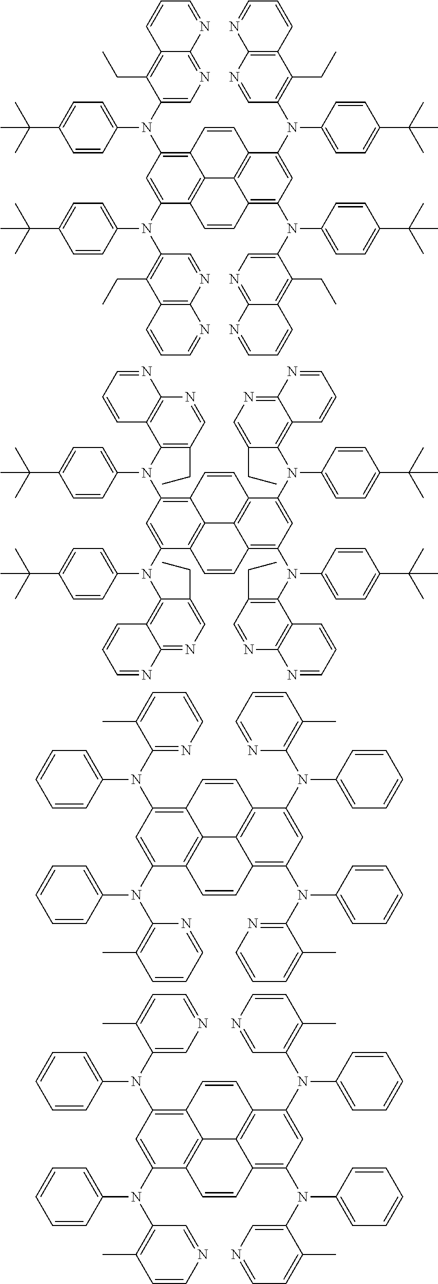

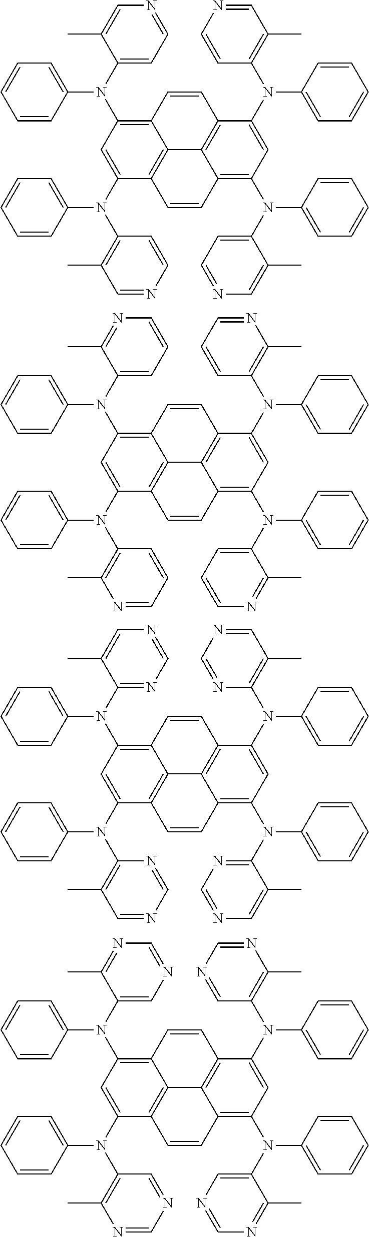

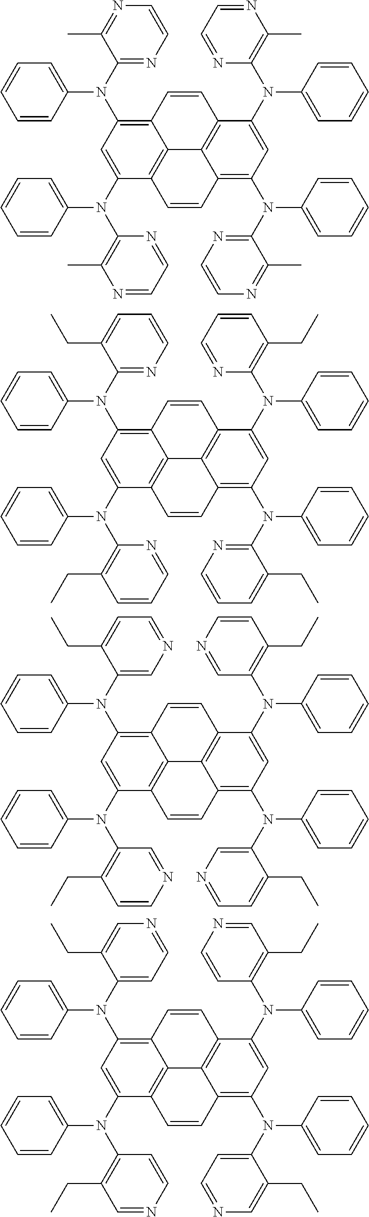

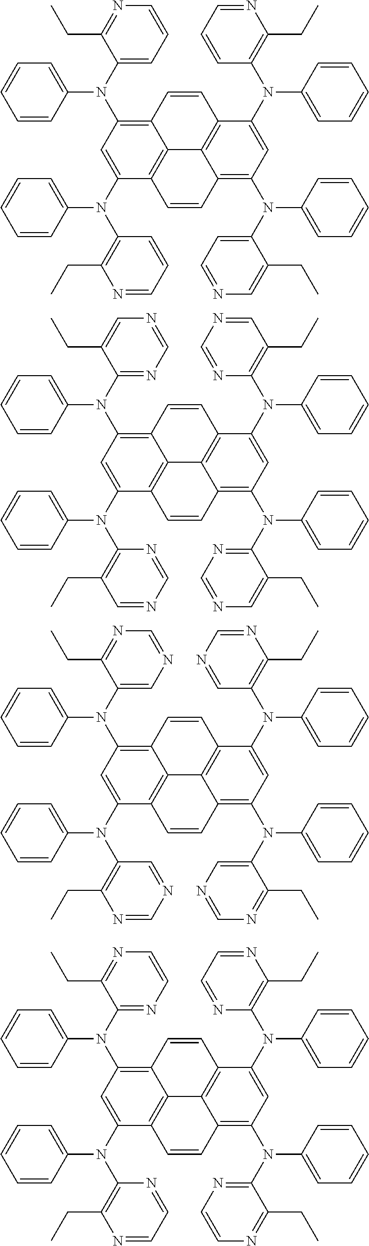

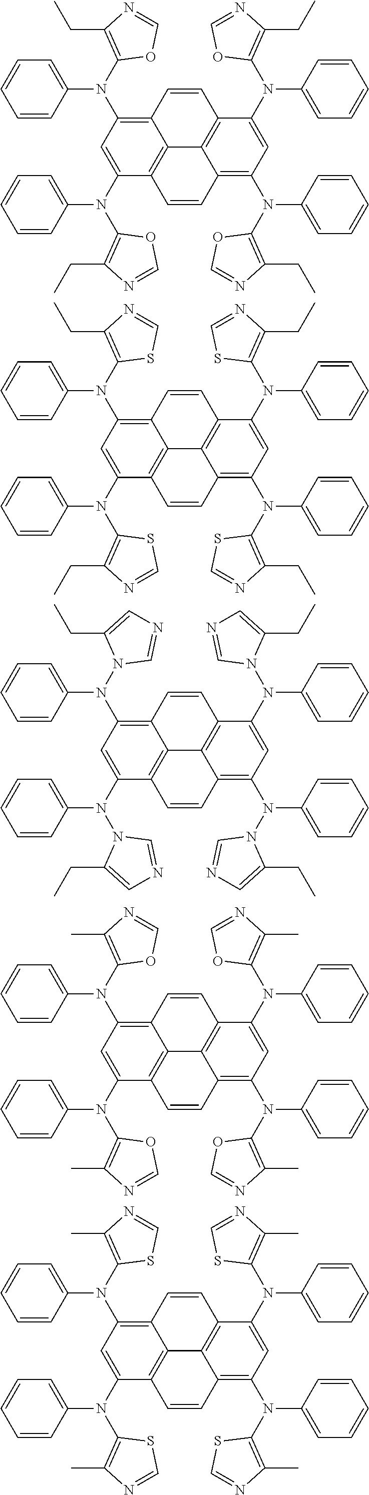

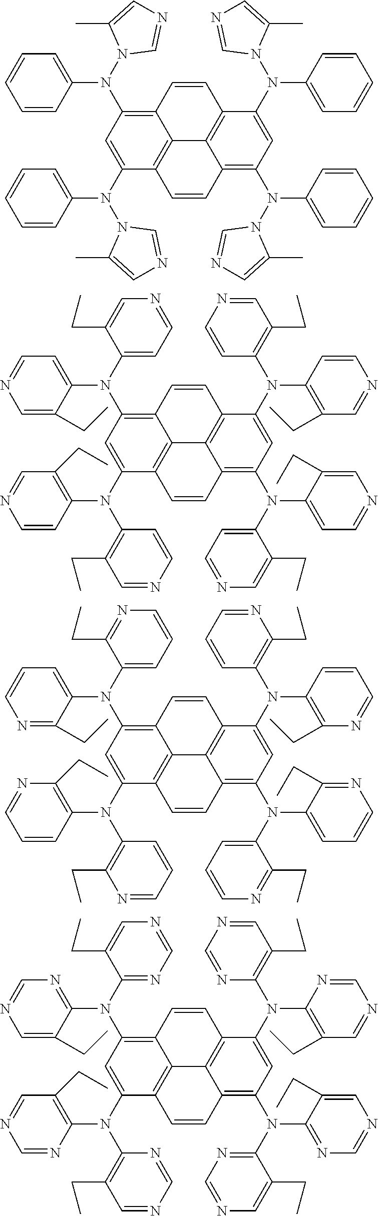

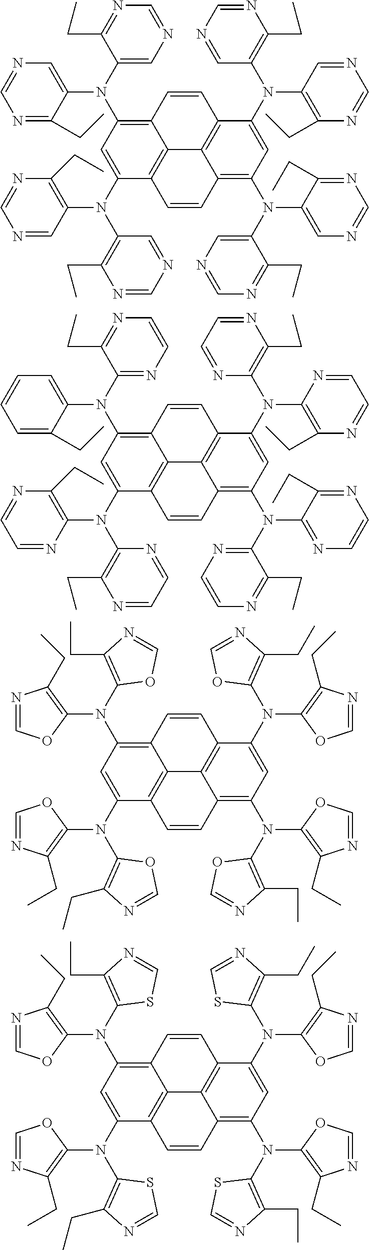

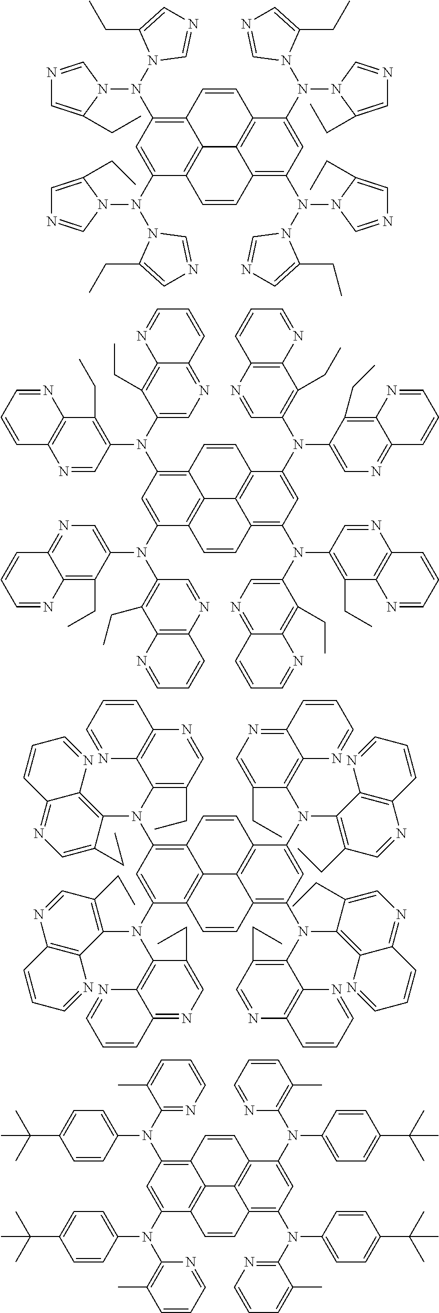

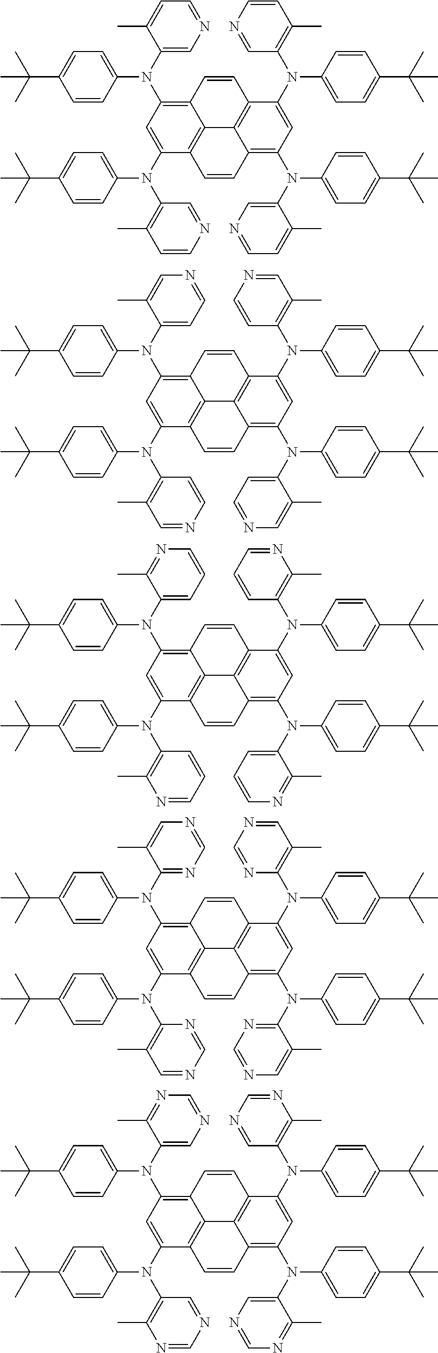

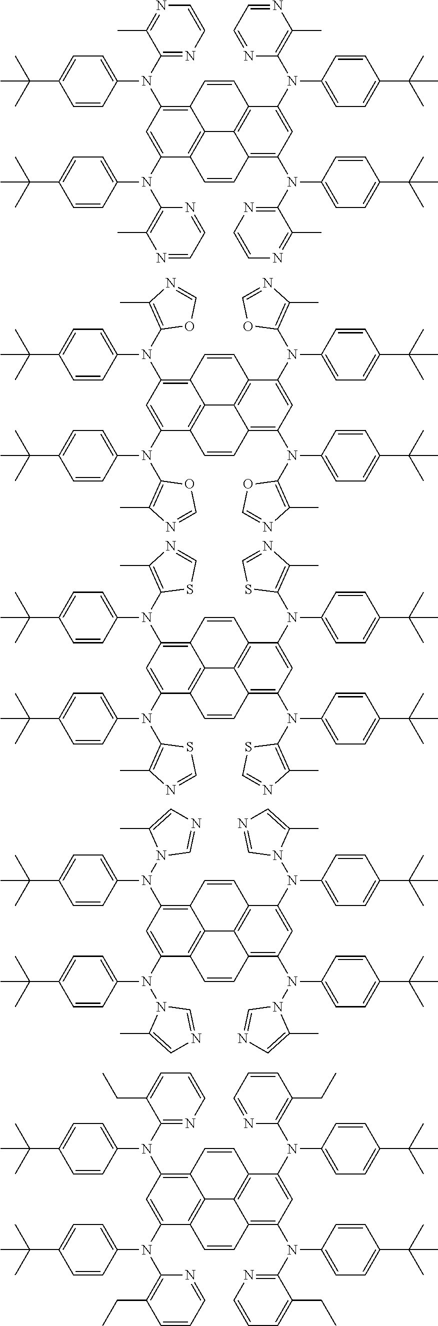

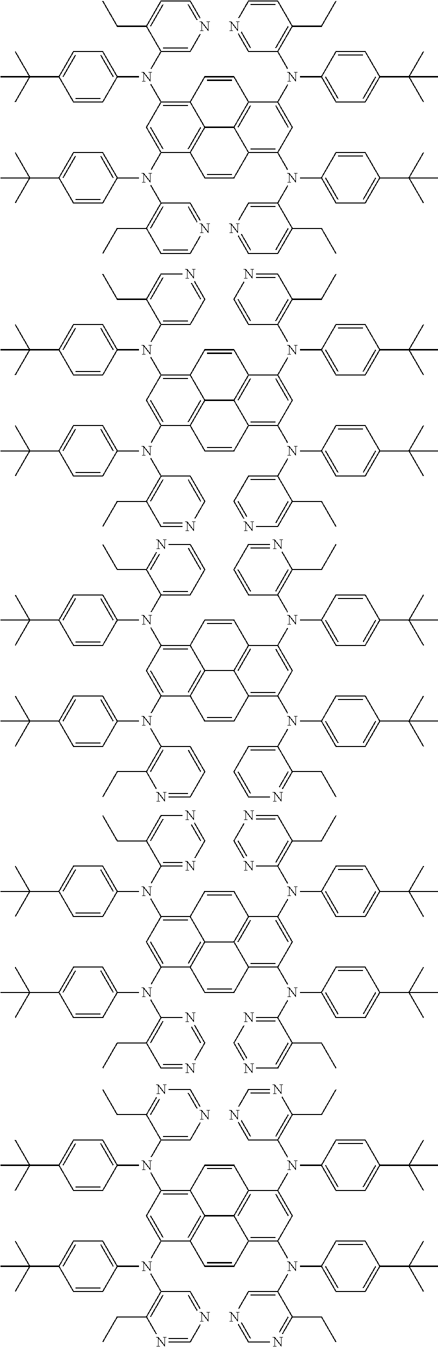

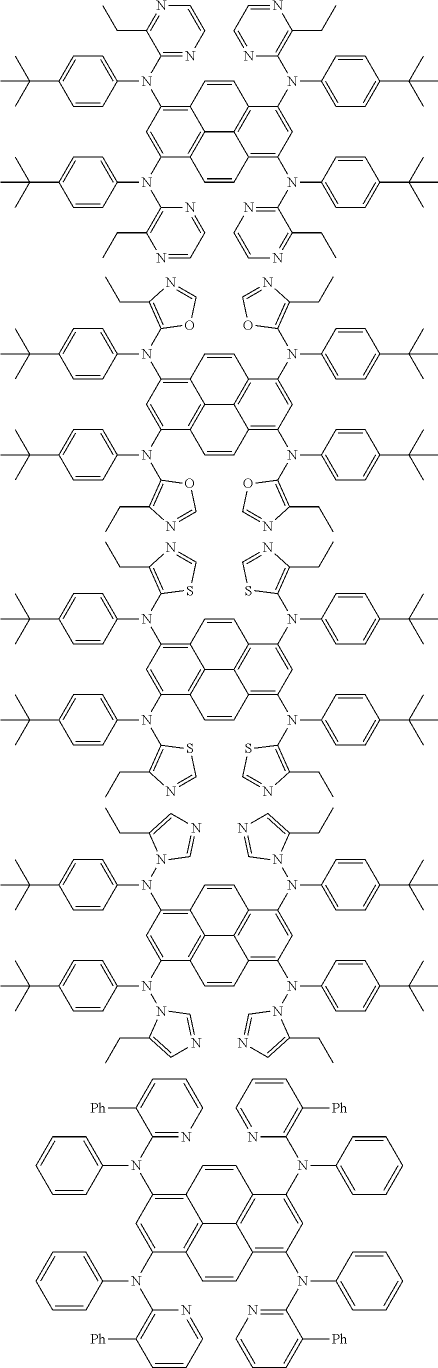

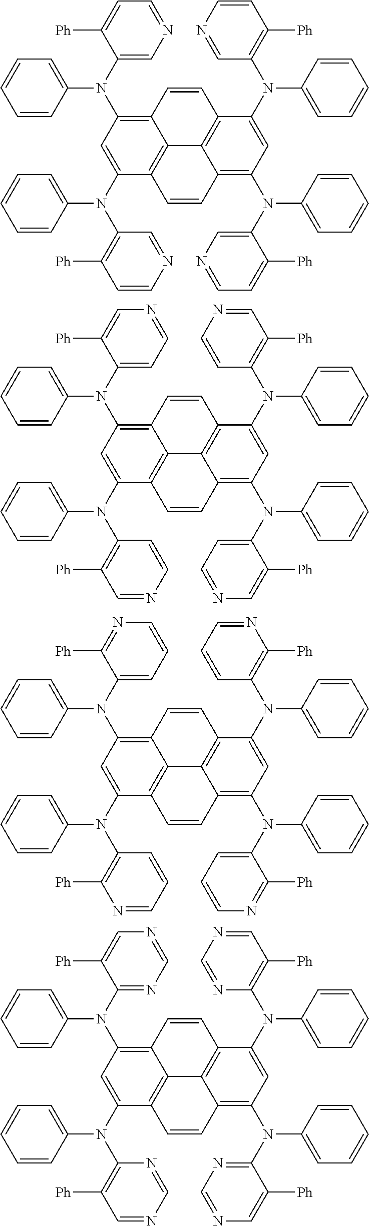

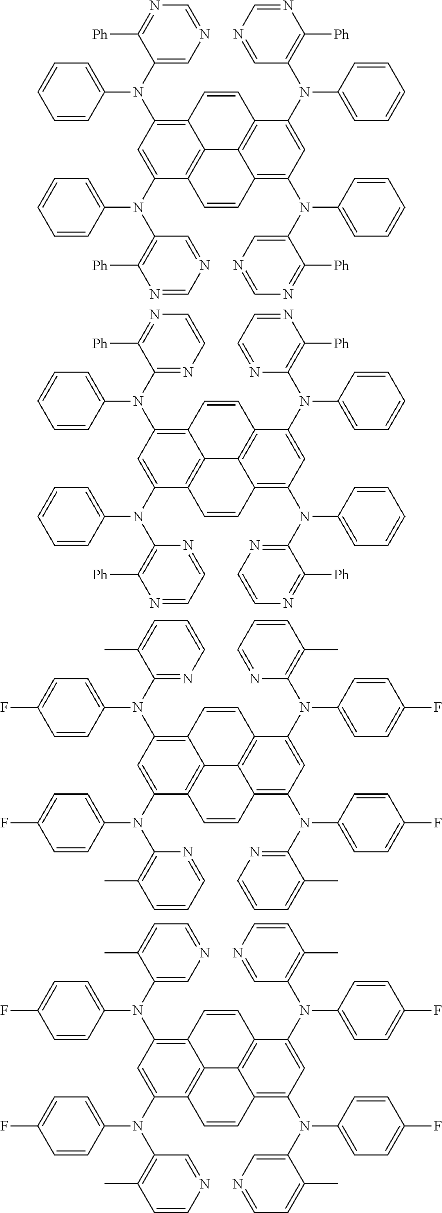

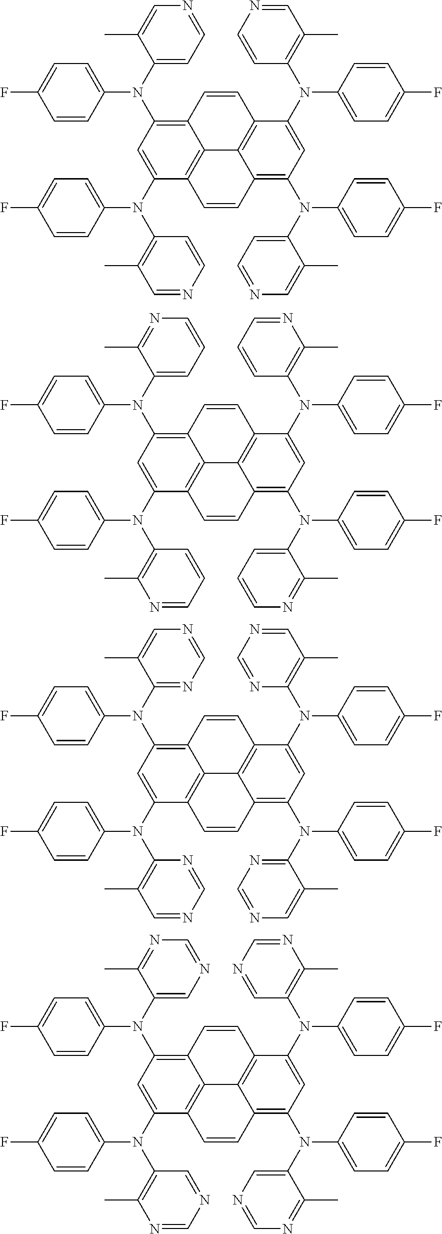

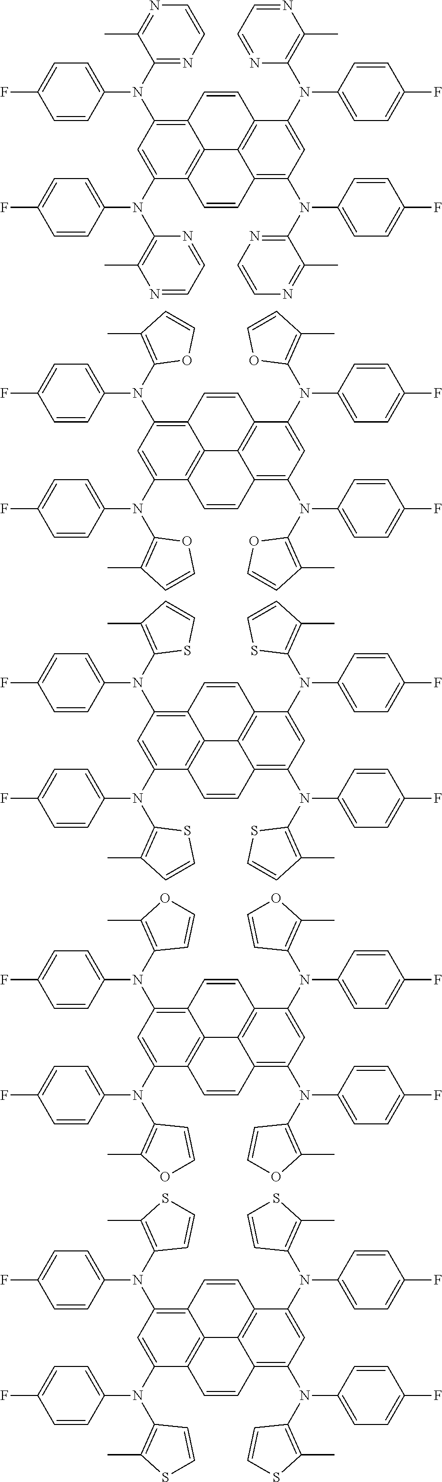

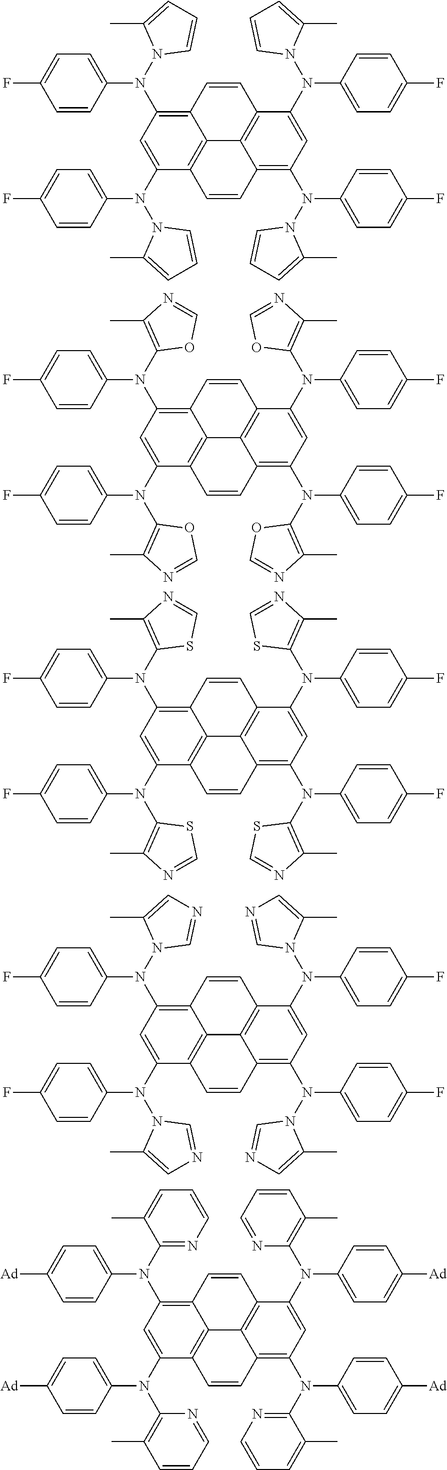

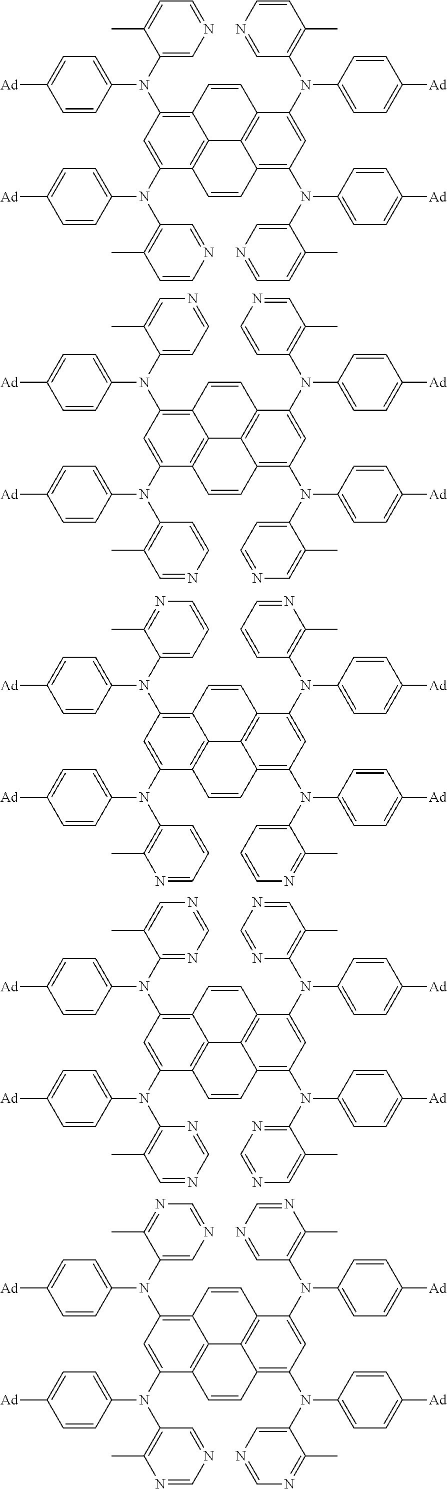

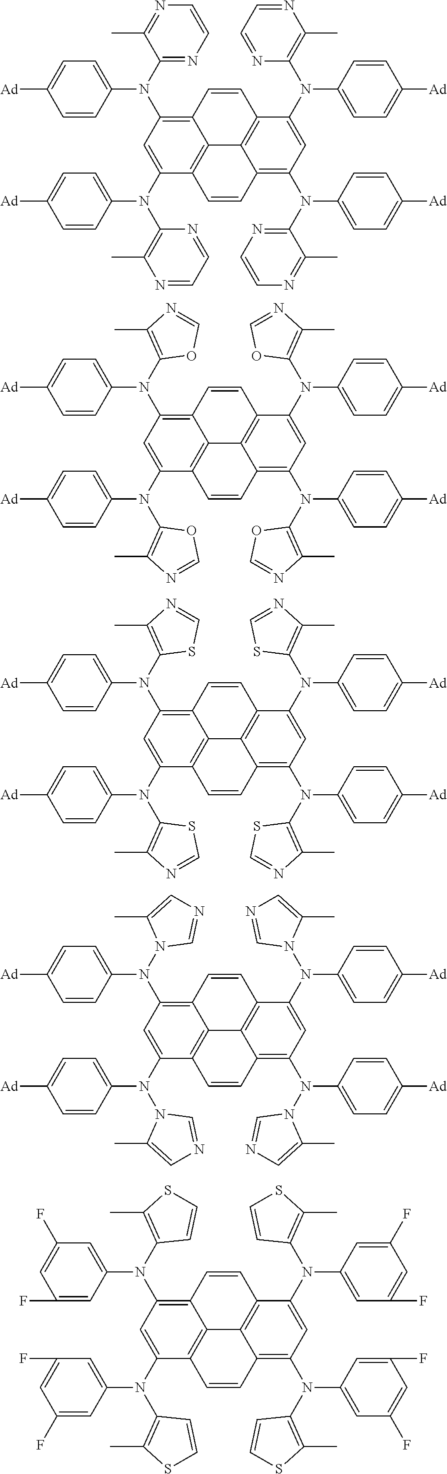

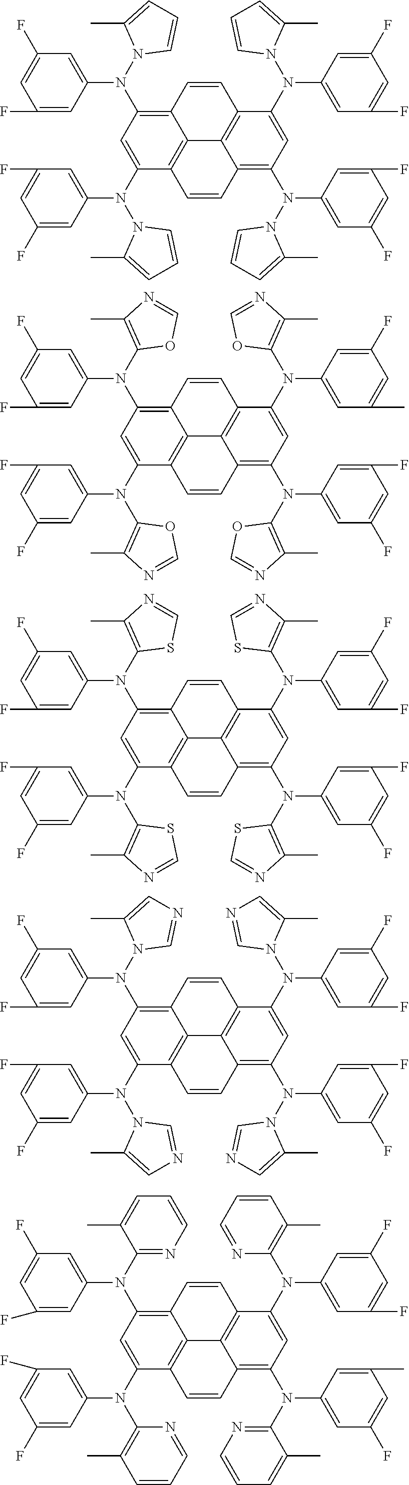

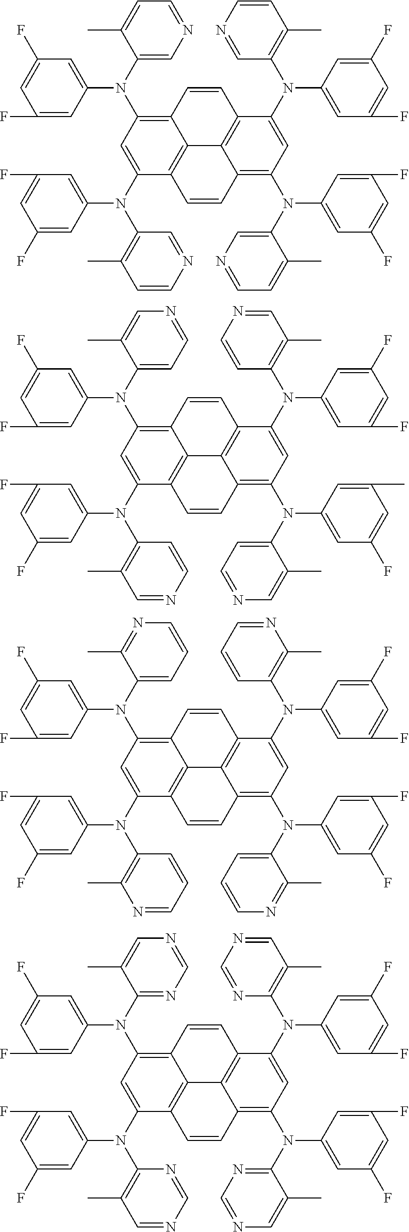

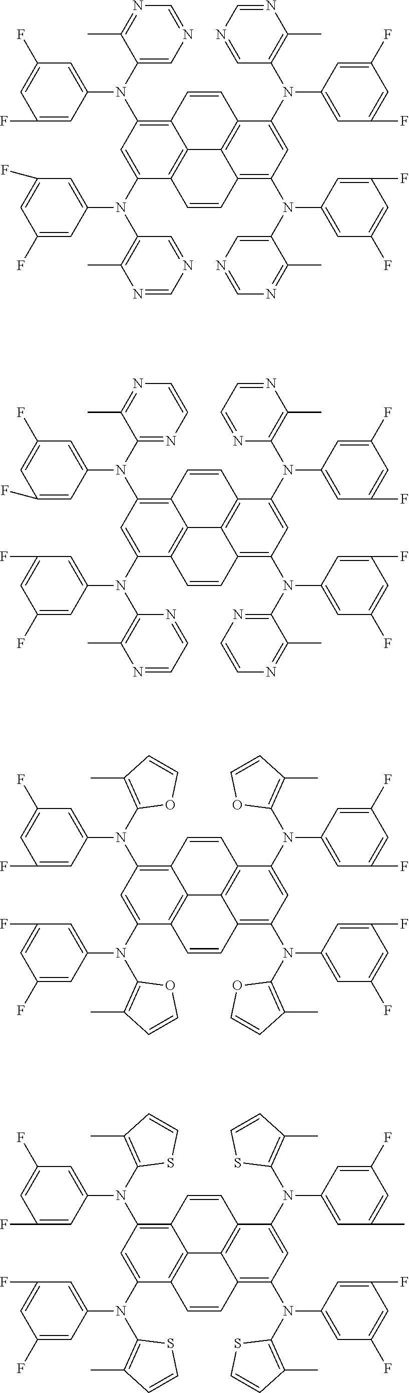

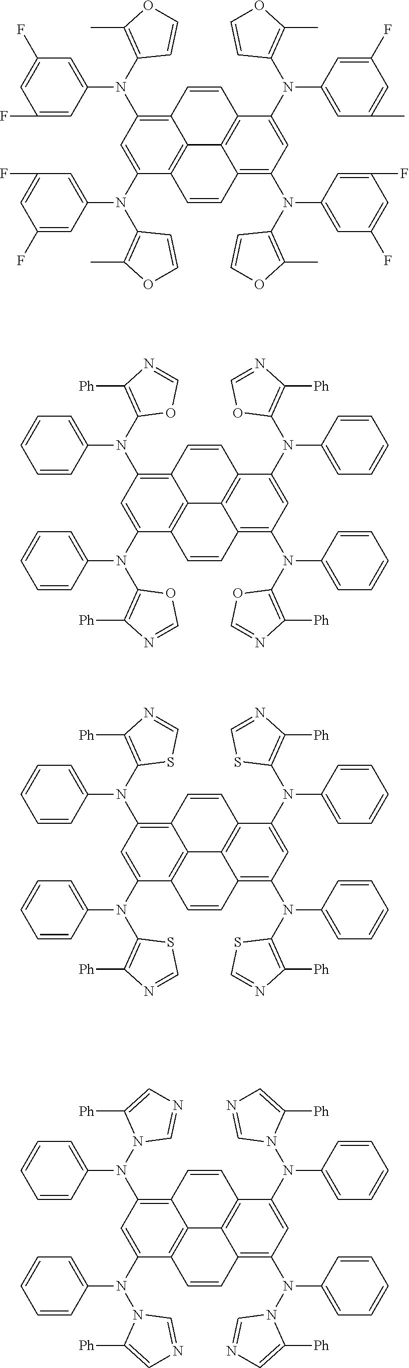

In another aspect, the present disclosure also provides a polymer comprising at least one repeating unit, where the at least one repeating unit comprises a structure corresponding to a compound as described herein.

Preferably, the polymer is a side chain polymer comprising a repeating unit of formula (II), where the repeating unit U comprises a structure corresponding to a compound as described herein, n₁ is an integer greater than or equal to 1.

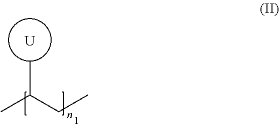

In some embodiments, the content of the repeating unit U in the polymer is from 0.1 mol % to 100 mol %.

In some embodiments, the content of the repeating unit U in the polymer is from 1 mol % to 90 mol %, preferably from 10 mol % to 90 mol %, more preferably from 20 mol % to 80 mol %, further preferably from 30 mol % to 70 mol %, and most preferably from 40 mol % to 60 mol %.

In yet another aspect, the present disclosure further provides a mixture comprising at least one compound or polymer as described herein, and another functional material. The another functional material is an organic functional material, which can be selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye. These organic functional materials are described in detail, for example, in WO2010135519A1, US20090134784A1, and WO2011110277A1. The entire contents of these three documents are hereby incorporated into this document for reference.

In some embodiments, the mixture comprises a compound or polymer as described herein, and a light-emitting material. Herein the compound or the polymer as described herein can be used as a host material, and the weight percentage of the light-emitting material ≤15 wt %, preferably ≤12 wt %, more preferably ≤9 wt %, further preferably ≤8 wt %, and most preferably ≤7 wt %.

In some embodiments, the light-emitting material is an organic fluorescent emitter.

The fluorescent emitter (also referred to as singlet emitter) is described in detail below.

The singlet emitter tends to have a long conjugated x-electron system. Hitherto, there have been many examples of styryl amines and derivatives thereof as disclosed in JP2913116B and WO2001021729A1, and indenofluorenes and derivatives thereof as disclosed in WO2008006449 and WO2007140847.

In some embodiments, the singlet emitter can be selected from the group consisting of monostyrylamines, distyrylamines, tristyrylamines, tetrastyrylamines, styrenphosphines, styrenethers, and arylamines.

A monostyrylamine refers to a compound which comprises one unsubstituted or substituted styryl group and at least one amine, most preferably an aryl amine. Distyrylamine refers to a compound comprising two unsubstituted or substituted styryl groups and at least one amine, most preferably an aryl amine. Ternarystyrylamine refers to a compound which comprises three unsubstituted or substituted styryl groups and at least one amine, most preferably an aryl amine. Quaternarystyrylamine refers to a compound comprising four unsubstituted or substituted styryl groups and at least one amine, most preferably an aryl amine. Preferred styrene is stilbene, which may be further substituted. The corresponding phosphines and ethers are defined similarly as amines. Aryl amine or aromatic amine refers to a compound comprising three unsubstituted or substituted cyclic or heterocyclic aryl systems directly attached to nitrogen. At least one of these cyclic or heterocyclic aryl systems is preferably selected from fused ring systems and most preferably has at least 14 aryl ring atoms. Among the preferred examples are aryl anthramine, aryl anthradiamine, aryl pyrene amines, aryl pyrene diamines, aryl chrysene amines and aryl chrysene diamine. Aryl anthramine refers to a compound in which one diarylamino group is directly attached to anthracene, most preferably at position 9. Aryl anthradiamine refers to a compound in which two diarylamino groups are directly attached to anthracene, most preferably at positions 9,10. Aryl pyrene amines, aryl pyrene diamines, aryl chrysene amines and aryl chrysene diamine are similarly defined, where the diarylarylamino group is most preferably attached to position 1 or 1,6 of pyrene.

Examples of singlet emitters based on vinylamines and arylamines, which are also preferred, may be found in the following patent publications: WO2006000389, WO2007065549, DE102005058557A1, CN1583691A, U.S. Pat. No. 6,251,531B1, US2006210830A, and US20080113101A1. The patent publications listed above are specially incorporated herein by reference in their entirety.

Examples of singlet emitters based on stilbene and its derivatives may be found in U.S. Pat. No. 5,121,029.

Further preferred singlet emitter can be selected from the group consisting of indenofluorene-amine and indenofluorene-diamine, as disclosed in WO2006122630, benzoindenofluorene-amine and benzoindenofluorene-diamine, as disclosed in WO2008006449, dibenzoindenofluorene-amine and dibenzoindenofluorene-diamine, as disclosed in WO2007140847.

Other materials that can be used as singlet emitters include polycyclic aromatic hydrocarbon compounds, in particular selected from the derivatives of the following compounds: anthracene such as 9,10-di(2-naphthyl) anthracene, naphthalene, tetraphenyl, phenanthrene, perylene such as 2,5,8,11-tetra-t-butylatedylene, indenoperylene, phenylene (benzo fused ring such as 4,4′-(bis(9-ethyl-3-carbazovinylene)-1,1′-biphenyl)), periflanthene, decacyclene, coronene, fluorene, spirobifluorene, arylpyren, arylenevinylene, cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds, bis (azinyl) methene compounds, carbostyryl compounds, oxazone, benzoxazole, benzothiazole, benzimidazole, or diketopyrrolopyrrole. Some singlet emitter materials may be found in the following patent publications: US20070252517A1, U.S. Pat. Nos. 4,769,292, 6,020,078. The patent publications listed above are specially incorporated herein by reference in their entirety.

The following are some examples of singlet emitters:

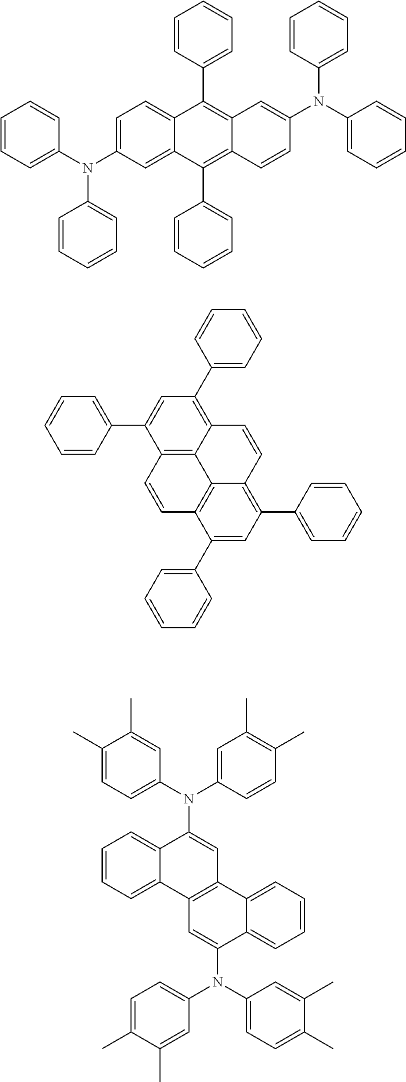

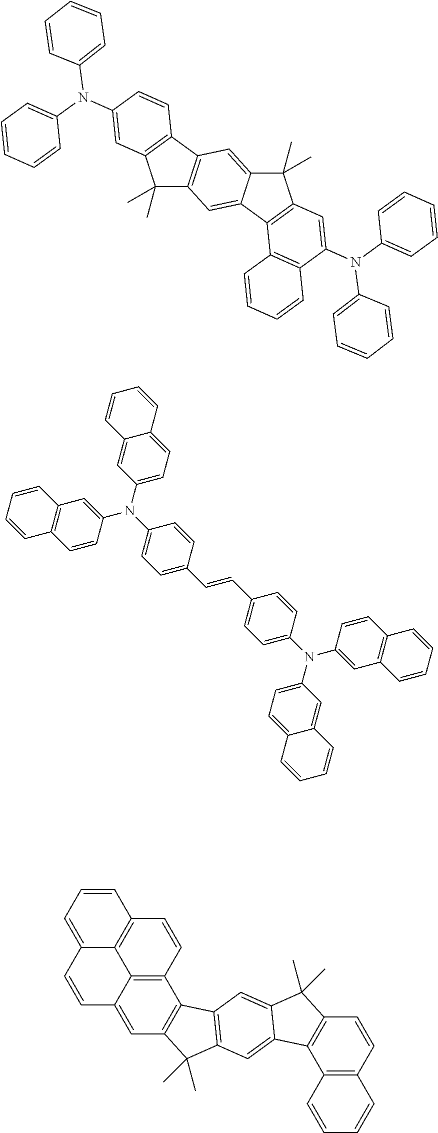

In some embodiments, the mixture comprises a compound or a polymer (as a host material H) as described herein, and an emitter E, where 1) the emission spectrum of the compound or the polymer (i.e., the host material H) is on the short wavelength side of the absorption spectrum of the emitter E, and at least partially overlaps with the absorption spectrum of the emitter E; 2) the FWHM of the emission spectrum of the emitter E≤55 nm.

In some embodiments, the FWHM of the emission spectrum of the emitter E≤50 nm, preferably ≤40 nm, more preferably ≤35 nm, and most preferably ≤30 nm.

In some embodiments, the photoluminescence quantum yield (PLQY) of the emitter E ≥60%, preferably ≥65%, more preferably ≥70%, and most preferably ≥80%.

In some embodiments, the emitter E is an organic emitter comprising a structure of formula (1) or formula (2):

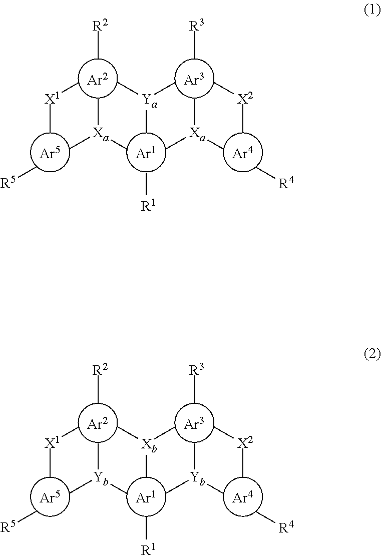

Where each of Ar¹ to Ar³ is independently selected from an aromatic group or a heteroaromatic group containing 5 to 24 ring atoms; each of Ar⁴ and Ar⁵ is independently selected from null, an aromatic group or a heteroaromatic group containing 5 to 24 ring atoms; when neither Ar⁴ nor Ar⁵ is null, each of Xₐ and X_{b} at each occurrence is independently selected from N, C(R⁶), or Si(R⁶); each of Yₐ and Y_{b} at each occurrence is independently selected from B, P═O, C(R⁶), or Si(R⁶); when Ar⁴ and/or Ar⁵ is null, each X_{b} is selected from N, C(R⁶), or Si(R⁶); each Yₐ is selected from B, P═O, C(R⁶), or Si(R⁶); each of Xₐ and Y_{b} at each occurrence is independently selected from N(R⁶), C(R⁶R⁷), Si(R⁶R⁷), C═O, O, C═N(R⁶), C═C(R⁶R⁷), P(R⁶), P(═O)R⁶, S, S═O, or SO₂; each of X¹ and X² is independently selected from null or a bridging group;

each of R¹ to R⁷ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof, where one or more R¹-R⁷ may form a monocyclic or polycyclic aliphatic or aromatic ring system with each other and/or with the rings bonded thereto.

Examples of organic emitters according to formula (1) or formula (2) those are disclosed in WO Publication No. 2022078429 and WO Publication No. 2022213993, which are hereby incorporated by reference in its entirety.

In addition, for the purposes of the mixture as described herein, the emitter E may be further selected from an organic compound (i.e., Bodipy derivatives) having the following structural formula:

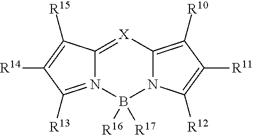

Where X is CR¹⁸ or N; each of R¹⁰ to R¹⁸ is independently selected from a hydrogen, an alkyl group, a cycloalkyl group, a heterocyclic group, an alkenyl group, a cycloalkenyl group, an alkynyl group, a hydroxyl group, a sulfhydryl group, an alkoxy group, an alkylthio group, an aryl ether group, an aryl thioether group, an aryl group, a heteroaryl group, a halogen, a cyano group, an aldehyde group, a carbonyl group, a carboxyl group, an oxycarboxyl group, a carbamoyl group, an amino group, a nitro group, a silyl group, a siloxanyl group, a boranyl group, or a phosphine oxide group; R¹⁰-R¹⁸ may form a fused ring and an aliphatic ring with the adjacent substituents.

In some embodiments, each of R¹⁶ and R¹⁷ is an electron-withdrawing group.

Suitable electron-withdrawing groups include, but not limited to, F, Cl, a cyano group, a partial/perfluorinated alkyl chain, or one of the following groups:

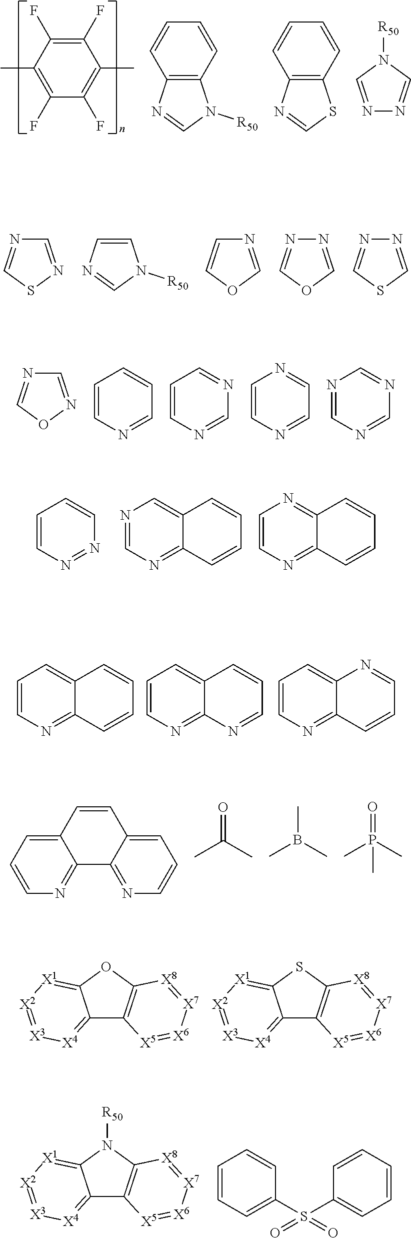

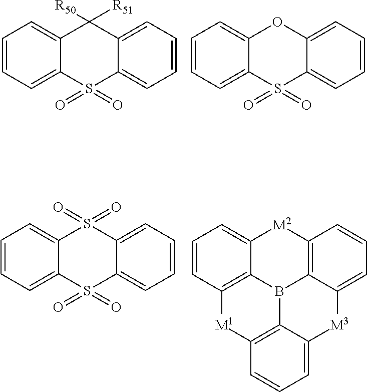

Where X¹-X⁸, M¹, M², M³, R₅₀, R₅₁, and n are identically defined as described herein.

Examples of suitable Bodipy derivatives include, but not limited to:

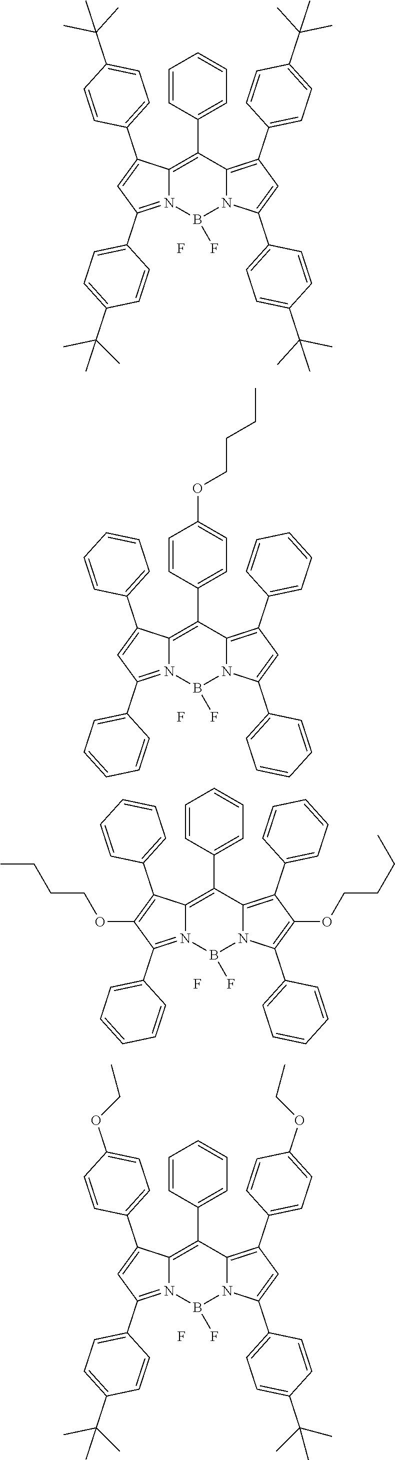

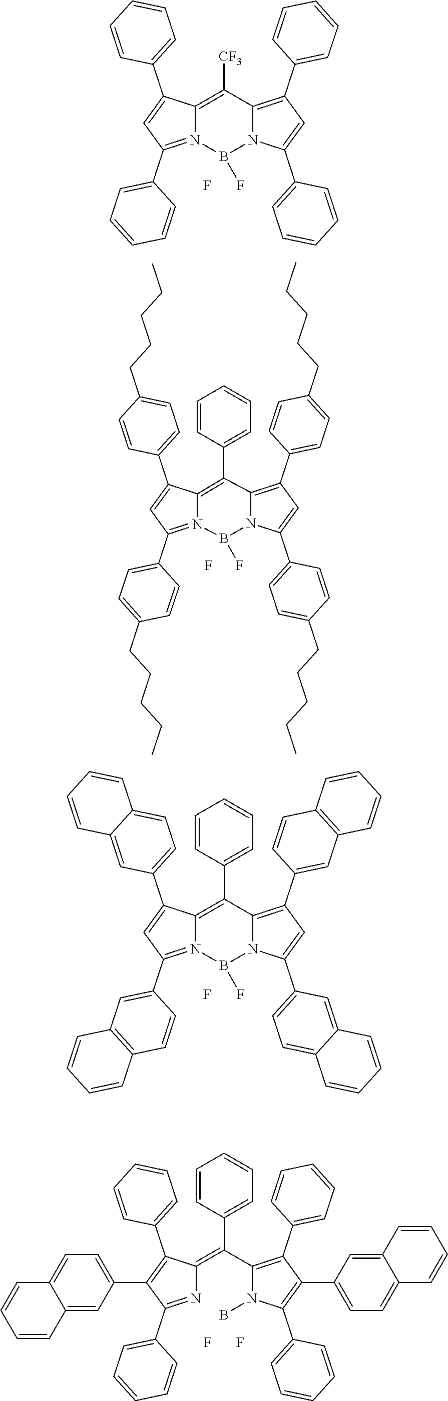

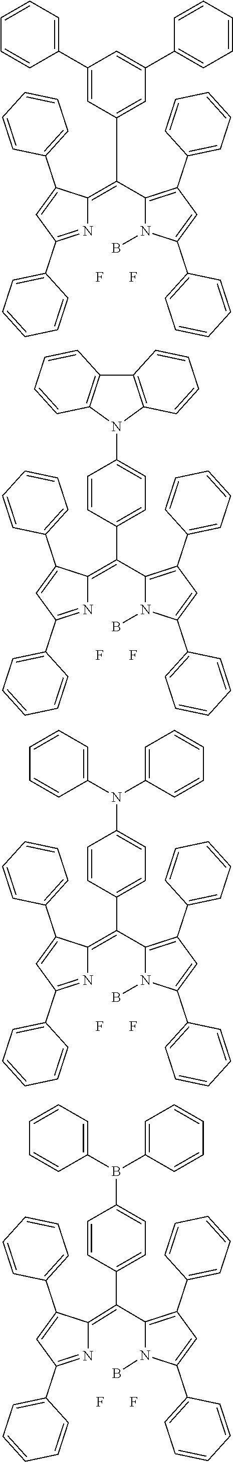

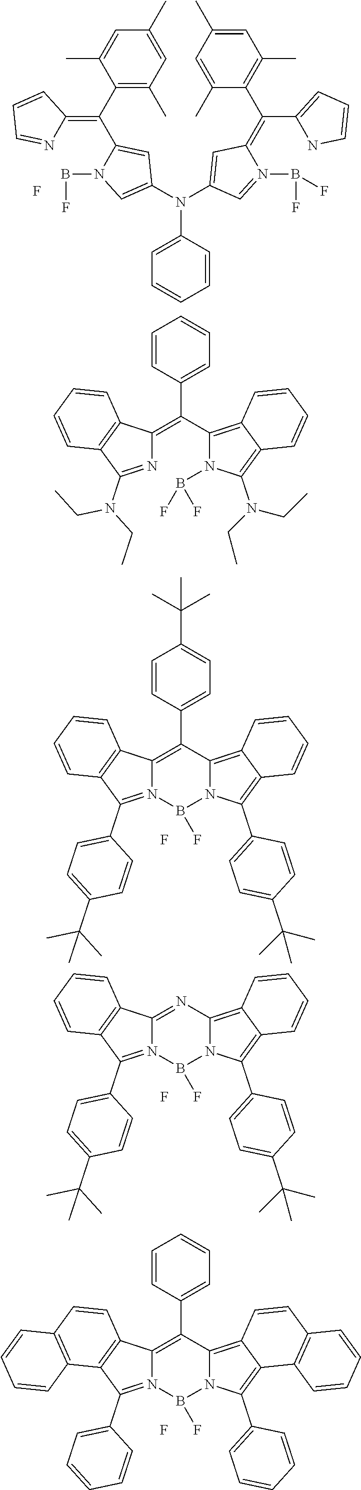

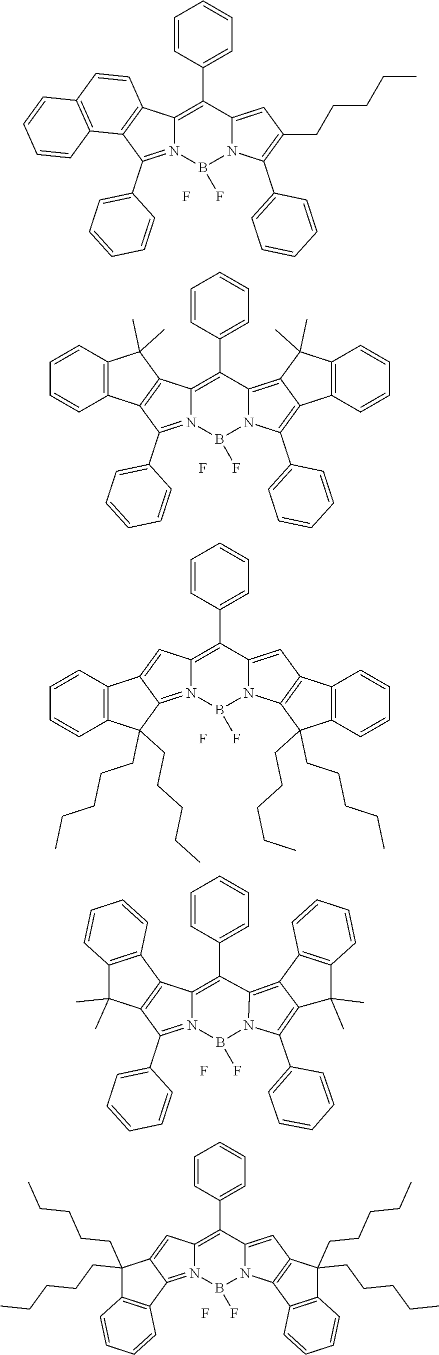

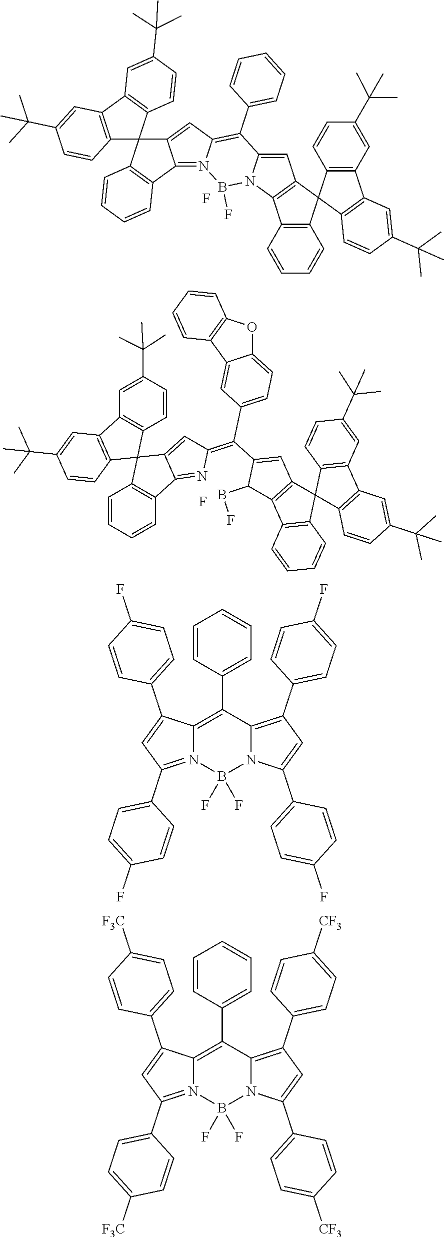

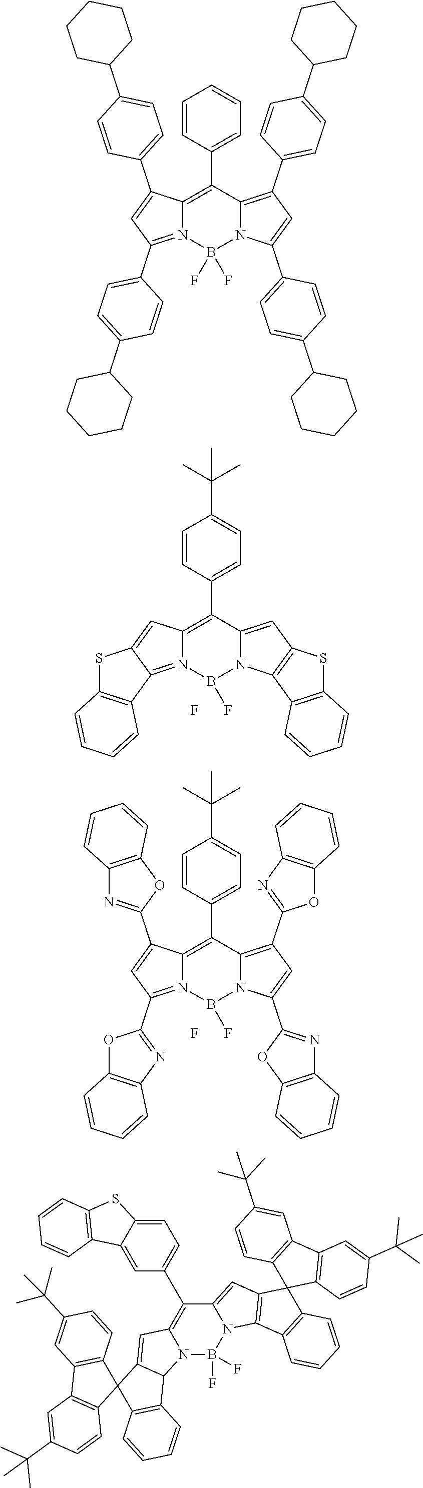

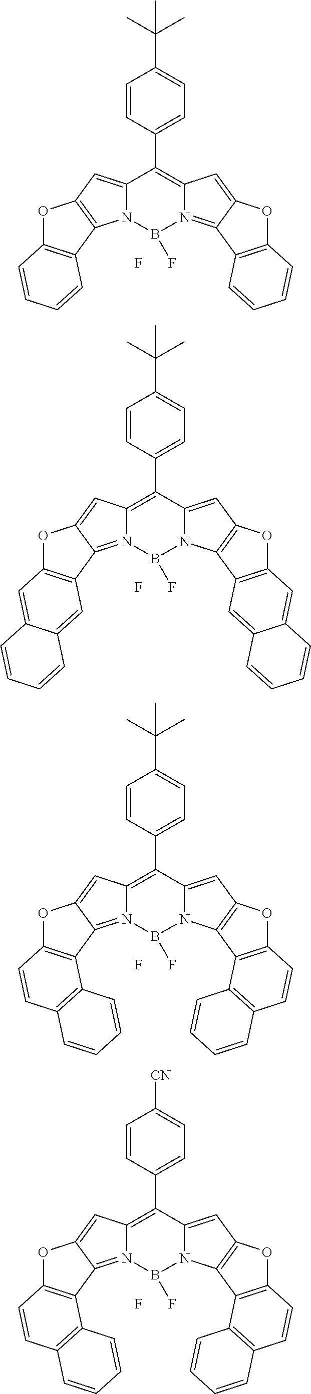

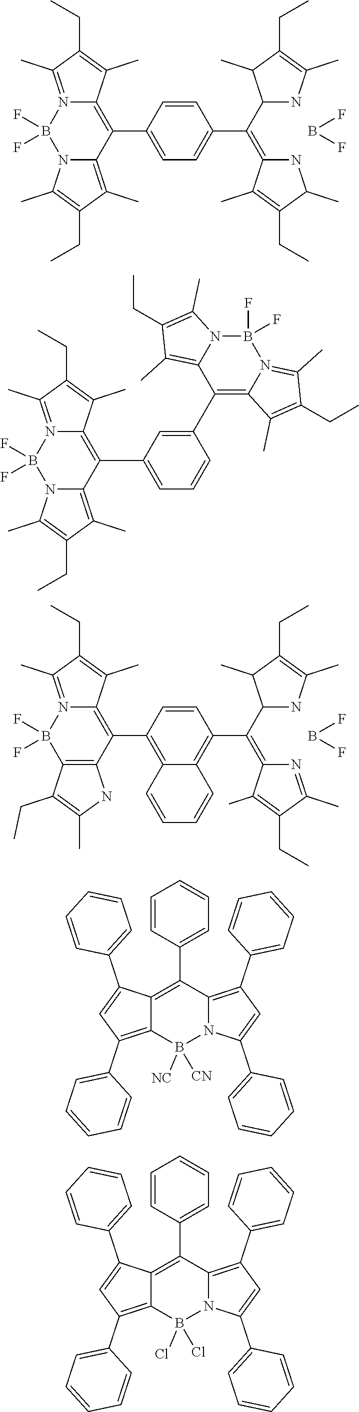

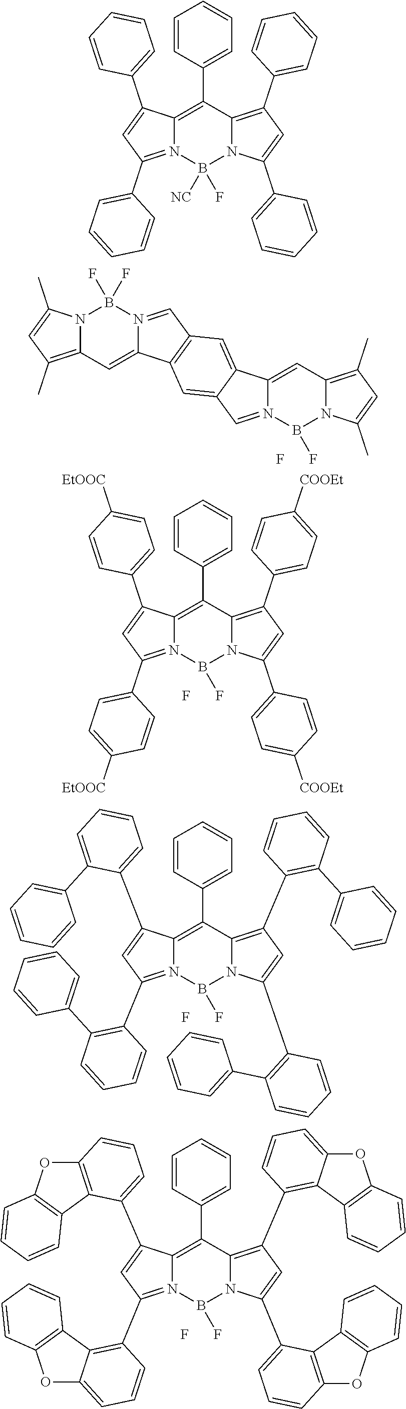

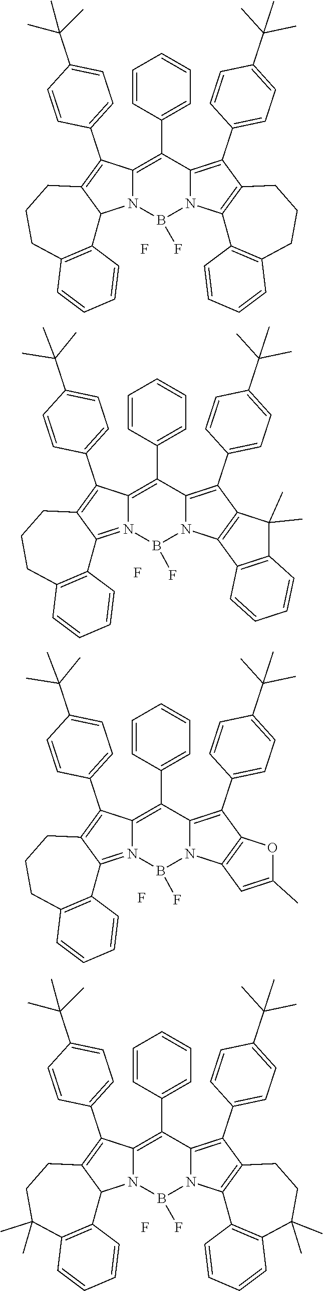

In some embodiments, the emitter E is an inorganic nanoemitter as disclosed in WO Publication No. 2022214031, which is hereby incorporated by reference in its entirety.

In yet another aspect, the present disclosure further provides a formulation comprising at least one compound or polymer or mixture as described herein, at least one organic solvent, and/or an organic resin.

In some embodiments, the formulation comprises an organic resin. In some embodiments, the formulation comprises two or more organic resins. In some embodiments, the formulation comprises three or more organic resins.

For the purposes of the present disclosure, the organic resin refers to a resin prepolymer or a resin formed after the prepolymer is crosslinked or cured.

The organic resins suitable for the present disclosure include, but not limited to: polystyrene, propylamine, polymethyl methacrylate, polycarbonate, polyurethane, polyvinylpyrrolidone, polyvinyl acetate, polybutylene, polyethylene glycol, silicone oil, epoxy resin, polyvinyl alcohol, polyacrylonitrile, polyvinylidene chloride (PVDC), polystyreneacrylonitrile (SAN), polybutylene terephthalate (PBT), polyethylene terephthalate (PET), polyvinyl butyrate (PVB), polyvinyl chloride (PVC), polyamide, polyoxymethylene, polyimide, polyetherimide, and mixtures thereof.

Further, the organic resin suitable for the present disclosure includes, but not limited to, those prepared by the homopolymerization or copolymerization of the following monomers (resin prepolymers): styrene derivatives, acrylate derivatives, acrylonitrile derivatives, acrylamide derivatives, vinyl ester derivatives, vinyl ether derivatives, maleimide derivatives, conjugated diene derivatives.

Examples of styrene derivatives include, but not limited to alkylstyrenes, such as α-methylstyrene, o-, m-, p-methylstyrene, p-butylstyrene; especially p-tert-butylstyrene, alkoxystyrene, such as p-methoxystyrene, p-butoxystyrene, p-tert-butoxystyrene.

Examples of acrylate derivatives include, but not limited to, methyl acrylate, methyl methacrylate, ethyl acrylate, ethyl methacrylate, n-propyl acrylate, n-propyl methacrylate, isopropylacrylate, isopropyl methacrylate, n-butyl acrylate, n-butyl methacrylate, isobutyl acrylate, isobutyl methacrylate, sec-butyl acrylate, sec-butyl methacrylate, tert-butyl acrylate, tert-butyl methacrylate, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyethyl acrylate, 2-methoxyethyl methacrylate, 2-phenoxyethyl acrylate, 2-phenoxyethyl methacrylate, methoxydiethylene glycol acrylate, methoxydiethylene glycol methacrylate, methoxytriethylene glycol acrylate, methoxytriethylene glycol methacrylate, methoxypropylene glycol acrylate, methoxypropylene glycol methacrylate, methoxy dipropylene glycol acrylate, methoxydipropylene glycol methacrylate, isobornyl acrylate, isobornyl methacrylate, dihydrodicyclopentadienyl acrylate, dicyclopentadiene methacrylate, adamantane (meth) acrylate, norbornene (meth) acrylate, 2-hydroxy-3-phenoxypropyl acrylate, 2-hydroxy-3-phenoxypropyl methacrylate, glycerol monoacrylate, glycerol monomethacrylate, 2-aminoethyl acrylate, 2-aminoethyl methacrylate, 2-dimethylaminoethyl acrylate, 2-dimethylaminoethyl methacrylate, N,N-dimethylaminoethyl (meth) acrylic acid, N,N-diethylaminoethyl (meth) acrylate, 2-aminopropyl acrylate, 2-aminopropyl methacrylate, 2-dimethylaminopropyl acrylate, 2-dimethylaminopropyl methacrylate, 3-aminopropyl acrylate, 3-aminopropyl methacrylate, benzyl N,N-dimethyl-1,3-propanediamine(meth)acrylate, 3-dimethylaminopropyl acrylate, 3-dimethylaminopropyl methacrylate, glycidyl acrylate, and glycidyl methacrylate.

Examples of acrylonitrile derivatives include, but not limited to, acrylonitrile, methacrylonitrile, 2-chloroacrylonitrile, and vinylidene cyanide.

Examples of acrylamide derivatives include, but not limited to, acrylamide, methacrylamide, α-chloroacrylamide, N-2-hydroxyethyl acrylamide, and N-2-hydroxyethyl methacrylamide.

Examples of vinyl ester derivatives include, but not limited to, vinyl acetate, vinyl propionate, vinyl butyrate, and vinyl benzoate.

Examples of vinyl ether derivatives include, but not limited to, vinyl methyl ether, vinyl ethyl ether, and allyl glycidyl ether.

Examples of maleimide derivatives include, but not limited to, maleimide, benzylmaleimide, N-phenylmaleimide, and N-cyclohexylmaleimide.

Examples of conjugated diene derivatives include, but not limited to, 1,3-butadiene, isoprene, and 2-chloro-1,3-butadiene.

The homopolymers or copolymers can be prepared by free-radical polymerization, cationic polymerization, anionic polymerization, or organometallic catalysis polymerization (for example Ziegler-Natta catalysis). The polymerization process may be suspension polymerization, emulsion polymerization, solution polymerization, or bulk polymerization.

The number average molecular weight Mn (as determined by GPC) of the organic resins is generally in the range of 10 000 g/mol to 1 000 000 g/mol, preferably in the range of 20 000 g/mol to 750 000 g/mol, more preferably in the range of 30 000 g/mol to 500 000 g/mol.

In some embodiments, the organic resin is a thermosetting resin or an UV curable resin. In some embodiments, the organic resin is cured by a method that will enable roll-to-roll processing.

Thermosetting resins require curing in which they undergo an irreversible process of molecular cross-linking, which makes the resin non-fusible. In some embodiments, the thermosetting resin is an epoxy resin, a phenolic resin, a vinyl ester resin, a melamine co-polycondensation resin, an urea-formaldehyde resin, an unsaturated polyester resin, a polyurethane resin, an allyl resin, an acrylic resin, a polyamide resin, a polyamide-imide resin, a phenol-amide polycondensation resin, an urea-melamine polycondensation resin, or any combination thereof.

In some embodiments, the thermosetting resin is an epoxy resin. The epoxy resins are easy to cure and do not give off volatiles or generate by-products from a wide range of chemicals. The epoxy resins can also be compatible with most substrates and tend to readily wet surfaces. See also Boyle, M. A. et al., “Epoxy Resins”, Composites, Vol. 21, ASM Handbook, pages 78-89 (2001).

In some embodiments, the organic resin is a silicone thermosetting resin. In some embodiments, the silicone thermosetting resin is OE6630A or OE6630B (Dow Corning Corporation (Auburn, Michigan.)).

In some embodiments, the formulation comprises a solvent. In some embodiments, the formulation comprises two or more solvents. In some embodiments, the formulation comprises three or more solvents.

In some embodiments, the formulation as described herein is a solution.

In some embodiments, the formulation as described herein is a dispersion.

The formulation in the embodiments of the present disclosure may comprise the compound of 0.01 wt % to 20 wt %, preferably 0.1 wt % to 20 wt %, more preferably 0.2 wt % to 20 wt %, and most preferably 1 wt % to 15 wt %

Using the formulation as described herein, the color conversion layer may be fabricated by ink-jet printing, transfer printing, photolithography, etc. In this case, the color conversion material needs to be dissolved alone or together with other materials in an organic solvent, to form the ink. The mass concentration of the color conversion material in the ink is not less than 0.1 wt %. The color conversion ability of the color conversion layer can be tuned by adjusting the concentration of the color conversion material in the ink and the thickness of the color conversion layer. In general, the higher the concentration of the color conversion material or the thickness of the layer, the higher the color conversion efficiency of the color conversion layer would be.

Other materials that can be added into the ink include, but not limited to the following materials: polyethylene, polypropylene, polystyrene, polycarbonate, polyacrylate, polyvinyl pyrrolidone, polyvinyl alcohol, polyvinyl acetate, polyethylene glycol, silicone oil, polyacrylonitrile, polyvinyl chloride, polyvinylidene chloride, polyethylene glycol terephthalate, polybutylene terephthalate, polyvinyl butyrate, polyamide, polyformaldehyde, polyimide, poly (ether-ether-ketone), polysulfone, polyarylether, polyaramide, cellulose, modified cellulose, cellulose acetate, nitrocellulose, or any combination thereof.

In some embodiments, the organic solvent is selected from ester, aromatic ketone, aromatic ether, aliphatic ketone, aliphatic ether, borate, phosphorate, or mixtures of two or more of them.

In some embodiments, the suitable and preferred solvents include, but not limited to, aliphatics, alicyclics, aromatics, amines, thiols, amides, nitriles, esters, ethers, polyethers, alcohols, diols, or polyols.

In some embodiments, the alcohol represents a solvent of the suitable class. The preferred alcohol includes alkylcyclohexanol, especially methylated aliphatic alcohol, naphthol, etc.

Examples of the suitable alcohol solvents include, but not limited to, dodecanol, phenyltridecanol, benzyl alcohol, ethylene glycol, 2-methoxyethanol, glycerol, propylene glycol, propyleneglycol monoethyl ether, etc.

The solvent may be used alone or as mixtures of two or more organic solvents.

In some embodiments, the formulation as described herein comprises a compound as described herein and at least one organic solvent, and further comprises another organic solvent. Examples of the another organic solvent include, but not limited to: methanol, ethanol, 2-methoxyethanol, dichloromethane, trichloromethane, chlorobenzene, 1,2-dichlorobenzene, tetrahydrofuran, anisole, morpholine, toluene, o-xylene, m-xylene, p-xylene, 1,4-dioxane, acetone, 2-butanone, 1,2-dichloroethane, 3-phenoxytoluene, 1,1,1-trichloroethane, 1,1,2,2-tetrachloroethane, ethyl acetate, butyl acetate, N,N-dimethylformamide, N,N-dimethylacetamide, dimethyl sulfoxide,

tetraline, decahydronaphthalene, indene, and/or any combination thereof.

In some embodiments, the another organic solvent of the formulation as described herein is selected from aromatic, heteroaromatic, ester, aromatic ketone, aromatic ether, aliphatic ketone, aliphatic ether, alicyclic or olefin compounds, borate, phosphorate, or mixtures of two or more of them.

Examples of aromatic or heteroaromatic-based solvents of the present disclosure include, but not limited to: 1-tetralone, 3-phenoxytoluene, acetophenone, 1-methoxynaphthalene, diisopropylbenzene, amylbenzene, tetraline, cyclohexylbenzene, 1-chloronaphthalene, 1,4-dimethylnaphthalene, 3-isopropylbiphenyl, p-methyl cumene, dipentylbenzene, 1,2-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl)ethane, 2-isopropylnaphthalene, dibenzyl ether, etc.

In some embodiments, the suitable and preferred organic solvents are aliphatics, alicyclics, aromatics, amines, thiols, amides, nitriles, esters, ethers, polyethers.

The another organic solvent may be a cycloalkane, such as decahydronaphthalene.

In some embodiments, the formulation as described herein comprises at least 50 wt % of an alcoholic solvent; preferably at least 80 wt %; particularly preferably at least 90 wt %.

In some embodiments, the particularly suitable solvent for the present disclosure is a solvent having Hansen solubility parameters in the following ranges:

δ_{d} (dispersion force) is in the range of 17.0 MPa^{1/2} to 23.2 MPa^{1/2}, especially in the range of 18.5 MPa^{1/2} to 21.0 MPa^{1/2}.

δₚ (polarity force) is in the range of 0.2 MPa^{1/2} to 12.5 MPa^{1/2}, especially in the range of 2.0 MPa^{1/2} to 6.0 MPa^{1/2}.

δₕ (hydrogen bonding force) is in the range of 0.9 MPa^{1/2} to 14.2 MPa^{1/2}, especially in the range of 2.0 MPa^{1/2} to 6.0 MPa^{1/2}.

In the formulation as described herein, the boiling point parameter should be taken into account when selecting the organic solvents. As used herein, the boiling points of the organic solvents ≥150° C.; preferably ≥180° C.; more preferably >200° C.; further preferably ≥250° C.; and most preferably ≥275° C. or ≥300° C. The boiling points in these ranges are beneficial in terms for preventing nozzle clogging of the inkjet printhead. The organic solvent can be evaporated from solution system to form a functional film.

In some embodiments, the formulation as described herein, where:
1) the viscosity is in the range of 1 cps to 100 cps at 25° C.; and/or
2) the surface tension is in the range of 19 dyne/cm to 50 dyne/cm at 25° C.

In the formulation as described herein, the surface tension parameter of the organic solvent should be taken into account when selecting the organic solvents. The suitable surface tension parameters of the inks are suitable for the particular substrate and particular printing method. For example, for the ink-jet printing, in some embodiments, the surface tension of the organic solvent at 25° C. is in the range of 19 dyne/cm to 50 dyne/cm, preferably in the range of 22 dyne/cm to 35 dyne/cm, and most preferably in the range of 25 dyne/cm to 33 dyne/cm.

In some embodiments, the surface tension of the ink as described herein at 25° C. is in the range of 19 dyne/cm to 50 dyne/cm; preferably in the range of 22 dyne/cm to 35 dyne/cm; and most preferably in the range of 25 dyne/cm to 33 dyne/cm.

In the formulation as described herein, the viscosity parameters of the ink of the organic solvent should be taken into account when selecting the organic solvents. The viscosity can be adjusted by election different methods, such as by the suitable organic solvent and the concentration of functional materials in the ink. In some embodiments, the viscosity of the organic solvent is less than 100 cps, further less than 50 cps, and still further from 1.5 cps to 20 cps. The viscosity herein refers to the viscosity during printing at the ambient temperature that is generally at 15° C.-30° C., further 18° C.-28° C., still further 20° C.-25° C., especially 23° C.-25° C. The resulting formulation will be particularly suitable for ink-jet printing.

In some embodiments, the viscosity of the formulation as described herein at 25° C. is in the range of from about 1 cps to 100 cps; preferably in the range of 1 cps to 50 cps; and most preferably in the range of 1.5 cps to 20 cps.

The ink obtained from the organic solvent satisfying the above-mentioned boiling point parameter, surface tension parameter and viscosity parameter can form a functional film with uniform thickness and composition property.

Salts are difficult to be purified, and the remaining impurities may often influence the optoelectronic performance of the devices. For the purposes of the present disclosure, in some embodiments, the formulation or the mixture as described herein does not comprise any salts, and preferably does not comprise any organic acid salts formed by organic acids and metals. In terms of cost, the present disclosure preferably excludes organic acid salts comprising transition metals or lanthanide elements.

In yet another aspect, the present disclosure further provides an organic functional film comprising a compound or a polymer or a mixture as described herein, or formed by using a formulation as described herein. Preferably, the organic functional film is formed by using a formulation as described herein.

In yet another aspect, the present disclosure further provides a method for preparing the organic functional film, as shown in the following steps:
1) prepare a formulation as described herein;
2) the formulation is coated on a substrate by printing or coating to form a film, where the method of printing or coating is selected from the group consisting of ink-jet printing, nozzle printing, typographic printing, screen printing, dip coating, spin coating, blade coating, roller printing, torsional roll printing, planographic printing, flexographic printing, rotary printing, spray coating, brush or pad printing, and slit die coating;
3) the obtained film is heated at 50° C. and above, optionally in combination with ultraviolet irradiation, to allow the film to undergo a crosslinking reaction and be cured.

The thickness of the organic functional film is generally 50 nm-200 μm, preferably 100 nm-150 μm, more preferably 500 nm-100 μm, further preferably 1 μm-50 μm, and most preferably 1 μm-20 μm.

In some embodiments, the organic functional film has a thickness of 20 nm to 20 μm, preferably less than 15 μm, more preferably less than 10 μm, even more preferably less than 8 μm, particularly preferably less than 6 μm, further preferably less than 4 μm, and most preferably less than 2 μm.

A further purpose of the present disclosure is to provide the use of the compound or the mixture in optoelectronic devices.

In some embodiments, the optoelectronic device may be selected from an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED).

In yet another aspect, the present disclosure further provides an optoelectronic device comprising a compound or a polymer or a mixture or an organic functional film as described herein.

In some embodiments, the optoelectronic device can be selected from an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED).

Preferably, the optoelectronic device is an electroluminescent device, such as an organic light-emitting diode (OLED), an organic light emitting electrochemical cell (OLEEC), an organic light emitting field effect transistor, a perovskite light emitting diode (PeLED), and a quantum dot light emitting diode (QD-LED), where the functional layer comprises a compound or a polymer or a mixture or an organic functional film as described herein. The functional layer may be selected from a hole-injection layer, a hole-transport layer, an electron-injection layer, an electron-transport layer, a light-emitting layer, or a cathode passivation layer (CPL).

In some embodiments, the optoelectronic device is an electroluminescent device, comprising two electrodes, and the functional layer is located on the same side of the two electrodes.

In some embodiments, the optoelectronic device comprises a light emitting unit and a color conversion layer, where the color conversion layer comprises a compound or a polymer or a mixture or an organic functional film.

In some embodiments, the light emitting unit is a solid-state light emitting device. The solid-state light emitting device is preferably selected from a LED, an organic light-emitting diode (OLED), an organic light emitting electrochemical cell (OLEEC), an organic light emitting field effect transistor, a perovskite light emitting diode (PeLED), a quantum dot light emitting diode (QD-LED), or a nanorod LED (see DOI: 10.1038/srep28312).

In some embodiments, the light emitting unit emits blue light, which is converted into green light by the color conversion layer.

In some embodiments, the light emitting unit emits green light, which is converted into yellow light or red light by the color conversion layer.

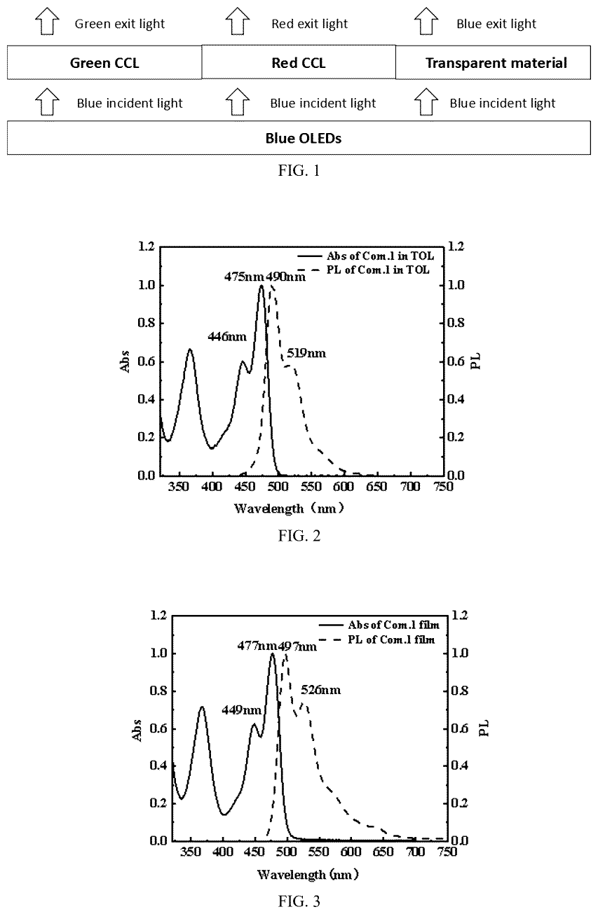

The present disclosure further relates to a display comprising at least three pixels of red, green and blue. As shown in FIG. 1, the blue pixel comprises a blue emitting unit, and the pixel of red or green comprises a blue emitting unit and a corresponding red or green color conversion layer.

In some embodiments, the optoelectronic device is an organic light-emitting device comprising a substrate, a first electrode, an organic light-emitting layer, a second electrode, a color conversion layer, and an encapsulation layer (e.g., an outermost encapsulation layer) in sequence from bottom to top, the second electrode is at least partially transparent, where 1) the color conversion layer comprises a compound or a polymer as described herein, and an emitter E; 2) the color conversion layer at least partially absorbs the light emitted by the organic light-emitting layer through the second electrode; 3) the emission spectrum of the compound or the polymer is on the short wavelength side of the absorption spectrum of the emitter E, and at least partially overlaps with the absorption spectrum of the emitter E; 4) the FWHM of the emission spectrum of the emitter E≤55 nm.

The compound, the emitter E, and the embodiments thereof are as described herein.

In some embodiments, the color conversion layer absorbs 30% or more of the light emitted by the organic light-emitting layer through the second electrode, preferably 40% or more, and most preferably 45% or more.

In some embodiments, the color conversion layer absorbs 90% or more of the light emitted by the organic light-emitting layer through the second electrode, preferably 95% or more, more preferably 99% or more, and most preferably 99.9% or more.

In some embodiments, the thickness of the color conversion layer is between 100 nm and 5 μm, preferably between 150 nm and 4 μm, more preferably between 200 nm and 3 μm, and most preferably between 200 nm and 2 μm.

In some embodiments, the organic light-emitting device is an organic electroluminescent device.

In some embodiments, the organic electroluminescent device is an OLED. More preferably, the first electrode is an anode, the second electrode is a cathode. Particularly preferably, the organic electroluminescent device is a top-emission OLED.

The substrate should be opaque or transparent. A transparent substrate could be used to produce a transparent light-emitting device. The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level/valence band energy level of the emitter of the light-emitting layer or the p-type semiconductor materials of the hole-injection layer (HIL)/hole-transport layer (HTL)/electron-blocking layer (EBL)<0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level/conduction band energy level of the emitter of the light-emitting layer, or the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc. In some embodiments, the transmittance of the cathode in the range of 400 nm-680 nm≥40%, preferably ≥45%, more preferably ≥50%, and most preferably ≥60%. Typically, 10 nm-20 nm of Mg:Ag alloys can be used as transparent cathodes, and the ratio of the Mg:Ag can range from 2:8 to 0.5:9.5.

In the organic electroluminescent device as described herein, the light-emitting layer preferably comprises a blue fluorescent host and a blue fluorescent dopant. In some embodiments, the light-emitting layer comprises a blue phosphorescent host and a blue phosphorescent dopant.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1. The entire contents of these three documents are hereby incorporated herein for reference.

Further, the organic electroluminescent device further comprises a cathode capping layer (CPL).

In some embodiments, the CPL is disposed between the second electrode and the color conversion layer.

In some embodiments, the CPL is disposed on the top of the color conversion layer.

The CPL material generally requires a high refractive index (n), such as n≥1.95@460 nm, n≥1.90@520 nm, n≥1.85@620 nm. Examples of the CPL materials include:

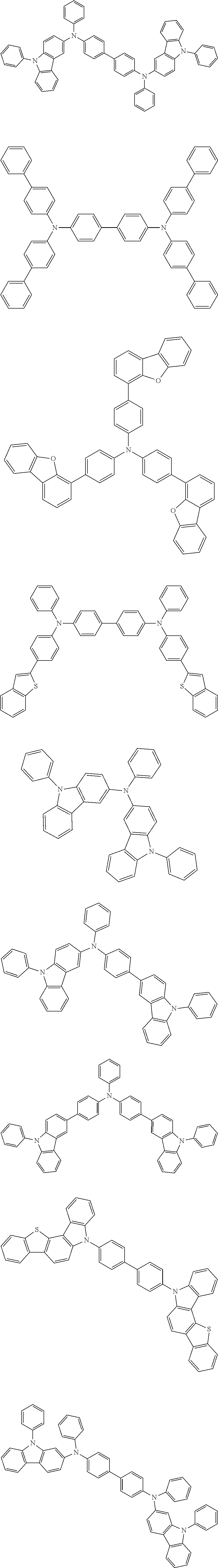

More further examples of the CPL materials those can be found in the following patent publications: KR20140142021A, KR20140142923A, KR20140143618A, KR20140145370A, KR20150012835A, U.S. Pat. No. 9,496,520B2, CN103828485B, CN104752619A, US2016308162A1, U.S. Pat. No. 9,095,033B2, US2014034942A1. The above patent publications are incorporated herein by reference in their entirety.

In some embodiments, the color conversion layer comprises a CPL material as described herein. In some embodiments, the color conversion layer is formed by co-evaporating a CPL material, a compound (i.e., the host material H), and an emitter E as described herein. In some embodiments, the mass ratio of the compound (i.e., the host material H) is in the range of 50% to 20%, and the mass ratio of the emitter E is in the range of 10% to 15%.

Preferably, the encapsulation layer of the organic electroluminescent device is thin-film encapsulated (TFE).

In yet another aspect, the present disclosure further provides a display panel, where at least one pixel comprises an organic electroluminescent device as described herein.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### SPECIFIC EMBODIMENT

### Example 1: Synthesis of Compounds

### 1. Synthesis of Compound 1

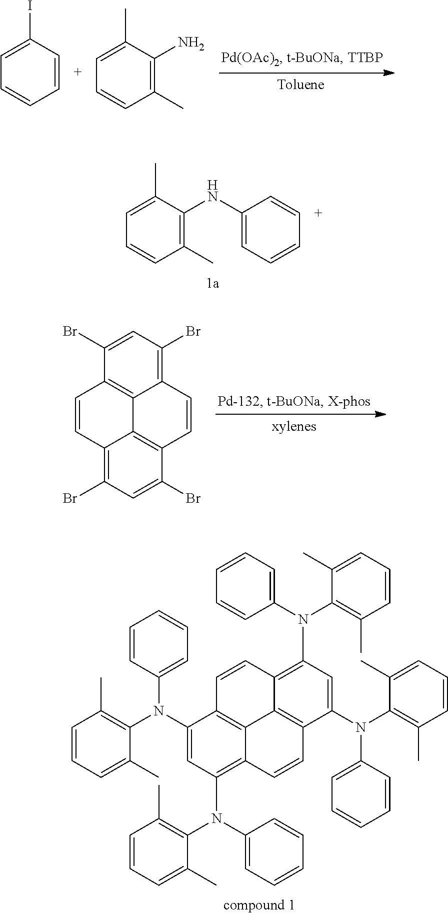

Iodobenzene (50.00 g, 245.1 mmol), 2,6-dimethylaniline (29.68 g, 245.1 mmol), palladium acetate (0.56 g, 2.45 mmol), tri-tert-butylphosphine (1 mL), sodium tert-butoxide (47.06 g, 490.2 mmol), and 500 mL of toluene were added to a 1000 mL dry-clean three-necked flask under N₂ atmosphere. The reaction was heated to 100° C., then refluxed for 5 h. After cooling down to room temperature, the result was extracted with ethyl acetate and saturated brine three times, the organic phase was concentrated and then passed through a short silica gel column (eluent: dichloromethane:n-hexane=1:10), then the result was concentrated to yield 45 g (yield: 93.7%) of intermediate 1a (black-brown solid).

Intermediate la (39.00 g, 197.3 mmol), 1,3,6,8-tetrabromopyrene (22.7 g, 48.3 mmol), Pd-132 (0.62 g, 0.97 mmol), X-Phos (0.62 g), sodium tert-butoxide (16.8 g, 175.3 mmol), and 1 L of xylene were added to a 2000 mL dry-clean three-necked flask under N₂ atmosphere. The reaction was heated to 140° C., then refluxed for 12 h. After cooling down to room temperature, the result was extracted with ethyl acetate and saturated brine three times, the organic phases were concentrated, and the result was redissolved in the hot toluene, then immediately passed through a thermal insulation silica gel column. After the filtrate was concentrated, n-hexane was added to washed the above filtrate. After the filtration, the residue was further washed with n-hexane to obtain 5.1 g of crude product. The obtained crude product was washed with tetrahydrofuran under heating condition, then filtered while hot to yield 1 g (yield: 2.1%) of compound 1 (solid powder).

### 1. Synthesis of Compound 2

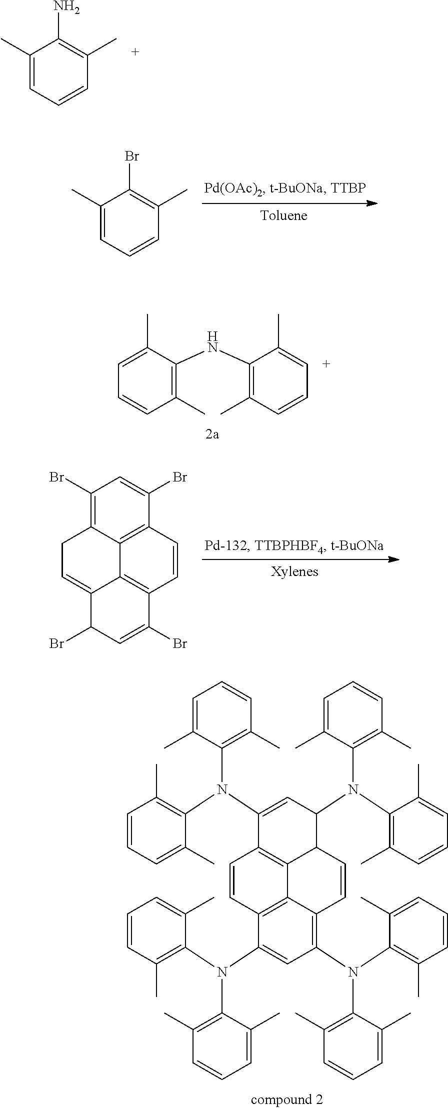

2,6-Dimethylaniline (50.00 g, 413.2 mmol), 2-bromo-m-xylene (76 g, 413.2 mmol), palladium acetate (0.46 g, 2.05 mmol), tri-tert-butylphosphine (1 mL), sodium tert-butoxide (79.3 g, 826.04 mmol), and 500 mL of toluene were added to a 1000 mL dry-clean three-necked flask under N₂ atmosphere. The reaction was heated to 100° C., then refluxed for 2.5 h. After cooling down to room temperature, the result was extracted with ethyl acetate and saturated brine three times, the organic phase was concentrated and then passed through a short silica gel column (eluent: dichloromethane:n-hexane=1:10), then the result was concentrated to yield 88 g (yield: 94.6%) of intermediate 2a (solid).

Intermediate 2a (48.9 g, 217.33 mmol), 1,3,6,8-tetrabromopyrene (25 g, 48.3 mmol), Pd-132 (1.67 g, 2.35 mmol), tri-tert-butylphosphine tetrafluoroborate (1.67 g), sodium tert-butoxide (18.54 g, 193.13 mmol), and 1 L of xylene were added to a 2000 mL dry-clean three-necked flask under N₂ atmosphere. The reaction was heated to 140° C., then refluxed for 12 h. After cooling down to room temperature, the result was extracted with dichloromethane and saturated brine three times, the organic phases were concentrated, and the result was redissolved in the hot toluene, then immediately passed through a thermal insulation silica gel column. After the filtrate was concentrated, n-hexane was added to washed the above filtrate. After the filtration, the residue was further washed with n-hexane to yield 5 g (yield: 9.5%) of compound 2 (solid powder).

### 1. Synthesis of Compound 3

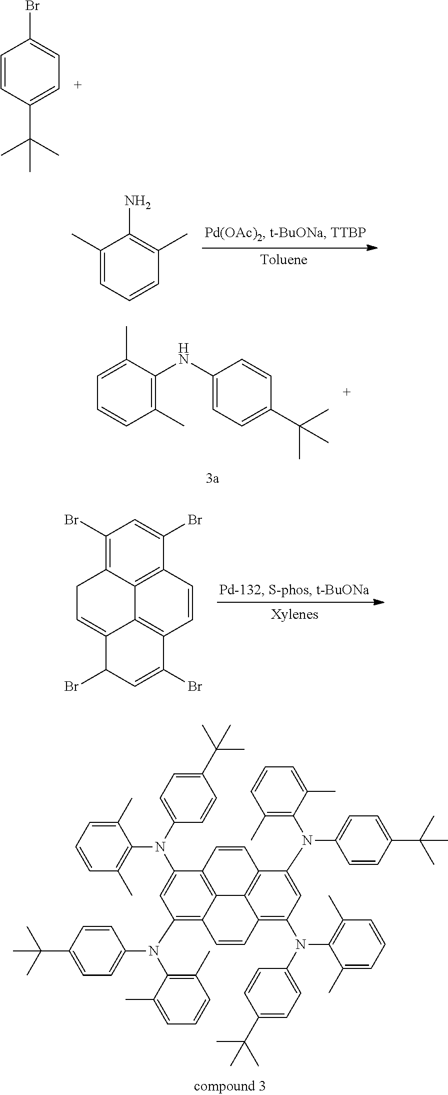

2,6-Dimethylaniline (50.00 g, 413.2 mmol), 1-bromo-4-tert-butylbenzene (88.00 g, 413.2 mmol), palladium acetate (0.46 g, 2.05 mmol), tri-tert-butylphosphine (1 mL), sodium tert-butoxide (59.5 g, 619.83 mmol), and 500 mL of toluene were added to a 1000 mL dry-clean three-necked flask under N₂ atmosphere. The reaction was heated to 100° C., then refluxed for 2.5 h. After cooling down to room temperature, the result was extracted with ethyl acetate and saturated brine three times, the organic phase was concentrated by short silica gel column chromatography (eluent: dichloromethane:n-hexane=1:10), then the result was concentrated to yield 93 g (yield: 89.4%) of intermediate 3a (solid).

Intermediate 3a (43.97 g, 173.79 mmol), 1,3,6,8-tetrabromopyrene (20 g, 38.62 mmol), Pd-132 (0.82 g, 1.16 mmol), S-Phos (0.82 g), sodium tert-butoxide (14.83 g, 154.48 mmol), and 1 L of xylene were added to a 2000 mL dry-clean three-necked flask under N₂ atmosphere. The reaction was heated to 140° C., then refluxed for 12 h. After cooling down to room temperature, the result was extracted with dichloromethane and saturated brine three times, the organic phases were concentrated, and the result was redissolved in the hot toluene, then immediately passed through a thermal insulation silica gel column. After that, the filtrate was concentrated to obtain 25 g of crude product, the obtained crude product was recrystallized with xylene to yield 20 g (yield: 42.8%) of compound 3 (solid powder).

### 4. Synthesis of Compound 4

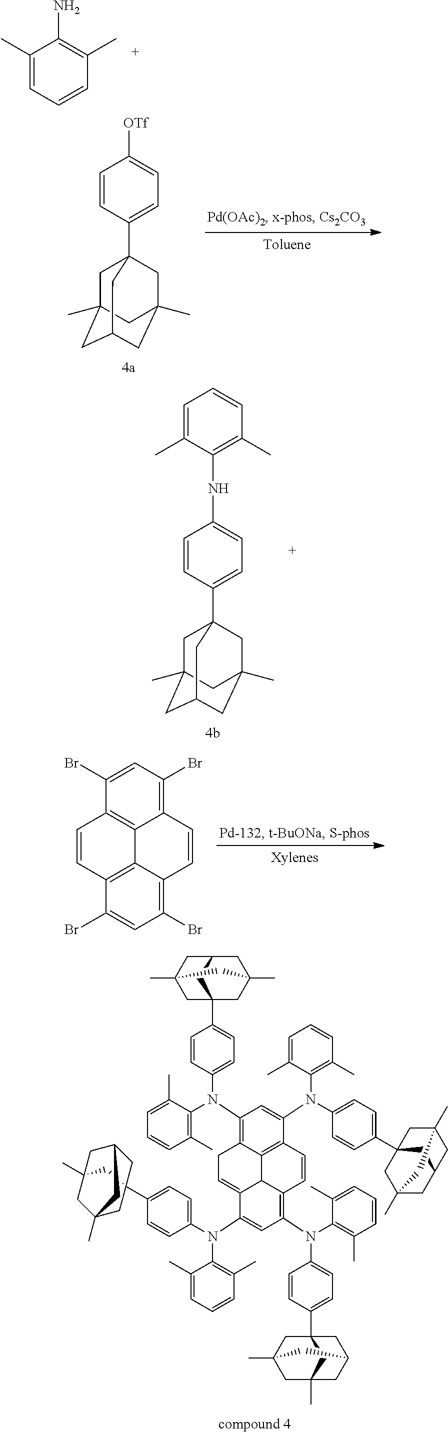

2,6-Dimethylaniline (14.85 g, 122.73 mmol), 4a (50.00 g, 128.87 mmol), palladium acetate (0.28 g, 1.25 mmol), X-Phos (0.28 g), cesium carbonate (59.98 g, 184.09 mmol), and 500 mL of toluene were added to a 1000 mL dry-clean three-necked flask under N₂ atmosphere. The reaction was heated to 100° C., then refluxed for 3.5 h. After cooling down to room temperature, the result was extracted with ethyl acetate and saturated brine three times, the organic phase was concentrated by short silica gel column chromatography (eluent: dichloromethane:n-hexane=1:10), then the result was concentrated to yield 18 g (yield: 40.9%) of intermediate 4b (solid).

Intermediate 4b (18 g, 50.14 mmol), 1,3,6,8-tetrabromopyrene (5.77 g, 11.14 mmol), Pd-132 (0.16 g, 0.23 mmol), S-Phos (0.16 g), sodium tert-butoxide (4.28 g, 44.58 mmol), and 250 mL of xylene were added to a 500 mL dry-clean three-necked flask under N₂ atmosphere. The reaction was heated to 140° C., then refluxed for 14 h. After cooling down to room temperature, the result was extracted with dichloromethane and saturated brine three times, the combined organic phase was concentrated to obtain crude product, then the resulting sample was separated and purified by silica gel column chromatography (eluent: dichloromethane:petroleum ether=1:20) to yield 1.8 g (yield: 9.9%) of compound 4 (solid powder).

### 5. Synthesis of Compound 5

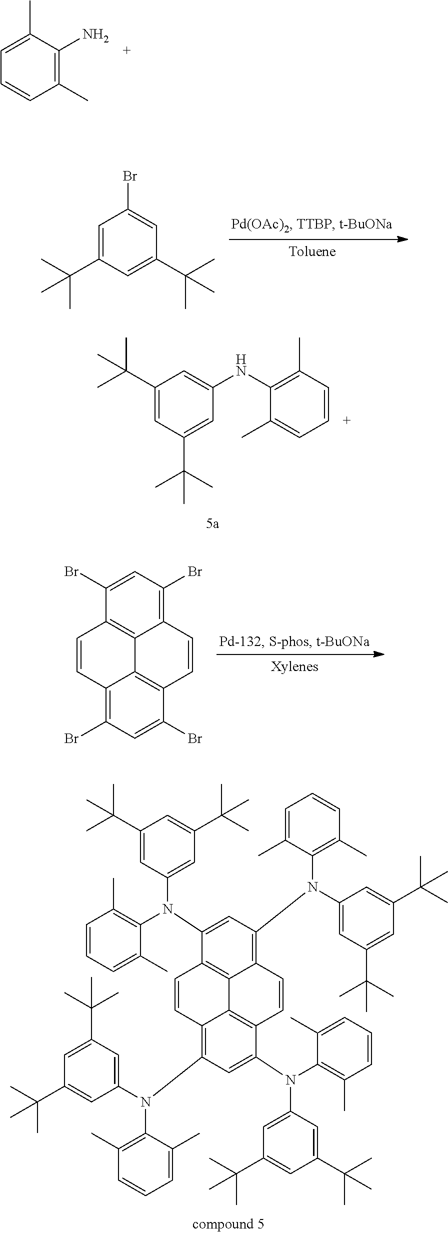

2,6-Dimethylaniline (21 g, 173.55 mmol), 3,5-di-tert-butylbromobenzene (47.00 g, 174.72 mmol), palladium acetate (0.19 g, 0.85 mmol), tri-tert-butylphosphine (1 mL), sodium tert-butoxide (24.09 g, 250.09 mmol), and 500 mL of toluene were added to a 1000 mL dry-clean three-necked flask under N₂ atmosphere. The reaction was heated to 100° C., then refluxed for 12 h. After cooling down to room temperature, the result was extracted with ethyl acetate and saturated brine three times, the organic phase was concentrated and then passed through a short silica gel column (eluent: dichloromethane:n-hexane=1:10), then the result was concentrated to yield 48 g (yield: 89.72%) of intermediate 5a (oily matter).

Intermediate 5a (53 g, 171.52 mmol), 1,3,6,8-tetrabromopyrene (19.74 g, 38.12 mmol), Pd-132 (0.54 g, 0.76 mmol), S-Phos (0.54 g), sodium tert-butoxide (14.64 g, 152.49 mmol), and 1 L of xylene were added to a 2000 mL dry-clean three-necked flask under N₂ atmosphere. The reaction was heated to 140° C., then refluxed for 14 h. After cooling down to room temperature, the result was extracted with dichloromethane and saturated brine three times, the organic phases were concentrated, and the result was redissolved in the hot toluene, then immediately passed through a thermal insulation silica gel column. After that, the filtrate was concentrated to obtain 30 g of crude product, the obtained crude product was recrystallized with tetrahydrofuran to yield 22.5 g (yield: 41.3%) of compound 5 (solid powder).

### 6. Synthesis of Compound 6

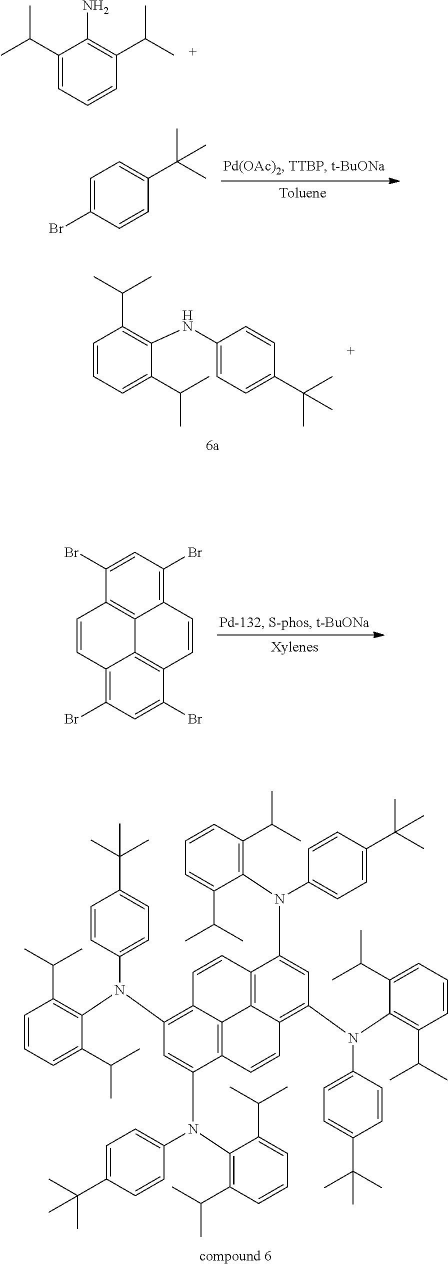 [SMILES: CC(C)c1cccc(C(C)C)c1N ; CC(C)(C)c1ccc(Br)cc1 ; CC(C)c1cccc(C(C)C)c1Nc1ccc(C(C)(C)C)cc1 ; Brc1cc(Br)c2ccc3c(Br)cc(Br)c4ccc1c2c43 ; CC(C)c1cccc(C(C)C)c1N(c1ccc(C(C)(C)C)cc1)c1cc(N(c2ccc(C(C)(C)C)cc2)c2c(C(C)C)cccc2C(C)C)c2ccc3c(N(c4ccc(C(C)(C)C)cc4)c4c(C(C)C)cccc4C(C)C)cc(N(c4ccc(C(C)(C)C)cc4)c4c(C(C)C)cccc4C(C)C)c4ccc1c2c43]

2,6-Diisopropylaniline (24.9 g, 140.68 mmol), 1-bromo-4-tert-butylbenzene (30.00 g, 140.85 mmol), palladium acetate (0.16 g, 0.71 mmol), tri-tert-butylphosphine (1 mL), sodium tert-butoxide (20.3 g, 211.46 mmol), and 500 mL of toluene were added to a 1000 mL dry-clean three-necked flask under N₂ atmosphere. The reaction was heated to 100° C., then refluxed for 12 h. After cooling down to room temperature, the result was extracted with ethyl acetate and saturated brine three times, the organic phase was concentrated by short silica gel column chromatography (eluent: dichloromethane:n-hexane=1:10), then the result was concentrated to yield 38 g (yield: 86.9%) of intermediate 6a (oily matter).

Intermediate 6a (38 g, 122.98 mmol), 1,3,6,8-tetrabromopyrene (14.15 g, 27.33 mmol), Pd-132 (0.39 g, 0.55 mmol), S-Phos (0.39 g), sodium tert-butoxide (10.49 g, 109.27 mmol), and 1 L of xylene were added to a 2000 mL dry-clean three-necked flask under N₂ atmosphere. The reaction was heated to 140° C., then refluxed for 14 h. After cooling down to room temperature, the result was extracted with dichloromethane and saturated brine three times, the combined organic phase was concentrated, then the resulting sample was separated and purified by silica gel column chromatography (eluent: dichloromethane:petroleum ether=1:20) to yield 3.9 g (yield: 10%) of compound 6 (solid powder).

### 7. Synthesis of Compound 7

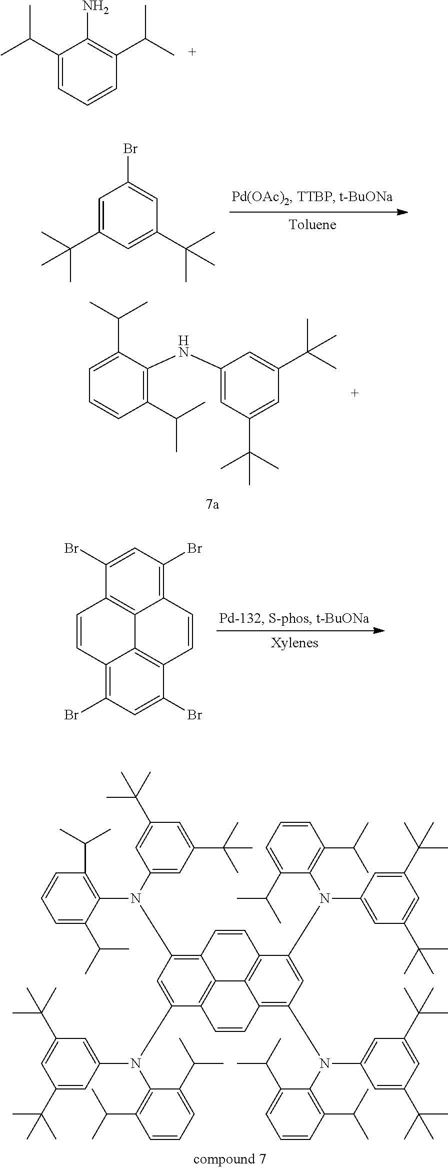

2,6-Diisopropylaniline (40 g, 225.99 mmol), 3,5-di-tert-butylbromobenzene (60.79 g, 226.82 mmol), palladium acetate (0.25 g, 1.11 mmol), tri-tert-butylphosphine (1.5 mL), sodium tert-butoxide (32.5 g, 338.54 mmol), and 500 mL of toluene were added to a 1000 mL dry-clean three-necked flask under N₂ atmosphere. The reaction was heated to 100° C., then refluxed for 12 h. After cooling down to room temperature, the result was extracted with ethyl acetate and saturated brine three times, the organic phase was concentrated by short silica gel column chromatography (eluent: dichloromethane:n-hexane=1:10), then the result was concentrated to yield 76 g (yield: 92.1%) of intermediate 7a (solid).

Intermediate 7a (76 g, 208.22 mmol), 1,3,6,8-tetrabromopyrene (23.96 g, 46.27 mmol), Pd-132 (0.66 g, 0.93 mmol), S-Phos (0.66 g), sodium tert-butoxide (17.77 g, 185.10 mmol.), and 1 L of xylene were added to a 2000 mL dry-clean three-necked flask under N₂ atmosphere. The reaction was heated to 140° C., then refluxed for 14 h. After cooling down to room temperature, the result was extracted with dichloromethane and saturated brine three times, the combined organic phase was concentrated, then the resulting sample was separated and purified by silica gel column chromatography (eluent: dichloromethane:petroleum ether=1:20) to yield 7 g (yield: 9.1%) of compound 7 (solid powder).

### 8. Synthesis of Compound 8

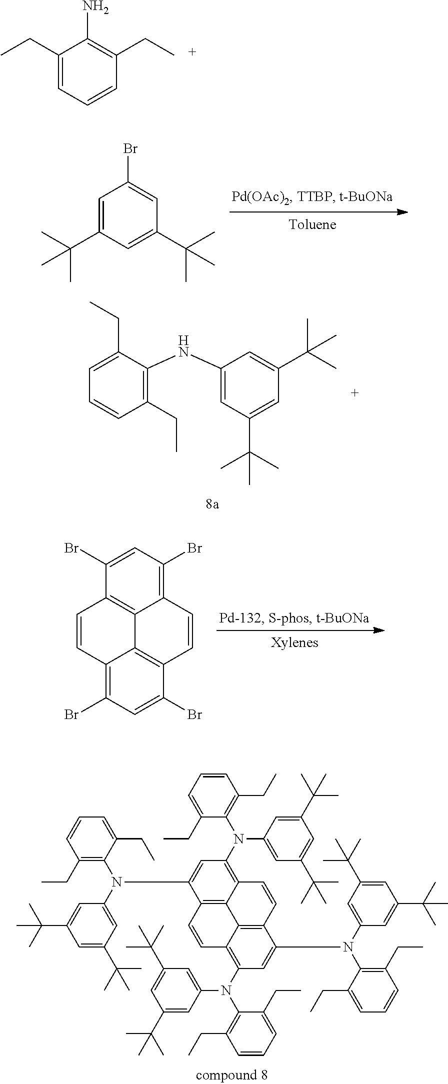

2,6-Diethylaniline (24.9 g, 167.11 mmol), 3,5-di-tert-butylbromobenzene (43 g, 160.45 mmol), palladium acetate (0.19 g, 0.85 mmol), tri-tert-butylphosphine (1 mL), sodium tert-butoxide (24 g, 250.0 mmol), and 500 mL of toluene were added to a 1000 mL dry-clean three-necked flask under N₂ atmosphere. The reaction was heated to 100° C., then refluxed for 12 h. After cooling down to room temperature, the result was extracted with ethyl acetate and saturated brine three times, the combined organic phase was concentrated and then passed through a short silica gel column (eluent: dichloromethane:n-hexane=1:10), then the result was concentrated to yield 52 g (yield: 96.3%) of intermediate 8a (oily matter).

Intermediate 8a (52 g, 154.30 mmol), 1,3,6,8-tetrabromopyrene (17.76 g, 34.30 mmol), Pd-132 (0.49 g, 0.69 mmol), S-Phos (0.49 g), sodium tert-butoxide (13.17 g, 137.19 mmol,), and 1 L of xylene were added to a 2000 mL dry-clean three-necked flask under N₂ atmosphere. The reaction was heated to 140° C., then refluxed for 14 h. After cooling down to room temperature, the result was extracted with dichloromethane and saturated brine three times, the organic phases were concentrated, and the result was redissolved in the hot toluene, then immediately passed through a thermal insulation silica gel column. After that, the filtrate was concentrated to obtain 15 g of crude product, the obtained crude product was recrystallized with tetrahydrofuran to yield 9.6 g (yield: 18.5%) of compound 8 (solid powder).

### 9. Synthesis of Compound 9

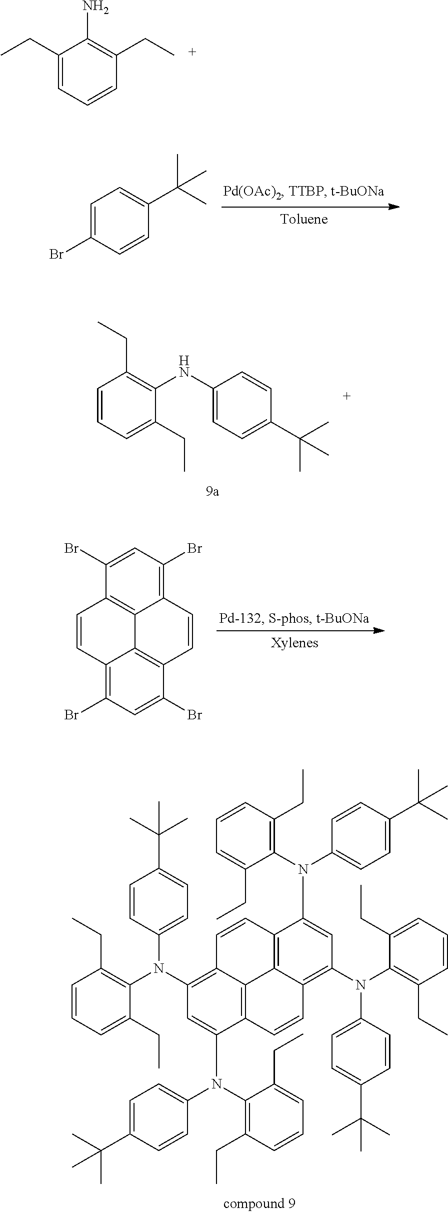 [SMILES: CCc1cccc(CC)c1N ; CC(C)(C)c1ccc(Br)cc1 ; CCc1cccc(CC)c1Nc1ccc(C(C)(C)C)cc1 ; Brc1cc(Br)c2ccc3c(Br)cc(Br)c4ccc1c2c43 ; CCc1cccc(CC)c1N(c1ccc(C(C)(C)C)cc1)c1cc(N(c2ccc(C(C)(C)C)cc2)c2c(CC)cccc2CC)c2ccc3c(N(c4ccc(C(C)(C)C)cc4)c4c(CC)cccc4CC)cc(N(c4ccc(C(C)(C)C)cc4)c4c(CC)cccc4CC)c4ccc1c2c43]

2,6-Diethylaniline (34.98 g, 234.74 mmol), 1-bromo-4-tert-butylbenzene (50 g, 234.74 mmol), palladium acetate (0.26 g, 1.16 mmol), tri-tert-butylphosphine (1.5 mL), sodium tert-butoxide (33.8 g, 352.08 mmol), and 500 mL of toluene were added to a 1000 mL dry-clean three-necked flask under N₂ atmosphere. The reaction was heated to 100° C., then refluxed for 12 h. After cooling down to room temperature, the result was extracted with ethyl acetate and saturated brine three times, the combined organic phase was concentrated and then passed through a short silica gel column (eluent: dichloromethane:n-hexane=1:10), then the solvent was removed to yield 60 g (yield: 90.9%) of intermediate 9a (oily matter).

Intermediate 9a (70 g, 249.11 mmol), 1,3,6,8-tetrabromopyrene (30 g, 57.92 mmol), Pd-132 (0.81 g, 1.14 mmol), S-Phos (0.81 g), sodium tert-butoxide (21.86 g, 227.71 mmol), and 1 L of xylene were added to a 2000 mL dry-clean three-necked flask under N₂ atmosphere. The reaction was heated to 140° C., then refluxed for 14 h. After cooling down to room temperature, the result was extracted with dichloromethane and saturated brine three times, and the result was redissolved in the hot toluene, then immediately passed through a thermal insulation silica gel column. After that, the filtrate was concentrated to obtain 10 g of crude product, the obtained crude product was recrystallized with tetrahydrofuran to yield 4 g (yield: 5.2%) of compound 9 (solid powder).

### Other Materials:

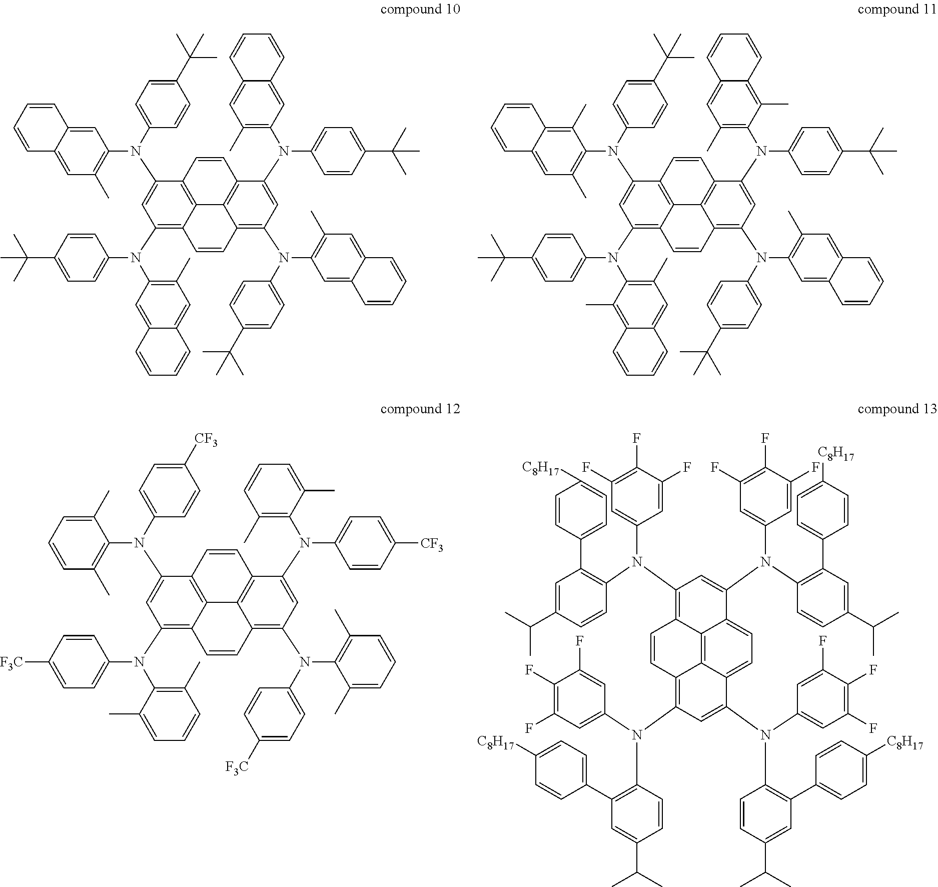

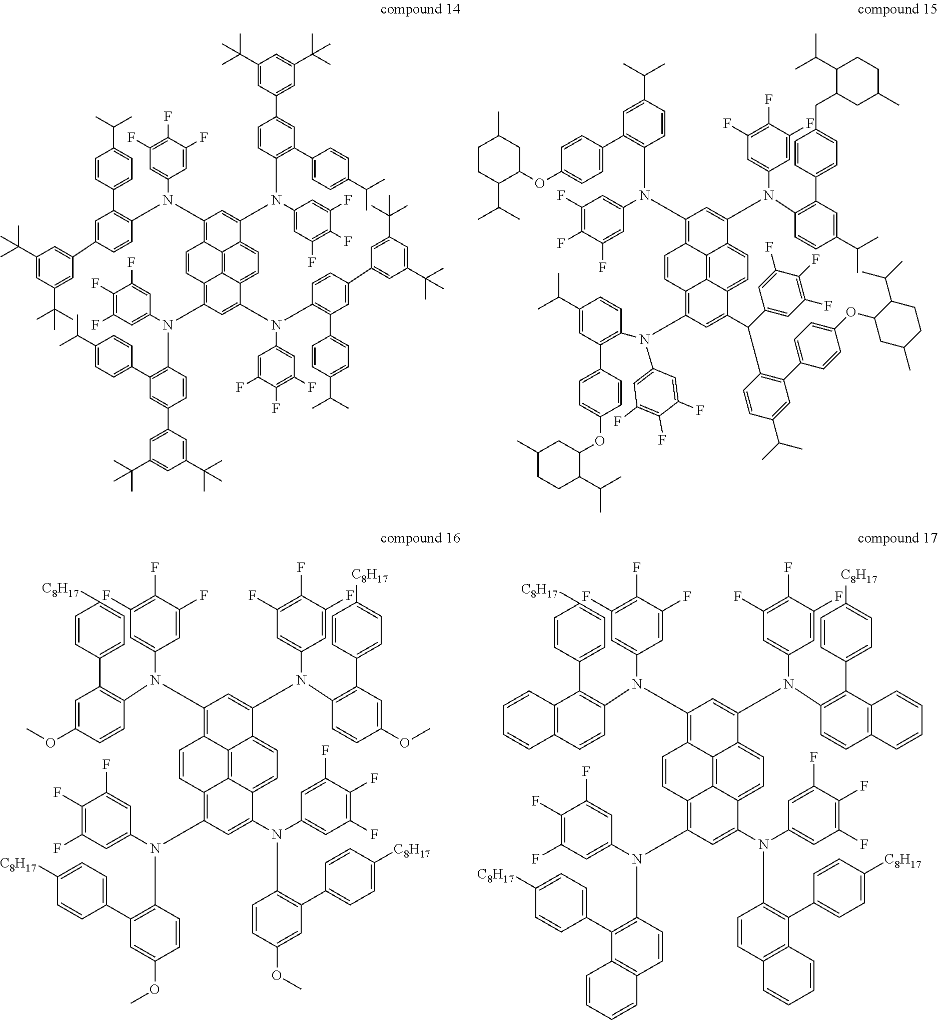

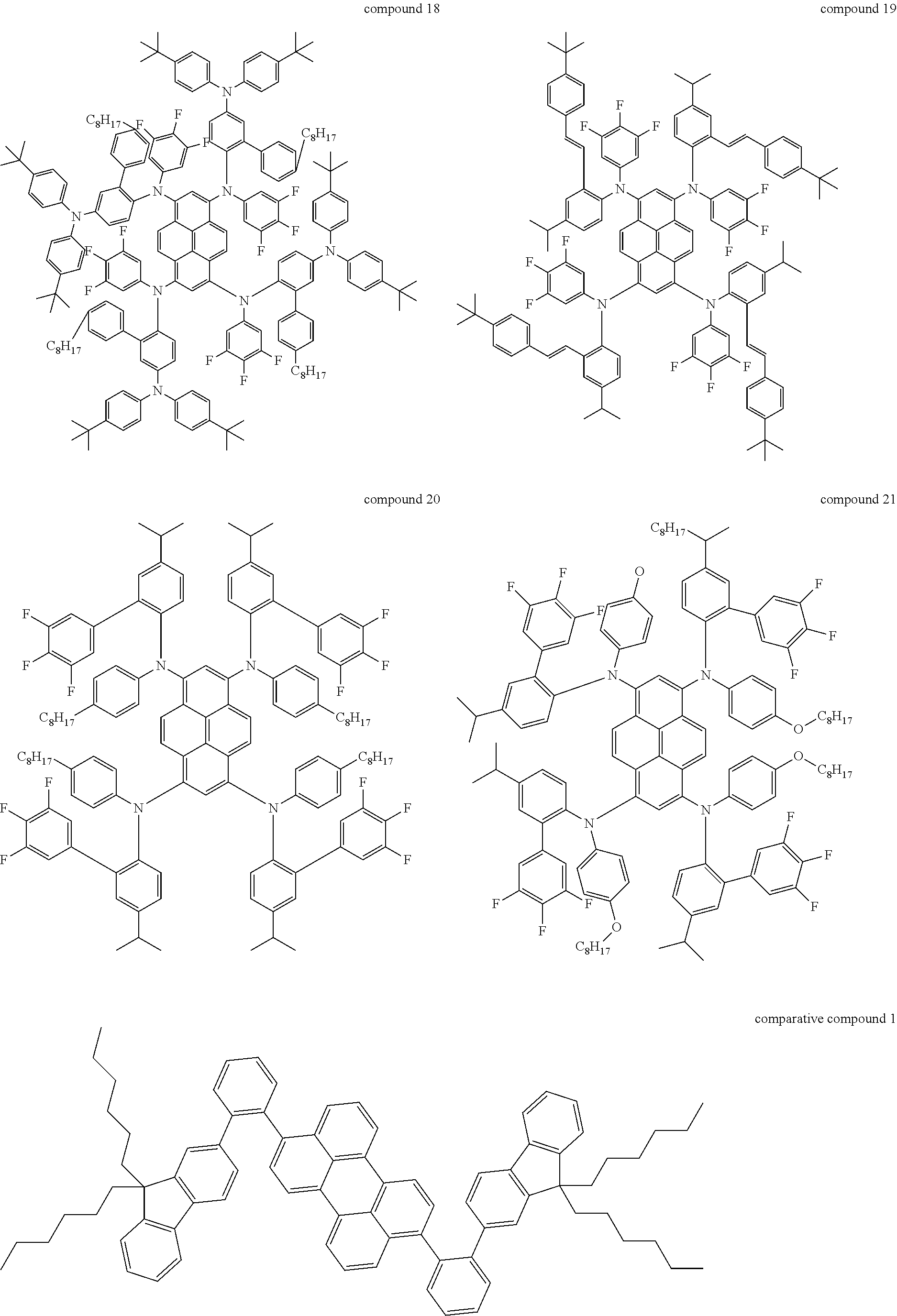

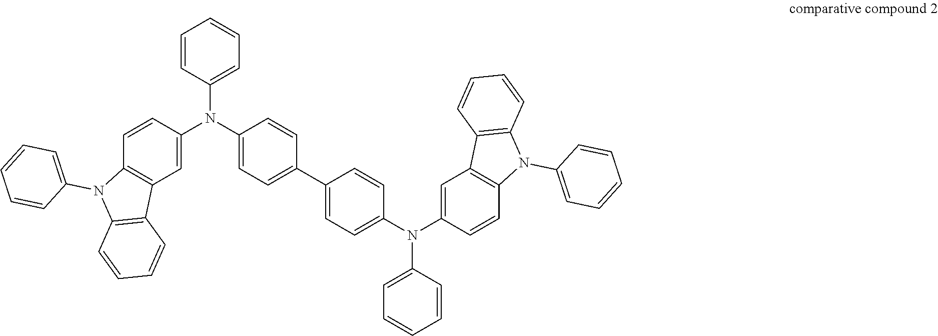 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

The synthesis of compound 10-compound 21 is similar to that of compound 1-compound 9. The comparative compound 1 was synthesized according to patent cooperation treaty (i.e., International Application Publication No. WO2022213993A1), and the comparative compound 2 was synthesized according to US20150069350A1.

| Materials | Target Structures | Raw Materials 1 | Raw Materials 2 | Raw Materials 3 |
|---|---|---|---|---|
| compound 10 | 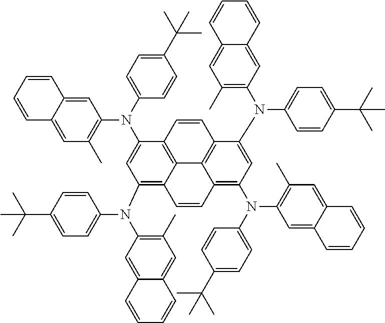 [SMILES: Cc1cc2ccccc2cc1N(c1ccc(C(C)(C)C)cc1)c1cc(N(c2ccc(C(C)(C)C)cc2)c2cc3ccccc3cc2C)c2ccc3c(N(c4ccc(C(C)(C)C)cc4)c4cc5ccccc5cc4C)cc(N(c4ccc(C(C)(C)C)cc4)c4cc5ccccc5cc4C)c4ccc1c2c43] | 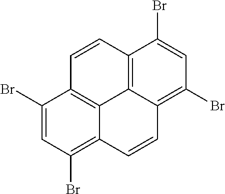 [SMILES: Brc1cc(Br)c2ccc3c(Br)cc(Br)c4ccc1c2c43] | 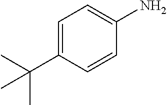 | 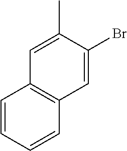 |
| compound 11 | 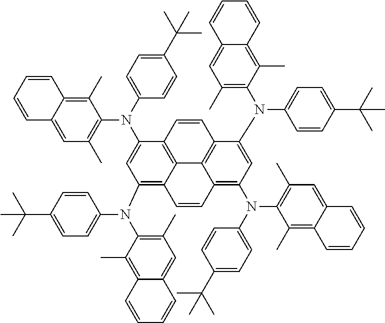 [SMILES: Cc1cc2ccccc2c(C)c1N(c1ccc(C(C)(C)C)cc1)c1cc(N(c2ccc(C(C)(C)C)cc2)c2c(C)cc3ccccc3c2C)c2ccc3c(N(c4ccc(C(C)(C)C)cc4)c4c(C)cc5ccccc5c4C)cc(N(c4ccc(C(C)(C)C)cc4)c4c(C)cc5ccccc5c4C)c4ccc1c2c43] | 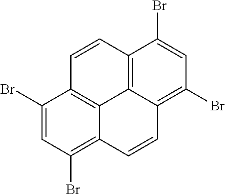 [SMILES: Brc1cc(Br)c2ccc3c(Br)cc(Br)c4ccc1c2c43] | 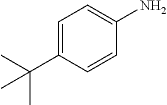 | 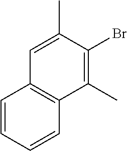 |
| compound 12 | 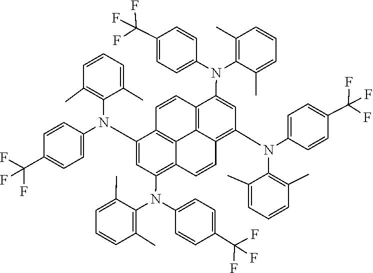 [SMILES: Cc1cccc(C)c1N(c1ccc(C(F)(F)F)cc1)c1cc(N(c2ccc(C(F)(F)F)cc2)c2c(C)cccc2C)c2ccc3c(N(c4ccc(C(F)(F)F)cc4)c4c(C)cccc4C)cc(N(c4ccc(C(F)(F)F)cc4)c4c(C)cccc4C)c4ccc1c2c43] | 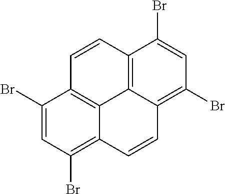 [SMILES: Brc1cc(Br)c2ccc3c(Br)cc(Br)c4ccc1c2c43] | 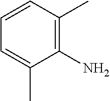 | 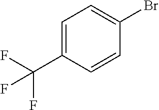 [SMILES: FC(F)(F)c1ccc(Br)cc1] |
| compound 13 | 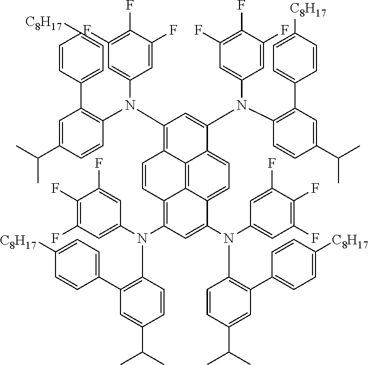 | 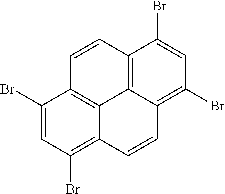 [SMILES: Brc1cc(Br)c2ccc3c(Br)cc(Br)c4ccc1c2c43] | 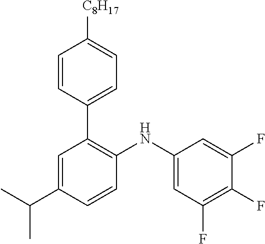 | 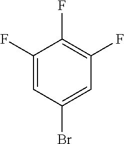 |
| compound 14 | 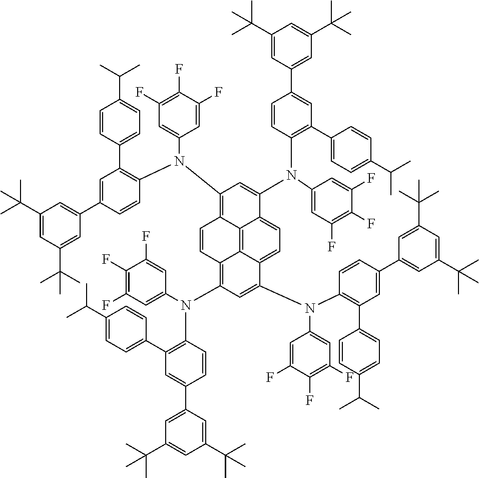 [SMILES: CC(C)c1ccc(-c2cc(-c3cc(C(C)(C)C)cc(C(C)(C)C)c3)ccc2N(c2cc(F)c(F)c(F)c2)c2cc(N(c3cc(F)c(F)c(F)c3)c3ccc(-c4cc(C(C)(C)C)cc(C(C)(C)C)c4)cc3-c3ccc(C(C)C)cc3)c3ccc4c(N(c5cc(F)c(F)c(F)c5)c5ccc(-c6cc(C(C)(C)C)cc(C(C)(C)C)c6)cc5-c5ccc(C(C)C)cc5)cc(N(c5cc(F)c(F)c(F)c5)c5ccc(-c6cc(C(C)(C)C)cc(C(C)(C)C)c6)cc5-c5ccc(C(C)C)cc5)c5ccc2c3c54)cc1] | 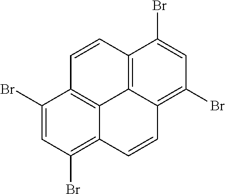 [SMILES: Brc1cc(Br)c2ccc3c(Br)cc(Br)c4ccc1c2c43] | 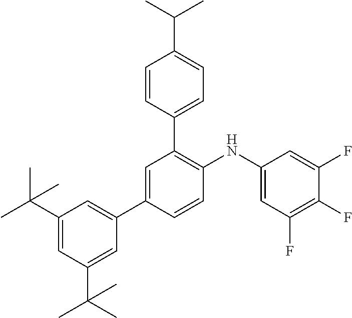 [SMILES: CC(C)c1ccc(-c2cc(-c3cc(C(C)(C)C)cc(C(C)(C)C)c3)ccc2Nc2cc(F)c(F)c(F)c2)cc1] | 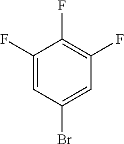 |
| compound 15 | 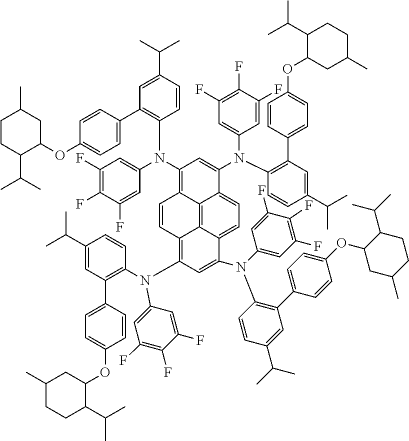 [SMILES: CC1CCC(C(C)C)C(Oc2ccc(-c3cc(C(C)C)ccc3N(c3cc(F)c(F)c(F)c3)c3cc(N(c4cc(F)c(F)c(F)c4)c4ccc(C(C)C)cc4-c4ccc(OC5CC(C)CCC5C(C)C)cc4)c4ccc5c(N(c6cc(F)c(F)c(F)c6)c6ccc(C(C)C)cc6-c6ccc(OC7CC(C)CCC7C(C)C)cc6)cc(N(c6cc(F)c(F)c(F)c6)c6ccc(C(C)C)cc6-c6ccc(OC7CC(C)CCC7C(C)C)cc6)c6ccc3c4c65)cc2)C1] | 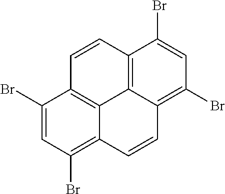 [SMILES: Brc1cc(Br)c2ccc3c(Br)cc(Br)c4ccc1c2c43] | 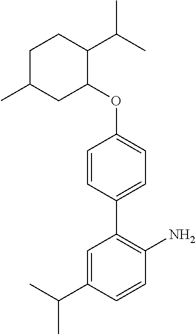 [SMILES: CC1CCC(C(C)C)C(Oc2ccc(-c3cc(C(C)C)ccc3N)cc2)C1] | 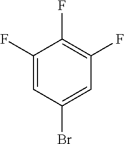 |
| compound 16 | 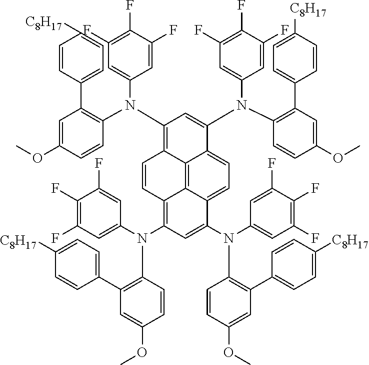 | 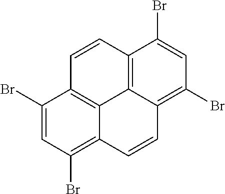 [SMILES: Brc1cc(Br)c2ccc3c(Br)cc(Br)c4ccc1c2c43] | 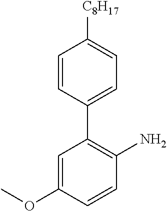 | 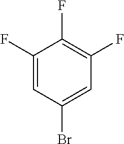 |
| compound 17 | 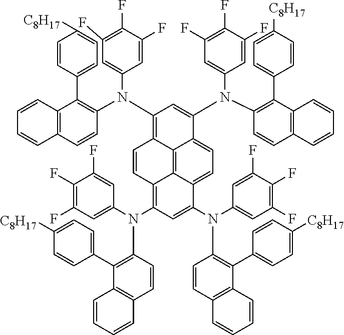 | 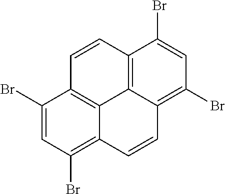 [SMILES: Brc1cc(Br)c2ccc3c(Br)cc(Br)c4ccc1c2c43] | 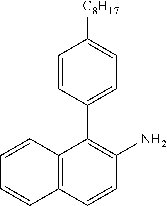 | 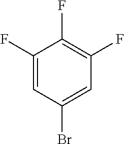 |
| compound 18 | 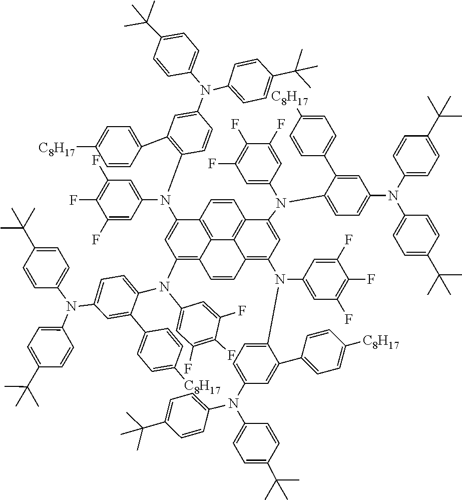 | 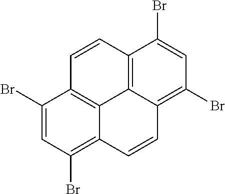 [SMILES: Brc1cc(Br)c2ccc3c(Br)cc(Br)c4ccc1c2c43] | 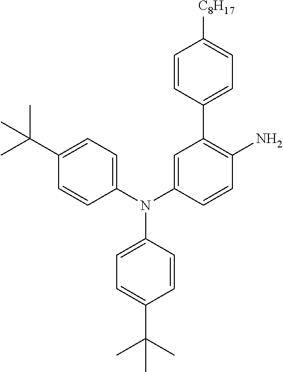 | 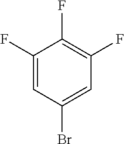 |
| compound 19 | 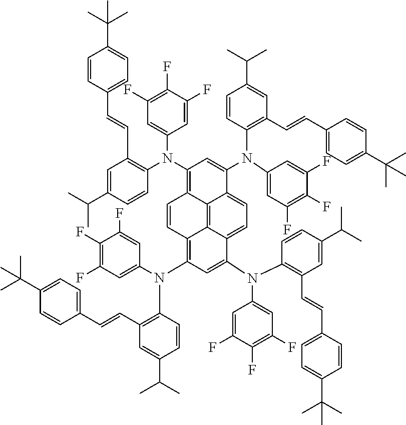 [SMILES: CC(C)c1ccc(N(c2cc(F)c(F)c(F)c2)c2cc(N(c3cc(F)c(F)c(F)c3)c3ccc(C(C)C)cc3/C=C/c3ccc(C(C)(C)C)cc3)c3ccc4c(N(c5cc(F)c(F)c(F)c5)c5ccc(C(C)C)cc5/C=C/c5ccc(C(C)(C)C)cc5)cc(N(c5cc(F)c(F)c(F)c5)c5ccc(C(C)C)cc5/C=C/c5ccc(C(C)(C)C)cc5)c5ccc2c3c54)c(/C=C/c2ccc(C(C)(C)C)cc2)c1] | 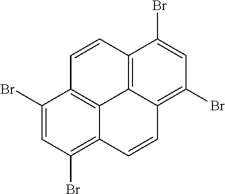 [SMILES: Brc1cc(Br)c2ccc3c(Br)cc(Br)c4ccc1c2c43] | 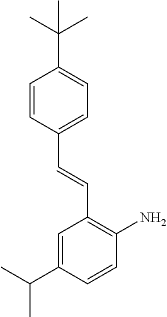 [SMILES: CC(C)c1ccc(N)c(/C=C/c2ccc(C(C)(C)C)cc2)c1] | 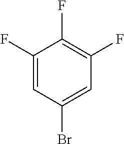 |
| compound 20 | 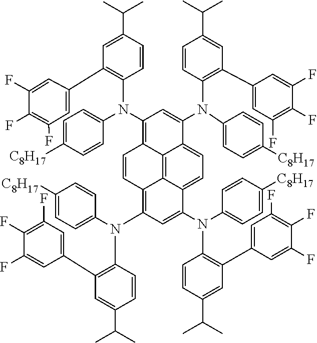 | 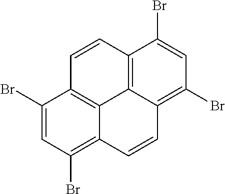 [SMILES: Brc1cc(Br)c2ccc3c(Br)cc(Br)c4ccc1c2c43] | 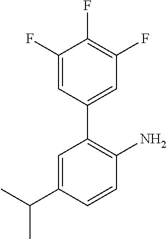 [SMILES: CC(C)c1ccc(N)c(-c2cc(F)c(F)c(F)c2)c1] | 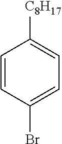 |
| compound 21 | 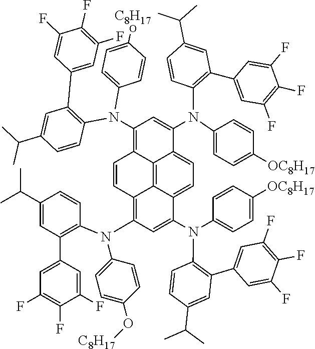 | 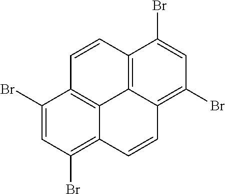 [SMILES: Brc1cc(Br)c2ccc3c(Br)cc(Br)c4ccc1c2c43] | 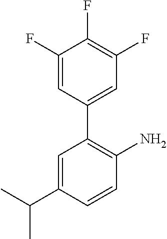 [SMILES: CC(C)c1ccc(N)c(-c2cc(F)c(F)c(F)c2)c1] | 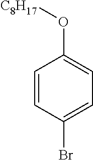 |

22. Synthesis of Polymer P1, where x:y=1:10

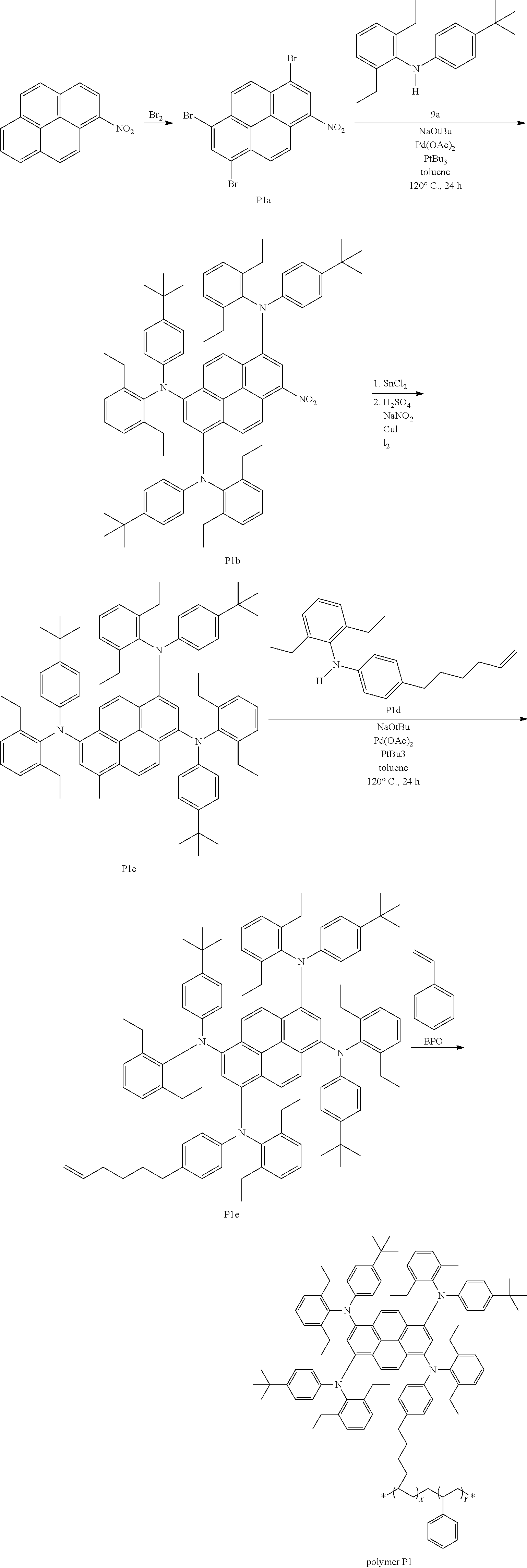

1-Nitropyrene (10 g, 40.44 mmol) and 500 mL of DCM were added to a 1000 mL dry-clean three-necked flask, then bromine (9.69 g, 121.3 mmol) was added under N₂ atmosphere in the dark. The mixture was reacted for 12 h in the dark. The organics were extracted with dichloromethane and saturated brine three times, the solvent was removed by rotary evaporation, and then the residue was recrystallized with toluene to yield 15 g (yield: 76.2%) of intermediate P1a (solid powder).

Intermediate P1a (15 g, 31.05 mmol), intermediate 9a (41.55 g, 139.73 mmol), Pd(OAc)₂ (2.325 mmol), PtBu₃ (3.105 mmol), NaOtBu (26.85 g, 279.45 mmol), and 1 L of toluene were added to a 2000 mL dry-clean three-necked flask under N₂ atmosphere. The mixture was heated to 120° C., refluxed for 24 h, then cooled down to room temperature. The mixture was extracted with dichloromethane and saturated brine three times, the combined organic phase was concentrated, and the resulting crude was purified by short silica gel column chromatography (eluent: ethyl acetate:n-hexane=1:20). Then the solvent was removed to yield 24 g (yield: 71.2%) of intermediate P1b.

Intermediate P1b (24 g, 22.11 mmol), SnCl₂ (20.89 g, 110.54 mmol), and 1.5 L of ethanol absolute were added to a 2000 mL dry-clean three-necked flask under N₂ atmosphere, the mixture was heated to 70° C. and reacted for 1 h. The mixture was poured into the ice water and sodium bicarbonate was added to make the solution slightly alkaline. After the filtration, the solid was vacuum-dried, then dissolved in 500 mL of ethanol absolute. After cooling down to −5° C., 200 mL of H₂SO₄ solution of NaNO₂ (2.42 g, 28.4 mmol) was slowly added to the above mixture with stirring, then a mixture of CuI (0.54 g, 2.84 mmol) and I₂ (3.6 g, 28.4 mmol) was added slowly in batches. After the filtration and drying, 5 g of intermediate P1c was obtained (yield: 19.4%).

Intermediate P1c (5 g, 4.25 mmol), intermediate P1d (2.1 g, 6.5 mmol), Pd(OAc)₂ (0.075 g), PtBu₃ (0.1 g), NaOtBu (1.25 g, 12.75 mmol), and 500 mL of toluene were added to a 1000 mL dry-clean three-necked flask under N₂ atmosphere. The mixture was heated to 120° C., refluxed for 24 h, then cooled down to room temperature. After extracting with dichloromethane and saturated brine three times, the combined organic phase was passed through a short silica gel column (eluent: ethyl acetate:n-hexane=1:20), then the solvent was removed to yield 3.5 g (yield: 61.2%) of intermediate P1e.

Intermediate P1e (3.5 g, 2.6 mmol), styrene (2.70 g, 26 mmol), BPO (0.0624 g, 0.26 mmol), and 100 mL of DCM were added to a 250 mL dry-clean three-necked flask under N₂ atmosphere, then stirred. After being irradiated with UV for 12 h, the monomer was removed by dialysis, then the result was dried to yield 1.56 g (yield: 25.1%) of polymer P1.

The structures of E1, E2, and E3 as the green dopants are as follows, where E1 was purchased from Shanghai Macklin Biochemical Technology Co., Ltd.; E2 was synthesized with reference to WO Publication No. 2024104383; E3 was synthesized with reference to WO Publication No. 2024104383.

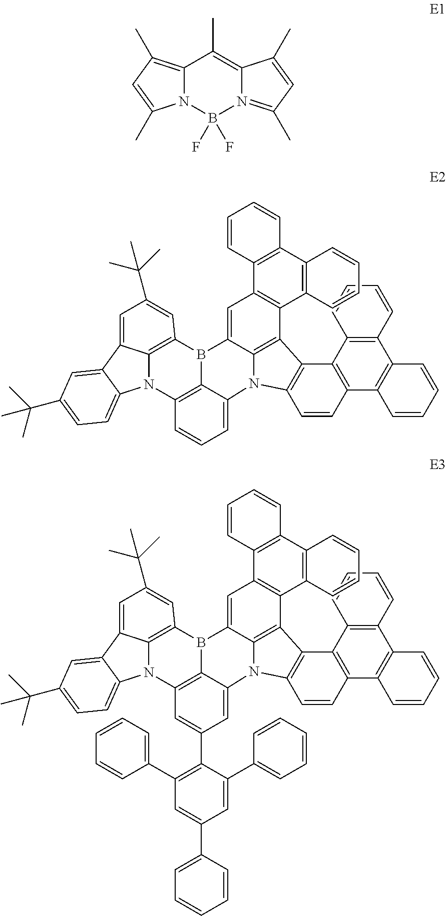

### Example 2: Energy Structures of the Compounds

The energy level of the organic material can be calculated by quantum computation, for example, using TD-DFT (time-dependent density functional theory) by Gaussian 09W (Gaussian Inc.), the specific simulation methods of which can be found in WO2011141110. Firstly, the molecular geometry is optimized by density functional theory “Ground State/DFT/Default Spin/B3LYP” and the basis set “6-31G (d)” (Charge 0/Spin Singlet), then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO energy levels are calculated using the following calibration formula, where S1 and T1 are used directly.

$$HOMO ( eV ) = ( ( HOMO ( G ) \times 27.212 ) - 0.9899 ) / 1.1206 \\ LUMO ( eV ) = ( ( LUMO ( G ) \times 27.212 ) - 2.0041 ) / 1.385$$

Where HOMO(G) and LUMO(G) are the direct calculation results of Gaussian 09W, in units of Hartree. The results are shown in Table 1 below:

TABLE 1

| | HOMO | | | | |
|---|---|---|---|---|---|
| | Corr. | LUMO Corr. | ΔHOMO | ΔLUMO | |
| Materials | [eV] | TeV] | Corr. [eV] | Corr. [eV] | f(S1) |
| compound 1 | −4.87 | −2.61 | 0.69 | 0.55 | 0.6979 |
| compound 2 | −5.08 | −2.55 | 0.31 | 0.58 | 0.0716 |
| compound 3 | −4.85 | −2.61 | 0.58 | 0.60 | 0.6376 |
| compound 4 | −4.83 | −2.57 | 0.56 | 0.57 | 0.7243 |
| compound 5 | −4.78 | −2.52 | 0.60 | 0.56 | 0.6819 |
| compound 6 | −4.73 | −2.51 | 0.68 | 0.52 | 0.6972 |
| compound 7 | −4.67 | −2.46 | 0.72 | 0.53 | 0.6641 |
| compound 8 | −4.73 | −2.49 | 0.65 | 0.54 | 0.6695 |
| compound 9 | −4.86 | −2.61 | 0.56 | 0.62 | 0.6107 |
| compound 10 | −5.02 | −2.74 | 0.41 | 0.52 | 0.6428 |
| compound 11 | −4.89 | 2.65 | 0.54 | 0.43 | 0.6965 |
| compound 12 | −5.34 | −2.94 | 0.63 | 0.51 | 0.8657 |
| compound 13 | −5.22 | −2.90 | 0.51 | 0.59 | 0.6241 |
| compound 14 | −5.02 | −2.75 | 0.69 | 0.46 | 0.6804 |
| compound 15 | −5.04 | −2.76 | 0.64 | 0.53 | 0.4870 |
| compound 16 | −4.99 | −2.72 | 0.60 | 0.52 | 0.4983 |
| compound 17 | −5.22 | −2.92 | 0.51 | 0.38 | 0.5749 |
| compound 18 | −5.09 | −2.94 | 0.17 | 0.57 | 0.5797 |
| compound 19 | −5.05 | −2.79 | 0.66 | 0.24 | 0.3774 |
| compound 20 | −4.74 | −2.56 | 0.68 | 0.38 | 0.5214 |
| compound 21 | −4.62 | −2.48 | 0.62 | 0.35 | 0.5267 |
| comparative | −5.30 | −2.92 | 0.62 | 0.67 | 0.5984 |
| compound 1 | | | | | |
| comparative | −4.95 | −2.11 | 0.32 | 0.00 | 0.0853 |
| compound 2 | | | | | |
| E1 | −5.75 | −3.17 | 1.01 | 2.37 | 0.4938 |
| E2 | −5.53 | −3.04 | 0.26 | 0.58 | 0.4805 |
| E3 | −5.57 | −3.09 | 0.24 | 0.59 | 0.5674 |

### Example 3: Solubility of Compounds

The solubility of the compounds in the toluene were determined as follows:
1. 1000 mg of toluene solution was placed in the transparent glass sample bottle.
2. A certain mass of the compound was added into the toluene, and oscillated until completely dissolved.
3. After the compound was completely dissolved, a certain mass of the compound was further added into the toluene, and oscillated.
4. The above steps were repeated until the compound wasn't dissolved any more.
5. The total mass of the compound added was recorded and the solubility was obtained by calculation.

The solubility of compound 1-compound 21 and E1-E3 in the toluene are shown in Table 2 below.

TABLE 2

| | Solubilities(/100 | | | Solubilities(/100 | |
|---|---|---|---|---|---|
| Materials | g, toluene) | | Materials | g, toluene) | |
| compound 1 | 1.5 | g | compound 13 | >15 | g |
| compound 2 | >2 | g | compound 14 | >5 | g |
| compound 3 | 4.2 | g | compound 15 | >10 | g |
| compound 4 | 7.7 | g | compound 16 | >10 | g |
| compound 5 | 4.6 | g | compound 17 | >4 | g |
| compound 6 | >20 | g | compound 18 | >10 | g |
| compound 7 | >20 | g | compound 19 | >5 | g |
| compound 8 | 30.1 | g | compound 20 | >10 | g |
| compound 9 | 25.9 | g | compound 21 | >10 | g |
| compound 10 | >2.5 | g | E1 | >1 | g |
| compound 11 | >2 | g | E2 | >0.7 | g |
| compound 12 | >4 | g | E3 | >0.7 | g |

### Example 4: Absorption Spectrum, Emission Spectrum and Extinction Coefficients of Compounds (Comparison Between Solution and Film)

The extinction coefficients of the compounds and their absorption and emission spectrums in the solution were determined as follows:
1. The compound with a certain amount of substance concentration in the toluene was prepared in a volumetric flask.
2. The absorption spectrum of the solution was measured by using an ultraviolet-visible spectrophotometer (Puxi T9s) to obtain the absorption peak wavelength.
3. The absorbance of the maximum absorption peak divided by the amount of substance concentration is the molar extinction coefficient of the compound.
4. The emission spectrum of the solution was measured by using a fluorescent spectroscope (Hitachi, F-4700 FL Spectrophotometer) to obtain the emission peak wavelength.

The absorption and emission spectrums of the compounds in the film were determined as follows:
1. The compound with a certain concentration in the toluene was prepared.
2. The prepared toluene solution was dropped onto a clean glass substrate.
3. Spin-coating, and then heating at the 80° C. for 1 min.
4. The absorption spectrum of the film was measured by using an ultraviolet-visible spectrophotometer (Puxi T9s) to obtain the absorption peak wavelength.
5. The emission spectrum of the film was measured by using a fluorescent spectroscope (Hitachi, F-4700 FL Spectrophotometer) to obtain the emission peak wavelength.

The extinction coefficients of the compounds and their absorption and emission peaks in the solution and film are shown in Table 3.

TABLE 3

| | | | | | Molar | | | | |
|---|---|---|---|---|---|---|---|---|---|
| | | | | | extinction | | | | |
| | Absorption | | Emission | | coefficient | | | | |
| | peak | | peak | | of the | | | | |
| | of the | | of the | | toluene | Absorption | | Emission | |
| | toluene | | toluene | | solution/(L · mo | peak of | | peak of | |
| Materials | solution | | solution | | 1⁻¹) | the film | | the film | |
| compound 1 | 475 | nm | 490 | nm | 6.0 × 10⁴ | 477 | nm | 497 | nm |
| compound 3 | 477 | nm | 495 | nm | 3.0 × 10⁴ | 479 | nm | 497 | nm |
| compound 4 | 479 | nm | 498 | nm | 5.5 × 10⁴ | 478 | nm | 497 | nm |
| compound 5 | 477 | nm | 497 | nm | 5.6 × 10⁴ | 476 | nm | 497 | nm |
| compound 8 | 484 | nm | 501 | nm | 5.3 × 10⁴ | 482 | nm | 499 | nm |
| compound 9 | 482 | nm | 500 | nm | 5.3 × 10⁴ | 482 | nm | 499 | nm |
| compound 13 | 473 | nm | 494 | nm | 5.9 × 10⁴ | 473 | nm | 490 | nm |
| compound 14 | 475 | nm | 494 | nm | 5.1 × 10⁴ | 471 | nm | 493 | nm |
| compound 15 | 476 | nm | 493 | nm | 6.8 × 10⁴ | 473 | nm | 491 | nm |
| compound 16 | 475 | nm | 496 | nm | 7.0 × 10⁴ | 476 | nm | 496 | nm |
| compound 17 | 475 | nm | 494 | nm | 5.8 × 10⁴ | 476 | nm | 497 | nm |
| compound 18 | 492 | nm | 520 | nm | 5.1 × 10⁴ | 487 | nm | 518 | nm |
| compound 20 | 485 | nm | 504 | nm | 5.5 × 10⁴ | 486 | nm | 504 | nm |
| comparative | 461 | nm | 485 | nm | 4.3 × 10⁴ | 464 | nm | 543 | nm |
| compound 1 | | | | | | | | | |

| | | | | | | | |
|---|---|---|---|---|---|---|---|
| E1 | 500 | nm | 519 | nm | 6.1 × 10⁴ | — | — |
| E2 | 507 | nm | 523 | nm | 4.8 × 10⁴ | — | — |
| E3 | 512 | nm | 530 | nm | 6.6 × 10⁴ | — | — |

As shown in the table above, the measured compounds exhibit high molar extinction coefficients.

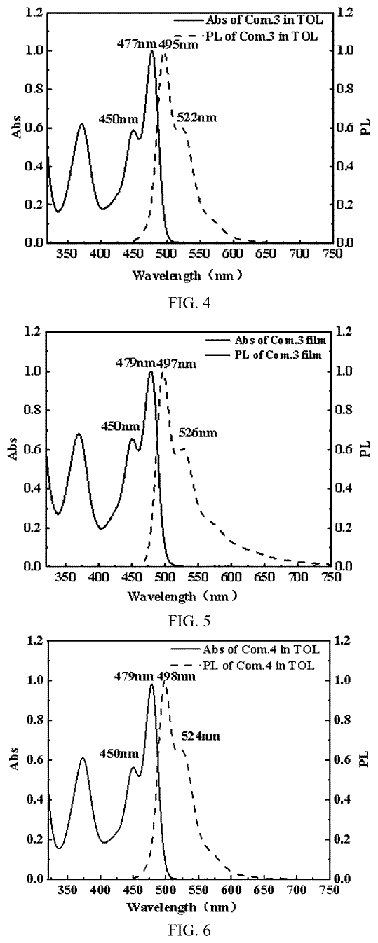

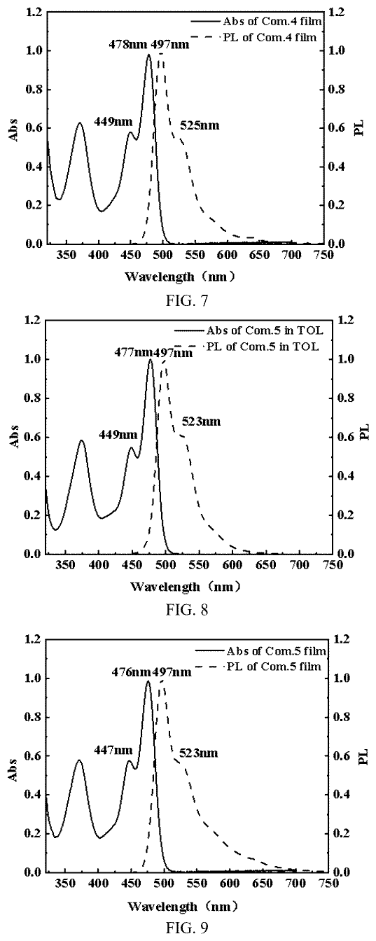

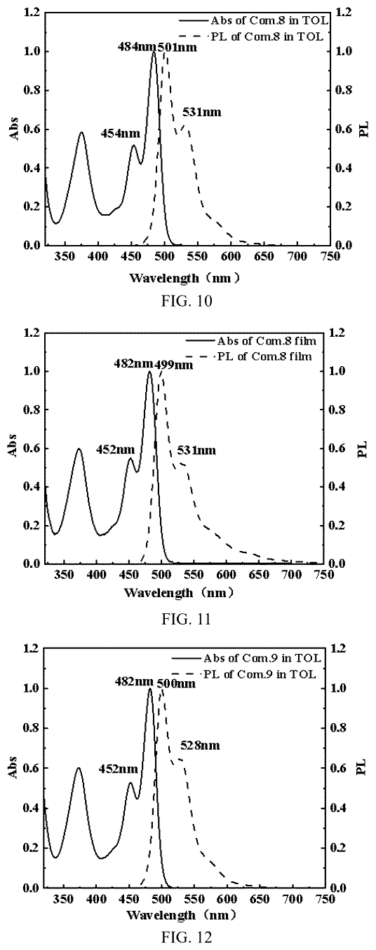

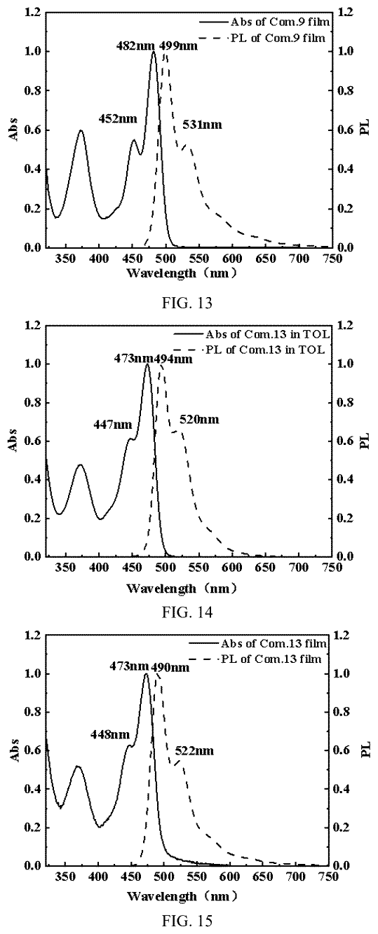

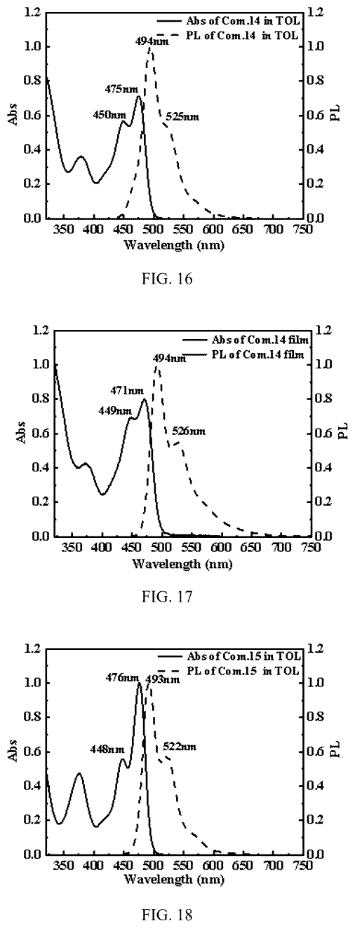

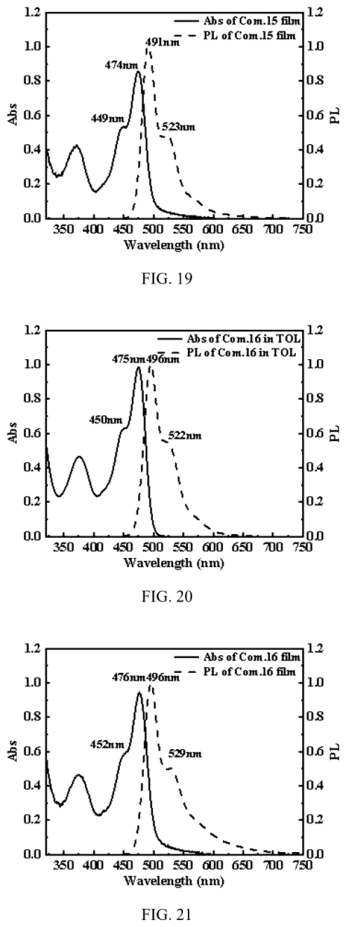

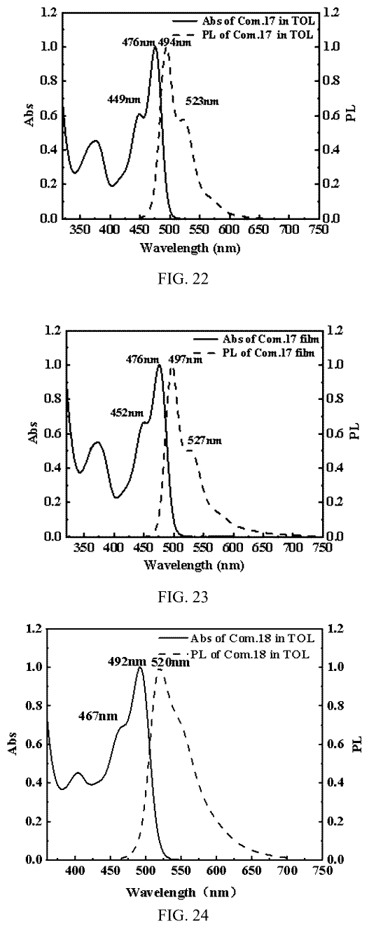

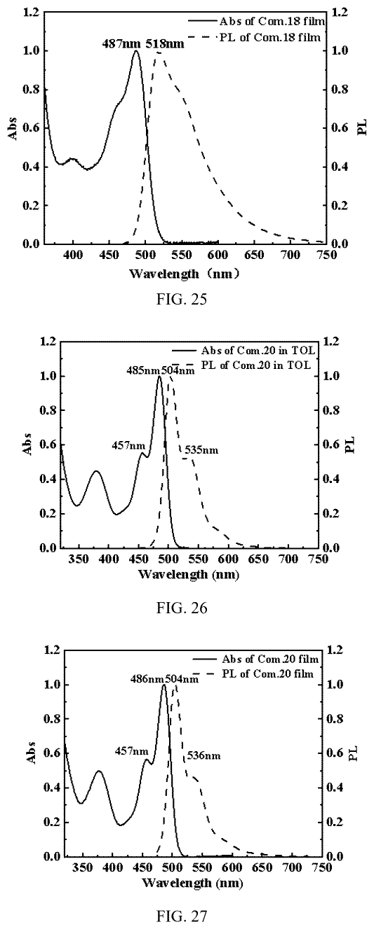

FIG. 2-FIG. 27 show the absorption and emission spectrums of the compounds 1, 3, 4, 5, 8, 9, 13, 14, 15, 16, 17, 18, 20 in the solution and film. It can be seen from these figures that the absorption and emission spectrums in the solution and film of the compound according to present disclosure are very similar, while the red shift of the spectrum in thin film is very small. This is due to the fact that in the aryl amines, the neighboring substitutions linked to the aryl group (here, benzene) effectively prevent molecular stacking in the film.

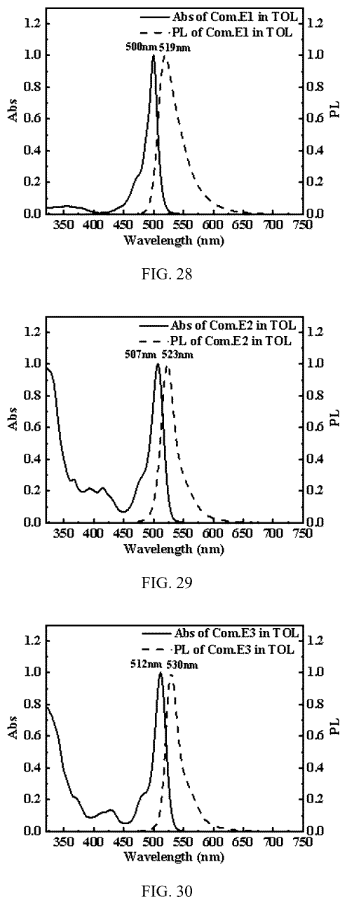

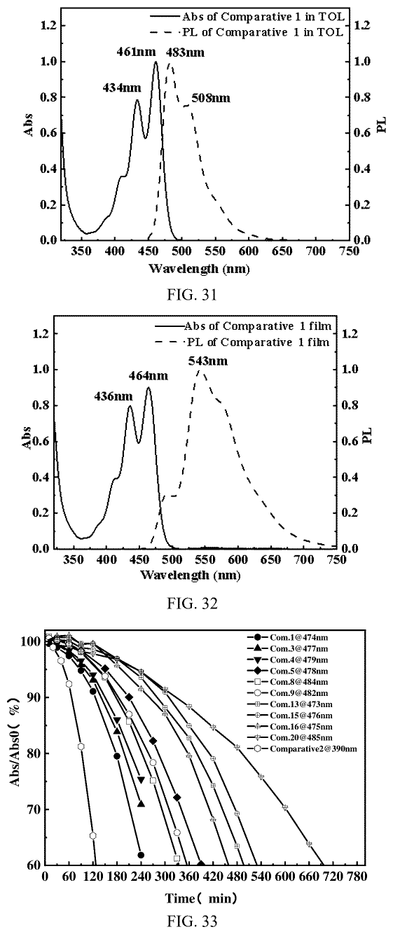

The optical properties of the compounds as described above (i.e., example 4): the absorption and emission spectrums were respectively measured by the spectrophotometer (Puxi T9s) and the fluorescent spectroscope (Hitachi, F-4700 FL Spectrophotometer). FIG. 28 shows the absorption and emission spectrums of the compound E1 in the toluene. FIG. 29 shows the absorption and emission spectrums of the compound E2 in the toluene. FIG. 30 shows the absorption and emission spectrums of the compound E3 in the toluene. FIG. 31 and FIG. 32 respectively show the absorption and emission spectrums of the comparative compound 1 in the toluene and film. The compound E1-compound E3 exhibit a narrow emission spectrum with a FWHM less than 40 nm. The emission spectrum of comparative compound 1 has a large red shift and the half-peak width became widened, which may affect the color purity of the green light, although it can still be used as a green or red host. In contrast, the film spectrums of the compounds as described herein are very similar to the solution spectrums with a very small red shift, which are more favorable for preparing the color conversion layers (CCLs) with high color purity.

### Example 5: UV Stability of Compounds

The UV stability of the compounds were tested as follows:

The compound was dissolved in the toluene with a concentration of 1×10⁻⁵ mol/L. 3 mL solution was added to the cuvette with a lid and the lid was screwed on tightly, then the cuvette was placed in the ultraviolet-visible spectrophotometer to test the absorption spectrum, the absorbance of the maximum absorption peak was recorded as the initial value. The cuvette was placed at a distance of 12 cm from the UV LEDs (365 nm & 255 nm), irradiated for a period and tested its absorption spectrum, then continued to be irradiated after the test, and repeated until the absorbance decayed to 80% of its initial value, the time was recorded as t80.

FIG. 33 shows the absorption decay of the compound 1, compound 3, compound 4, compound 5, compound 8, compound 9, compound 13, compound 15, compound 16, compound 20, and comparative compound 2 after UV irradiation in the toluene. The experimental results are tabulated below, where t80 is normalized by taking comparative compound 2 as 100%. The photostability of the compound as described herein is significantly improved compared with that of the comparative compound 2.

TABLE 4

| Materials | Times t80 | Materials | Times t80 |
|---|---|---|---|
| compound 1 | 190% | compound 13 | 410% |
| compound 3 | 212% | compound 15 | 440% |
| compound 4 | 234% | compound 16 | 383% |
| compound 5 | 304% | compound 20 | 528% |
| compound 8 | 261% | comparative | 100% |
| compound 9 | 279% | compound 2 | |

### Example 6: Blue Light Stability of the Film

The blue light stability of the films of the compound 8 and the comparative compound 1 were tested as follows:

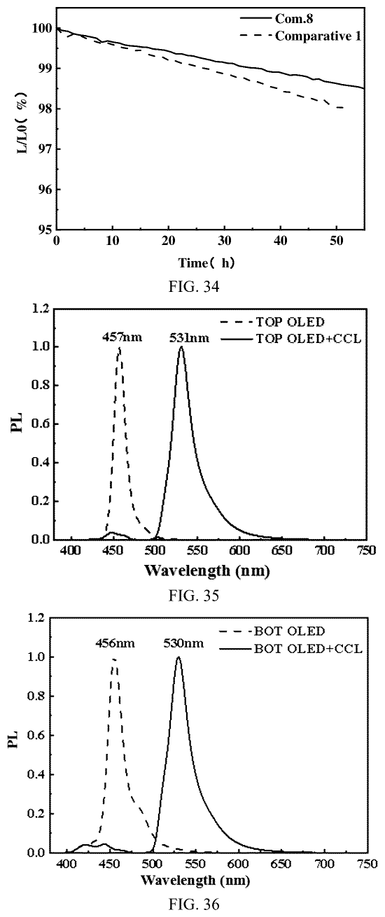

A film about 800 nm was evaporated, encapsulated with a glass cover plate, then placed at 2.5 cm above the blue LED (460 nm, 3000 cd/m²) to test the luminance value with a luminance meter (Fstar, CS-2000A). The luminance value of the first test was recorded as the initial value, and the luminance was tested by irradiating for a period of time to obtain the luminance decay curve, see FIG. 34. As seen in FIG. 34, the film of the compound 8 is obviously improved UV stability compared with that of the comparative compound 1.

### Example 7: Preparation of the Color Conversion Layer (CCL)

7.1 Evaporated films: compound 8 and light-emitting material E1, E2 or E3 were respectively placed in a crucible, and the crucibles were put into a thermal evaporation equipment. The cavity was vacuumed with the vacuum degree reaching 1×10⁻⁴ Pa, and then the crucibles began to be heated. The two organic compounds were deposited on the glass substrate by thermal evaporation. After the film reaching the target thickness, the crucibles were stopped heating, and then cooled down to 80° C. The cavity was filled with nitrogen to atmospheric pressure, and opened to obtain the evaporated CCL film.

### 7.2 Solution-processed Films:

48 mg of compound 8 was dissolved in 1 mL of toluene solution, stirred for 30 min, and then 2 mg of a light-emitting material E1, or E2, or E3 was dissolved in the above solution. After stirred for 30 min, the solution was dropped on a glass substrate, spun-coated and heated at 80° C. for 5 min in order to obtain a CCL film.

### 7.3 Resin Films:

48 mg of compound 8 was dissolved in 1 mL of resin solution, stirred for 30 min, and then 2 mg of a light-emitting material E1, or E2, or E3 was dissolved in the above solution. After stirred for 30 min, the solution was dropped on a glass substrate, spun-coated, then UV cured in order to obtain a CCL film.

CCL based on other organic compounds can be prepared in the same way according to 7.1, 7.2, or 7.3.

### Example 8: Results of OLEDs or LED+CCL Films

1. Blue top-emitting OLED+resin film of CCL: a resin film of CCL (E3 as an emitter) with the thickness of about 4 μm was prepared according the above-mentioned 7.3 on the light extraction surface of the blue top-emitting OLED.
2. Blue bottom-emitting OLED+resin film of CCL: a resin film of CCL (E3 as an emitter) with the thickness of about 4 μm was prepared according the above-mentioned 7.3 on the light extraction surface of the blue bottom-emitting OLED.
3. The spectrums of the blue top-emitting OLED, blue top-emitting OLED+resin film of the CCL, blue bottom-emitting OLED, and blue bottom-emitting OLED+resin film of the CCL were tested by the luminance meter (Fstar, CS-2000A). FIG. 35 shows the spectrums of the blue top-emitting OLED and that combined with the resin film of the CCL (E3 as an emitter). FIG. 36 shows the spectrums of the blue bottom-emitting OLED and that combined with the resin film of the CCL (E3 as an emitter). As shown in the figure below, the resin film of the CCL as described herein can basically absorb all the blue light of the OLED in top-emission and bottom-emission devices, and convert the blue light into narrow-spectrum green light.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. A compound, comprising a structural unit of formula (I), 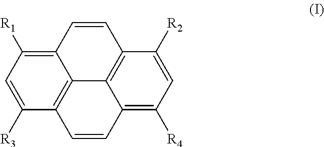
each of R₁ to R₄ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof;
wherein, at least three of R₁-R₄ are each independently selected from one of formulas (I-1)-(I-4): 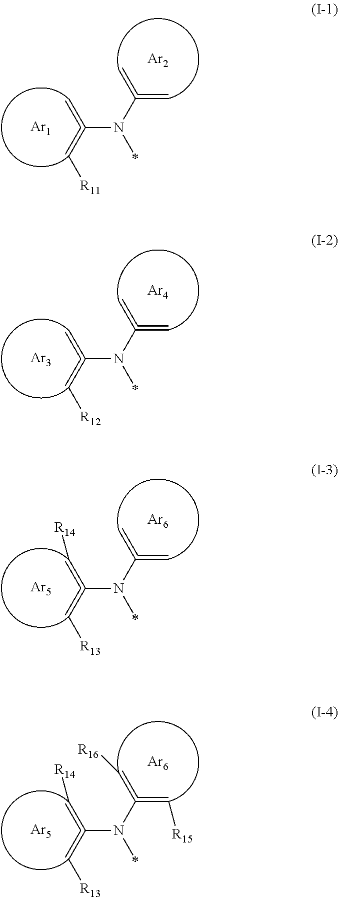
wherein:
each * independently represents an attachment site connecting a pyrene;
Ar₁ is a substituted/unsubstituted aromatic or heteroaromatic group containing 8 to 24 ring atoms;
each of Ar₂ to Ar₆ is a substituted/unsubstituted aromatic or heteroaromatic group containing 5 to 24 ring atoms, and formula (I-2) comprises an electron-withdrawing group;
each of R₁₁ to R₁₆ is a substituent and independently selected from a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, or any combination thereof.

2. The compound according to claim 1, wherein Ar₁ in formula (I-1) is selected from one of the following structural formulas or any combination thereof, which is further substituted: 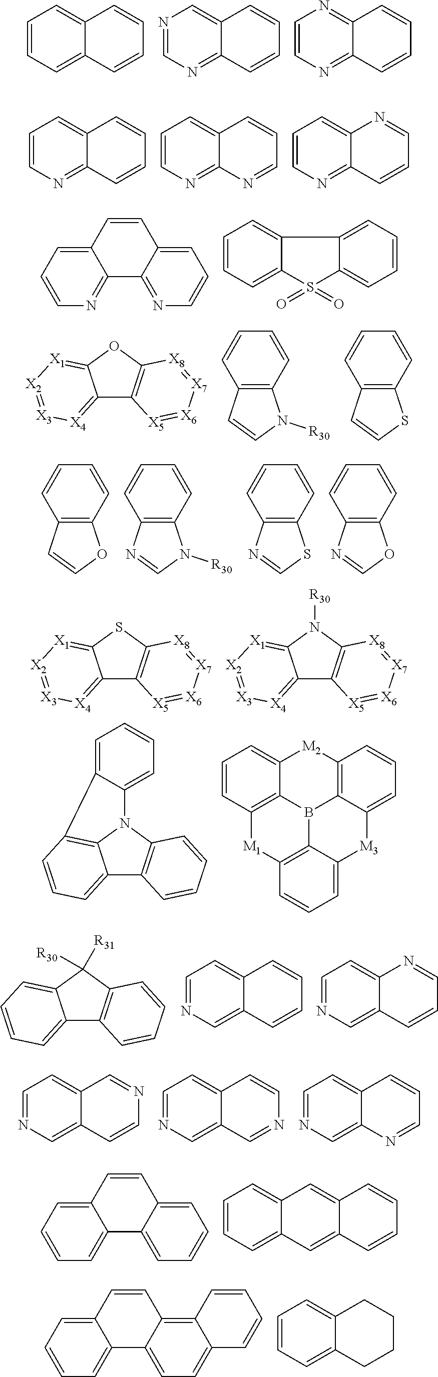 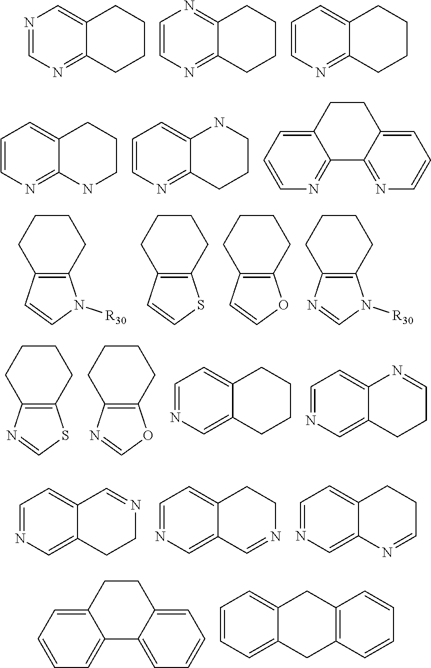
wherein: each of X₁ to X₈ is independently CR₃₂ or N; M₁, M₂, and M₃ are each independently selected from N(R₃₂), C(R₃₂R₃₃)₂, Si(R₃₂R₃₃)₂, O, C═N(R₃₂), C═C(R₃₂R₃₃)₂, P(R₃₂), P(═O)R₃₂, S, S═O, SO₂, or null; each of R₃₀ to R₃₃ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof, wherein one or more R₃₀-R₃₃ form a monocyclic or polycyclic aliphatic or aromatic ring system with each other and/or with the rings bonded thereto.

3. The compound according to claim 1, wherein each of Ar₂ to Ar₆ at each occurrence is independently selected from one of the following structural formulas or any combination thereof, which is further arbitrarily substituted:

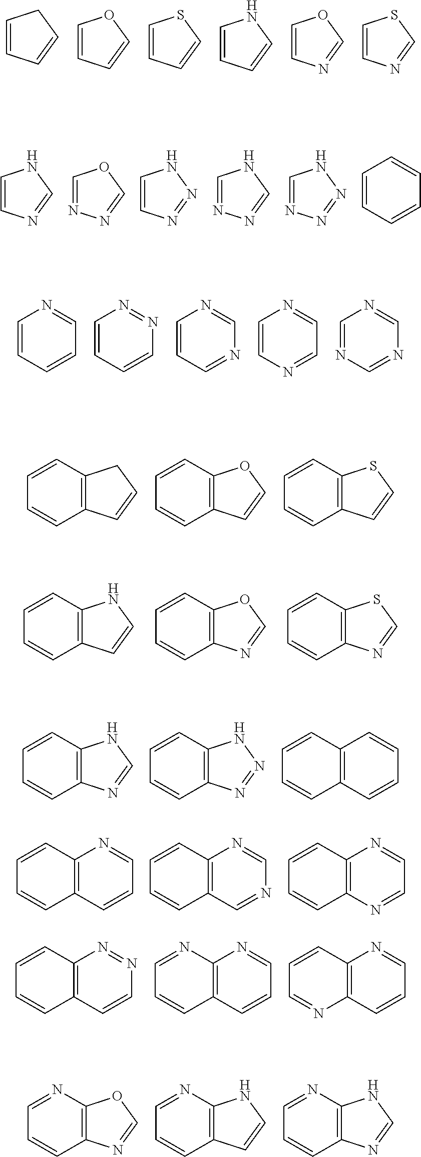

4. The compound according to claim 1, wherein the electron-withdrawing group in formula (I-2) is selected from F, a cyano group, a partial/perfluorinated alkyl chain, or one of the following groups: 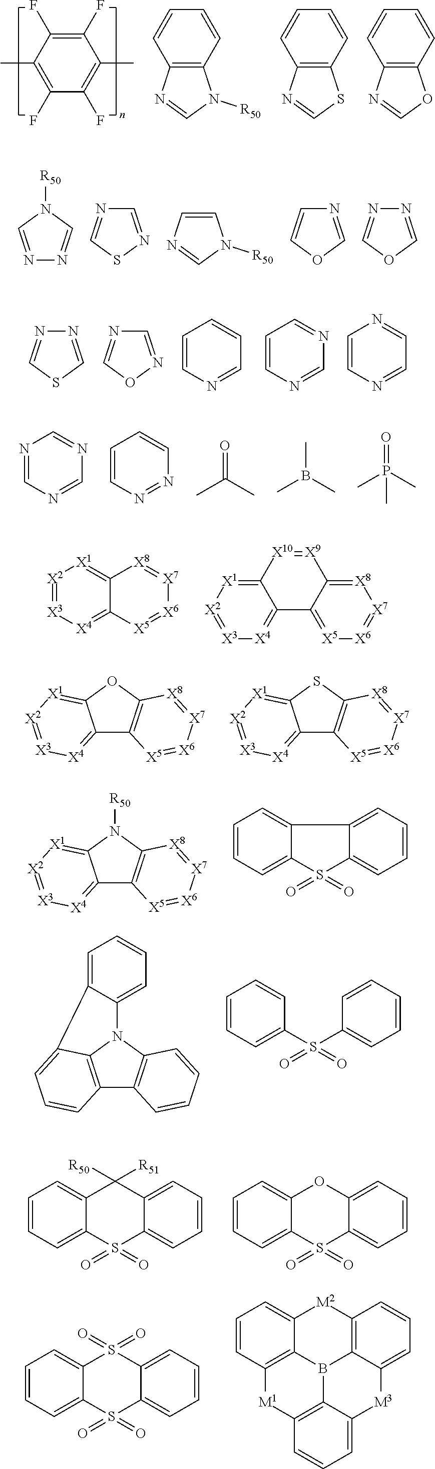
wherein: n is an integer from 1 to 3; each of X¹ to X¹⁰ is independently CR₅₂ or N, and at least one of them is N; M¹, M², and M³ are each independently selected from N(R₅₃), C(R₅₃R₅₄)₂, Si(R₅₃R₅₄)₂, O, C═N(R₅₃), C═C(R₅₃R₅₄)₂, P(R₅₃), P(═O)R₅₃, S, S═O, SO₂, or null; each of R₅₀ to R₅₄ at each occurrence is independently selected from —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₄-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, or any combination thereof, wherein one or more R₅₀-R₅₄ form a monocyclic or polycyclic aliphatic or aromatic ring system with each other and/or with the rings bonded thereto.

5. An organic functional film, comprising the compound according to claim 1.

6. An optoelectronic device, comprising the compound according to claim 1.

7. The optoelectronic device according to claim 6, wherein the optoelectronic device is an organic light-emitting device, comprising a substrate, a first electrode, an organic light-emitting layer, a second electrode, a color conversion layer, and an encapsulation layer in sequence from bottom to top, the second electrode is at least partially transparent, wherein
the color conversion layer comprises the compound, and an emitter E;
the color conversion layer at least partially absorbs the light emitted by the organic light-emitting layer through the second electrode;
the emission spectrum of the compound or the polymer is on the short wavelength side of the absorption spectrum of the emitter E, and at least partially overlaps with the absorption spectrum of the emitter E; and
the FWHM of the emission spectrum of the emitter E≤55 nm.

8. An optoelectronic device, comprising the organic functional film according to claim 5.

9. The optoelectronic device according to claim 8, wherein the optoelectronic device is an organic light-emitting device, comprising a substrate, a first electrode, an organic light-emitting layer, a second electrode, a color conversion layer, and an encapsulation layer in sequence from bottom to top, the second electrode is at least partially transparent, wherein
the color conversion layer comprises the compound, and an emitter E;
the color conversion layer at least partially absorbs the light emitted by the organic light-emitting layer through the second electrode;
the emission spectrum of the compound or the polymer is on the short wavelength side of the absorption spectrum of the emitter E, and at least partially overlaps with the absorption spectrum of the emitter E; and
the FWHM of the emission spectrum of the emitter E≤55 nm.
